(19) United States
(12) Patent Application Publication
(10) Pub. No.: US 2023/0394654 A1
(43) Pub. Date: Dec. 7, 2023

(54) METHOD AND SYSTEM FOR ASSESSING FUNCTIONALLY SIGNIFICANT VESSEL OBSTRUCTION BASED ON MACHINE LEARNING

(71) Applicant: Pie Medical Imaging B.V., Maastricht (NL)

(72) Inventors: Nils Hampe, Utrecht (NL); Ivana Isgum, Nieuwegein (NL); Sanne GM van Velzen, Castricum (NL); Jean-Paul Aben, Limbricht (NL)

(73) Assignee: Pie Medical Imaging B.V., Maastricht (NL)

(21) Appl. No.: 18/206,536

(22) Filed: Jun. 6, 2023

Related U.S. Application Data
(60) Provisional application No. 63/349,864, filed on Jun. 7, 2022.

(51) Int. Cl. G06T 7/00; G06T 7/11; A61B 6/00

(52) U.S. Cl. CPC G06T 7/0012; G06T 7/11; A61B 6/504; G06T 2207/30104; G06T 2207/30241; G06T 2207/20081; G06T 2207/20084; G06T 2207/30096; G06T 2207/30048; G06T 2207/30172; G06T 2207/10081

(57) ABSTRACT

Methods and systems are provided for assessing obstruction of a vessel of interest of a patient, which involve obtaining a volumetric image dataset for the vessel of interest. The volumetric image dataset is analyzed to extract data representing axial trajectory of the vessel of interest. A multi-planar reformatted (MPR) image is generated from the volumetric image dataset and the data representing axial trajectory of the vessel of interest; The MPR image is supplied as input to a first machine learning network that outputs feature data that characterizes a plurality of features of the vessel of interest along the axial trajectory of the vessel of interest given the MPR image. Additional data that characterizes at least one additional feature of the vessel of interest along the axial trajectory of the vessel of interest is generated by analysis separate and distinct from the first machine learning network. The data output by the first machine learning network and the additional data is input to a second machine learning network that outputs data that characterizes anatomical lesion severity of the vessel of interest given the input data.

### CROSS-REFERENCE TO RELATED APPLICATION(S)

The present application claims priority from U.S. Provisional App. No. 63/349,864, filed on Jun. 7, 2022, (Attorney Docket No. PIE-029P), herein incorporated by reference in its entirety.

### BACKGROUND

### 1. Field

The present application relates to the technical field of medical imaging, particularly computed tomography angiography imaging, although it can find application in any field where there is the need to quantify flow in obstructed or partially obstructed conduits such as in non-destructive testing applications.

### 2. State of the Art

Coronary artery disease (CAD) is one of the leading causes of death worldwide. CAD generally refers to conditions that involve narrowed or blocked blood vessels that can lead to reduced or absent blood supply to the sections distal to the stenosis resulting in reduced oxygen supply to the myocardium, resulting in, for instance, ischemia and chest pain (angina). Narrowing of a blood vessel is called stenosis and is caused by atherosclerosis which refers to the buildup of fats, cholesterol, and other substances in and on vessel walls (plaque), see FIG. 1. Atherosclerotic plaque can be classified according to its components, such as calcified plaque, soft plaque, and mixed plaque, i.e., plaque containing calcified and non-calcified components. Such non-calcified components include extracellular matrix, smooth muscle cells, macrophages, foam cells, lipid, and fibrous tissue. Calcified plaque is considered stable and its amount in the coronary artery is a strong predictor of cardiovascular events. Unlike calcified plaque, non-calcified plaque and mixed plaque are consider unstable and more prone to rupture. A plaque rupture may lead to acute major events such as a stroke, or a heart attack in case the rupture occurs in the coronary artery. A heart attack can result in a myocardium infarction resulting in irreversible damage to the myocardium. As different types of plaque and varying grades of stenosis lead to different patient management strategies, it is important to detect and characterize coronary artery plaque and stenosis grade.

Besides the grade of stenosis (anatomical stenosis), another important aspect in the prevention and treatment of CAD is the functional assessment of such narrowed anatomical stenosis or blocked blood vessels.

Presently, X-ray angiography is the imaging modality used during treatment of stenotic (narrowed) coronary arteries by means of a minimally invasive procedure also known as percutaneous coronary intervention (PCI) within the catheterization laboratory. During PCI, a (interventional) cardiologist feeds a deflated balloon or other device on a catheter from the inguinal femoral artery or radial artery up through blood vessels until they reach the site of blockage in the artery. X-ray imaging is used to guide the catheter threading. PCI usually involves inflating a balloon to open the artery with the aim of restoring unimpeded blood flow. Stents or scaffolds may be placed at the site of the blockage to hold the artery open. For intermediate coronary anatomical lesions (defined as luminal narrowing of 30-70%), for instance, it is not always obvious if stenosis is a risk for the patient and if it is desired to take action. Overestimation of the severity of the stenosis can cause a treatment which in hindsight would not have been necessary and therefore expose the patient to risks that are not necessary. Underestimation of the severity of the stenosis, however, could induce risks because the patient is left untreated while the stenosis is in reality severe and actually impedes flow to the myocardium. Especially for these situations it is desired to have an additional functional assessment to aid in good decision making.

Fractional Flow Reserve (FFR) has been used increasingly over the last 10-15 years as a method to identify and effectively target the coronary lesion most likely to benefit from PCI. FFR characterizes pressure differences across a coronary artery stenosis to determine the likelihood that the stenosis impedes oxygen delivery to the heart muscle. The characterization of FFR typically involves percutaneously inserting a pressure-transducing wire inside the coronary artery and measuring the pressure behind (distal to) and before (proximal to) the lesion and is performed in the catheterization laboratory. This is best done in a hyperemic state because in the case of maximum hyperemia, blood flow to the myocardium is proportional to the myocardium perfusion pressure. FFR therefore provides a quantitative assessment of the functional severity of the coronary lesion as described in Pijls et al. in “Measurement of Fractional Flow Reserve to Assess the Functional Severity of Coronary-Artery Stenoses”, N Engl J Med 1996, 334:1703-1708. Although the European Society of Cardiology (ESC) and the American College of Cardiology/American Heart Association (ACC/AHA) guidelines recommend the use of FFR in patients with intermediate coronary stenosis (30-70%), visual assessment, whether or not supported by QCA, of X-ray coronary angiograms alone is still used in over 90% of procedures to select patients for percutaneous coronary intervention (Kleiman et al, “Bringing it all together: integration of physiology with anatomy during cardiac catheterization”, J Am Coll Cardiol. 2011; 58:1219-1221). FFR, however, has some disadvantages. For example, characterizing FFR can be associated with the additional cost of a pressure wire which can only be used once. Furthermore, characterizing FFR can require invasive catheterization with the associated cost and procedure time. Also, in order to induce (maximum) hyperemia, additional drug infusion (adenosine or papaverine) can be required, which is an extra burden for the patient.

Coronary computed tomography (CT) angiography (CCTA) is a non-invasive imaging modality for the anatomic assessment of coronary arteries but does not assess the functional significance of coronary lesions. Due to the remarkably high negative predictive value of CCTA and its non-invasive nature, the main strength of CCTA is its excellent ability to exclude CAD. Although CCTA can reliably exclude the presence of significant coronary artery disease, many high-grade stenosis seen on CCTA are not flow limiting. This potential for false positive results has raised concerns that widespread use of CCTA may lead to clinically unnecessary coronary revascularization procedures. This lack of specificity of CCTA is one of the main limitations of CCTA in determining the hemodynamic significance of CAD (Meijboom et al, “Comprehensive assessment of coronary artery stenoses: computed tomography coronary angiography versus conventional coronary angiography and correlation with fractional ow reserve in patients with stable angina”, Journal of the American College of Cardiology 52 (8) (2008) 636-643). As a result, CCTA may lead to unnecessary interventions on the patient, which may pose added risks to patients and may result in unnecessary health care costs.

In Taylor et al “Computational Fluid Dynamics Applied to Cardiac Computed Tomography for Noninvasive Quantification of Fractional Flow Reserve”, Journal of the American College of Cardiology, Vol. 61, No. 22, 2013, and U.S. Pat. No. 8,315,812, a noninvasive method for quantifying FFR from CCTA is described (FFRCT). This technology uses computational fluid dynamics (CFD) applied to CCTA after semi-automated segmentation of the coronary tree including a part of the ascending aorta covering the region in which both the left coronary artery as well as the right coronary artery emanate. Three-dimensional (3D) blood flow and pressure of the coronary arteries are simulated, with blood modeled as an incompressible Newtonian fluid with Navier-Stokes equations and solved subject to appropriate initial and boundary conditions with a finite element method on parallel supercomputer. The FFRCT is modeled for conditions of adenosine-induced hyperemia without adenosine infusion. This process is computationally complex and time-consuming and may require several hours and heavily relies on the 3D anatomical coronary model as a result of the segmentation which suffers amongst others from the same limitation as described above.

In addition to development of CFD-based FFR prediction methods, approaches emerged that correlate quantitative indices derived from CCTA with measured FFR value. These clinical indices characterize a coronary artery through e.g., transluminal attenuation gradient (Wong et al., 2013; Ko et al., 2016) or plaque volume (Diaz-Zamudio et al., 2015; Otaki et al., 2021), or describe specific lesions by quantifying degree of stenosis (Gould et al., 1975; Otaki et al., 2021) or contrast density difference (Dey et al., 2014; Hell et al., 2015). While the mathematical simplicity and intuitive design of the calculated indices enables their interpretation, it limits their capability to model the complex relationship between FFR and the coronary artery characteristics on CCTA. Hence, to improve FFR prediction with clinical indices, machine learning classifiers were employed that combined multiple indices (Ko et al., 2015; Itu et al., 2016; Dey et al., 2018; Otaki et al., 2021; Yang et al., 2021). This led to a substantial performance increase compared to the performance of a single index. However, these index-based works share a drawback with CFD-based methods: calculating the indices requires accurate segmentation of the coronary artery lumen, which can be highly challenging, especially in the presence of pathology Ghanem et al. (2019). While these methods typically use an automatic segmentation method as a starting point, errors in the automatic segmentation regularly necessitate substantial manual interaction.

There is thus the need for obtaining coronary artery lesion parameters (such as plaque type, anatomical lesion severity and functional coronary lesion severity) without relying on the detailed morphology of the coronary arterial system.

### SUMMARY

In accordance with aspects herein, a method is provided for assessing a vessel obstruction through deep learning-based analysis of volumetric image data.

In earlier deep learning work (U.S. application Ser. Nos. 15/933,854, and 16/379,248), several methods were disclosed to assess functional severity of vessel obstruction(s) by focusing on a region of interest extracting the myocardium and/or an MPR for the artery of interest.

In the present application, new deep learning methods and systems are provided that use a convolutional neural network (CNN) or variational autoencoder to extract additional features or characteristics along a vessel of interest. Additionally, features or characteristics can be extracted directly from a coronary artery centerline tree, where such features indicate per coronary artery centerline point whether it is in a main artery or side-branch and whether a bifurcation is present at that location. These features can be used in combination with other extracted features to assess vessel obstruction. For this purpose, a second network is trained to perform both regression of the FFR value, FFR drops, pullback FFR and classification of the functional significance of an artery obstruction.

In embodiments herein, a method for assessing obstruction of a vessel of interest of a patient, comprises:
obtaining a volumetric image dataset, for example CCTA image data, for the vessel of interest, such as, for example, a coronary artery or a coronary tree;
analyzing the volumetric image dataset to extract data representing axial trajectory of the vessel of interest;
generating a multi-planar reformatted (MPR) image based on the volumetric image dataset and the data representing axial trajectory of the vessel of interest;
supplying the MPR image as input to a first machine learning network that outputs feature data that characterizes a plurality of features of the vessel of interest along the axial trajectory of the vessel of interest given the MPR image;
generating additional data that characterizes at least one additional feature of the vessel of interest along the axial trajectory of the vessel of interest by analysis separate and distinct from the first machine learning network; and
supplying the data output by the first machine learning network and the additional data as input data to a second machine learning network that outputs data that characterizes anatomical lesion severity of the vessel of interest given the input data.

The method may further comprise displaying or outputting the data that characterizes anatomical lesion severity of the vessel of interest.

The additional data may be generated from analysis of the MPR image and/or from analysis of the volumetric image dataset and/or from a coronary artery centerline tree derived from the volumetric image dataset.

The additional data may characterize at least one of side branches and bifurcations along the axial trajectory of the vessel of interest and/or at least one of soft plaque area, mixed plaque area, or other characteristic feature along the axial trajectory of the vessel of interest.

The additional data may further characterize a localized part of the myocardium that is associated with the vessel of interest.

In an improvement, the data output by the second machine learning network includes a fractional flow reserve (FFR) value for the entire vessel of interest and the second machine learning network may be advantageously trained by supervised learning using training data that includes reference annotations based on measurements of FFR values for a plurality of patients.

In a further improvement, the data output by the second machine learning network includes fractional flow reserve (FFR) values for centerline points along the vessel of interest and the second machine learning network is trained by supervised learning using training data that includes reference annotations based on measurements of FFR values associated with vessel centerline points for a plurality of patients.

In a still further improvement, the data output by the second machine learning network represents a prediction for the presence of a functionally significant stenosis and the second machine learning network is trained by supervised learning using training data that includes reference annotations representing presence of a functionally significant stenosis for a plurality of patients.

In an embodiment, the plurality of the features characterized by the feature data output by the first machine learning network includes at least one feature related to lumen characteristics of the vessel of interest (such as lumen area and/or lumen attenuation) along the axial trajectory of the vessel of interest.

In an improvement, the plurality of the features characterized by the feature data output by the first machine learning network includes at least one feature related to plaque characteristics of the vessel of interest (such as calcium plaque area, soft plaque area, mixed plaque area) along the axial trajectory of the vessel of interest.

The first machine learning network may advantageously comprise a convolutional neural network, which is trained using training data that includes reference annotations for the plurality of the features characterized by the feature data output by the first machine learning network.

The reference annotations may be derived by manual segmentation of corresponding volumetric image data and/or automatic segmentation of corresponding volumetric image data.

The second machine learning network may advantageously comprise a convolutional neural network, which is trained using training data that includes volumetric image data and corresponding reference annotations for the output data that characterizes anatomical lesion severity of the vessel of interest.

The reference annotations may be derived by manual segmentation of the corresponding volumetric image data and/or automatic segmentation of the corresponding volumetric image data.

The convolutional neural network of the second machine learning system may include a regression head that outputs a fractional flow reserve (FFR) value.

In an improvement, the convolutional neural network of the second machine learning system further includes an accumulator that outputs fractional flow reserve (FFR) values for centerline points along the vessel of interest.

The convolutional neural network of the second machine learning system may include a classification head that outputs data representing a prediction for the presence of a functionally significant stenosis.

According to an aspect, embodiments herein also relate to a system for assessing obstruction of a vessel of interest of a patient, the system comprising at least one processor that, when executing program instructions stored in memory, is configured to perform some or all operations of the method according to embodiments herein.

The system may advantageously comprise an imaging acquisition subsystem configured to acquire the volumetric image dataset and/or a display subsystem configured to display the data that characterizes anatomical lesion severity of the vessel of interest.

Further variants are possible. For example, an embodiment involves generating first feature data that characterizes presence of zero or more bifurcations or side branches along the axial trajectory of the vessel of interest to be supplied to the first machine learning network.

In another embodiment, some or all of the additional feature data and/or the MRP image are adjusted based on simulated or planned treatment of the vessel of interest.

In a further embodiment, the first machine learning network may be advantageously configured to output a plurality of latent space encodings that characterizes features of the vessel of interest along the axial trajectory of the vessel of interest given the MPR image and/or additional feature data that characterizes additional features of the vessel of interest along the axial trajectory of the vessel of interest. The plurality of latent space encodings and/or the additional feature data output by the first machine learning network may be supplied to the second machine learning network that outputs data that characterizes FFR pullback of the vessel of interest which accounts for the simulated or planned treatment of the vessel of interest given the input data.

Embodiments may also provide methods and systems for extracting a coronary tree from volumetric image data of a vessel of interest of a patient, which include one, some or all of the following operations:
obtaining a volumetric image dataset for the vessel of interest;
tracking a plurality of seed points in the image dataset;
using the plurality of seed points to extract an initial representation of a coronary tree in the image dataset;
inputting the initial representation of the coronary tree to a first ensemble of graph convolutional neural networks to generate a refined representation of the coronary tree;
using a second ensemble of graph convolutional neural networks to generate labels for segments of the refined representation of the coronary tree.

Other aspects are described and claimed hereinafter.

### BRIEF DESCRIPTION OF THE DRAWINGS

The characteristics of the present disclosure and the advantages derived therefrom will be more apparent from the following description of non-limiting embodiments, illustrated in the annexed drawings described below.

FIG. 1 shows an example of coronary atherosclerosis.

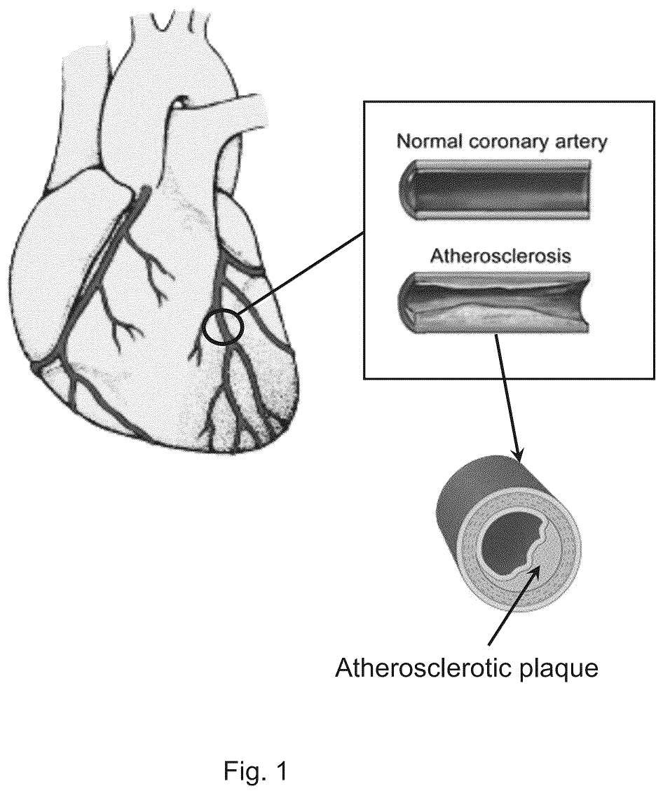

FIG. 2 illustrates a flowchart of a machine learning based method for determining functionally significant lesion severity in one or more coronary arteries to an embodiment of the present application.

FIG. 3 shows a functional block diagram of an exemplary CT system.

FIG. 4 illustrates a coronary centerline tree extracted from a CCTA dataset.

FIGS. 5a-5e illustrate the creation of a volumetric MPR image.

FIG. 6 shows an example of the architecture of the network for extracting artery characteristics.

FIGS. 7a-7c show a schematic illustration of a co-registration method of pullback FFR reference values with the CCTA image dataset.

FIG. 8 shows an example of the architecture of the network for extracting five artery characteristics.

FIG. 9 is a schematic illustration of the stenosis assessment network.

FIG. 10 is a schematic illustration of an extension/adjustment to the stenosis assessment network (FIGS. 7a-7c) to include an FFR value per centerline point along a vessel of interest as additional output.

FIG. 11 illustrates an exemplary graphical user interface (display screens) that displays FFR values for centerline points of a vessel of interest.

FIG. 12 shows an example of obtaining a computed FFR pullback of the coronary circumflex by using a CAAS Workstation.

FIG. 13 is a schematic illustration of an extension/adjustment to the stenosis assessment network (FIG. 9 or FIG. 10) to include the extraction of soft plaque area and mixed plaque area as described by Isgum et al. in U.S. Pat. No. 10,699,407B2 for use in the stenosis assessment network.

FIG. 14 is a schematic illustration of an extension/adjustment to the deep learning networks (FIG. 9, FIG. 10, FIG. 13) to include the extraction of data characterizing the myocardium of the heart for use in the ‘machine learning based stenosis assessment’ network.

FIG. 15 is a schematic illustration of a heart with the myocardium of the heart subdivided into regions covered by the coronary arteries.

FIG. 16 is another schematic illustration of an extension/adjustment to the deep learning networks (FIG. 9, FIG. 10, FIG. 13) to include the extraction of data characterizing the myocardium of the heart for use in the ‘machine learning based stenosis assessment’ network.

FIG. 17 illustrates a flowchart of a machine learning based method for determining functionally significant lesion severity along the axial trajectory of a vessel of interest (e.g., one or more coronary arteries) according to an embodiment of the present application.

FIG. 18 shows a high-level schematic illustration of the machine learning based method as described by the flowchart of FIG. 17.

FIG. 19a is a schematic illustration of the architecture of the deployed variational autoencoder.

FIG. 19b is a schematic illustration of the architecture of the deployed variational autoencoder illustrates the unsupervised and supervised part of the variational autoencoder.

FIG. 20 shows an example of a network architecture for the FFR pullback network.

FIG. 21 a schematic illustration of a combined network of the machine learning based stenosis assessment network (206, FIG. 2) with the machine learning based FFR pullback network (1706, FIG. 17).

FIG. 22 provides an illustration for integrating myocardium characteristics with respect to the machine learning based FFR pullback network architecture from FIG. 21.

FIG. 23 illustrates a flowchart of a machine learning based method for simulating virtual stent placement according to an embodiment of the present application.

FIG. 24 provides an illustration of altering the artery characteristics for simulation of successful PCI treatment.

FIG. 25 illustrates a method to automatically define the lesion segment and compute the healthy reference area within the lesion segment.

FIG. 26 illustrates an automatic method to simulate virtual stent placement utilizing the dense latent space of the variational autoencoder.

FIG. 27 illustrates incorrect virtual stent placement with the method as described with reference to FIG. 26.

FIG. 28 illustrates a flowchart of a machine learning based method for automatically extraction and anatomical labeling of the coronary tree according to an embodiment of the present application.

FIG. 29 illustrates a high-level methodology of the flowchart described by FIG. 28.

FIG. 30 illustrates merging of sub-graphs in the case of overlapping.

FIG. 31 illustrates the extraction of the coronary tree at multiple steps of the graph tracking.

FIG. 32 illustrates leakage of the tracking of a coronary artery centerline into a coronary vein, and by dividing the segment into smaller sub-segments yields well defined segments for tree refinement. This allows retaining the centerline in the coronary artery and removing the centerline in the coronary vein.

FIG. 33 illustrates the network architecture used for the GCN's related to the flowchart of FIG. 28.

FIG. 34 provides an illustration of the result of the flowchart of FIG. 29, which shows the extracted coronary tree in which each coronary segment is anatomically labelled.

FIGS. 35a-35d provide illustrations of coronary tree extraction when initiated from a seed within coronary artery and from a seed within coronary vein.

FIG. 36 shows a high-level block diagram of an example of a CT system.

FIG. 37 shows an example computer system.

### DETAILED DESCRIPTION OF EMBODIMENTS

The term “unseen”, as used throughout, refers to items which has not been used during the training phase. Item in this context means, a volumetric image, a reference value, features and/or other things used during the training phase to train the machine learning model. Instead, the unseen features, images, geometries, and other unseen items refer to aspects of a patient or object of interest that is being analyzed during the prediction phase of operation.

The term “FFR value” refers to an FFR value at a certain position within a vessel. In case FFR value is used without reference to position (centerline position, it can refer to the FFR value at the most distal position within the vessel of interest.

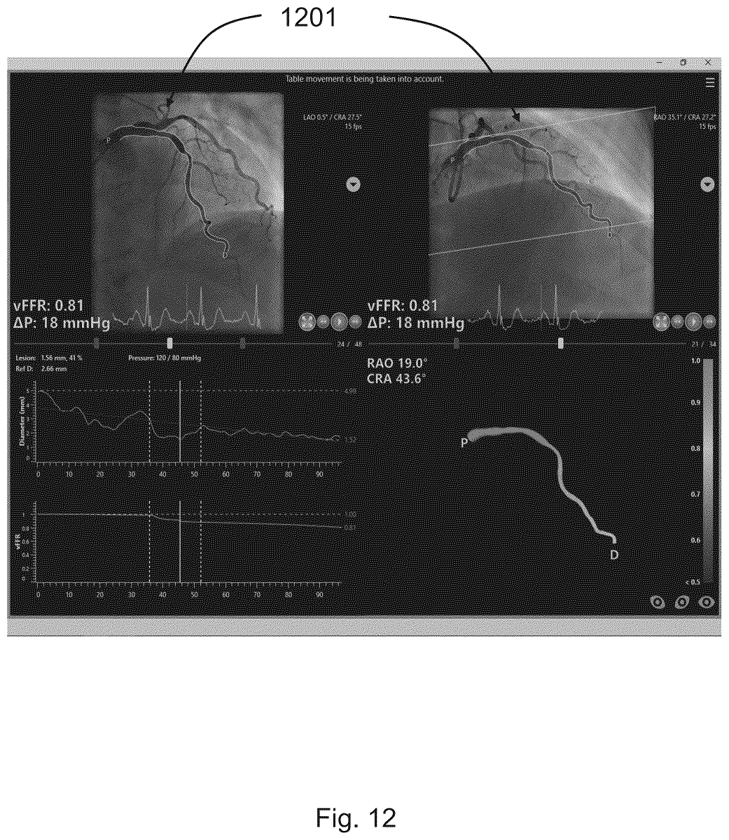

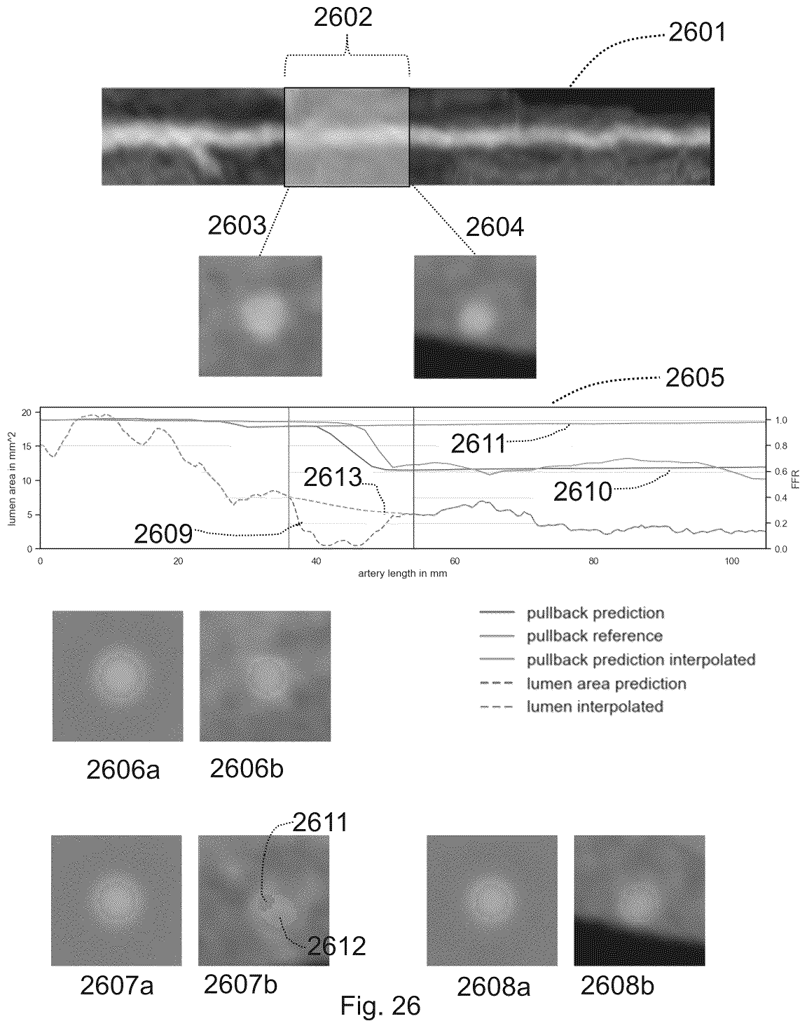

The term “FFR pullback” or “FFR pullback graph” refers to FFR values along the axial trajectory of a vessel of interest, as for instance illustrated by 1203 of FIG. 12 or 2610 of FIG. 26.

The term “FFR drop” means the decay in FFR values along the axial trajectory of a vessel of interest from the proximal end to the distal end of the vessel. The steepness of such decay allows separation between focal coronary artery disease and diffuse coronary artery disease. Focal coronary artery disease can be defined as an abrupt pressure drop (FFR drop) in FFR pullback within a relatively short vessel segment. On the other hand, diffuse coronary artery disease is defined as a gradual pressure loss (FFR drop) along the axial trajectory of the vessel without significant abrupt pressure drop at any position along the vessel.

Focal lesions are local obstructions that can be treated by dilating the stenosis using balloon that can be inflated possibly followed by placing a stent or scaffold. Diffuse lesions require different treatment approaches and need to be distinguished from focal lesions to prevent unnecessary costs, patient risks, and patient comfort by non-optimal treatment decisions. Therefore, the FFR pullback can be used to determine if a vessel has a focal or diffuse lesion based on the shape of the virtual pullback.

Throughout the present specification, terms which are common in the field of machine learning/deep learning are used. For detailed explanation of these terms a reference is made to Litjens et al, “A survey on deep learning in medical image analysis”, Med Image Anal. 2017 December; 42:60-88, Suganyadevi et al, “A review on deep learning in medical image analysis”, International journal of multimedia information retrieval vol. 11,1 (2022), Varoquaux et al, “Machine learning for medical imaging: methodological failures and recommendations for the future”, NPJ digital medicine vol. 5,1 48. 12 Apr. 2022; herein incorporated by reference in their entireties.

The present application relates to methods and systems for machine learning to assess functionally significant vessel obstruction of one or more vessel(s) of a target organ based on contrast enhanced volumetric image dataset. Machine learning is a subfield of computer science that “gives computers the ability to learn without being explicitly programmed”. Evolved from the study of pattern recognition and computational learning theory in artificial intelligence, machine-learning explores the study and construction of algorithms that can learn from and make predictions on data—such algorithms overcome following strictly static program instructions by making data driven predictions or decisions, through building a model from sample inputs. Machine-learning is employed in a range of computing tasks where designing and programming explicit algorithms is infeasible. Given a dataset of images with known class labels, machine-learning system can predict the class labels of new images, furthermore, machine-learning can also be unsupervised which uses machine learning algorithms to analyze and cluster unlabeled datasets. These algorithms discover hidden patterns or data groupings without the need for human intervention. On high level, machine learning can be split into two phases; 1) a training phase, in which the model is trained to learn specific features of a task (for instance FFR prediction), and 2) a testing/validation phase in which the trained model is deployed on unseen data to perform the task (for instance prediction of FFR).

In embodiments, the target organ can be coronary arteries or vessels and possibly the heart or portions thereof. A functionally significant vessel obstruction (also called stenosis or lesion) is a hemodynamically significant obstruction of a vessel, and with respect to coronary arteries it defines the likelihood that coronary artery obstruction(s) impedes oxygen delivery to the heart muscle and causes anginal symptoms. Fractional flow reserve is a hemodynamic index for assessment of functionally significant coronary artery obstruction(s). In addition to fractional flow reserve, other hemodynamic indices can be used to assess functionally significant coronary artery obstruction(s), such as coronary flow reserve, instantaneous wave-free ratio, hyperemic myocardium perfusion, index of microcirculatory resistance and pressure drop along a coronary artery.

Embodiments of the present application utilize machine learning to determine coronary parameters related to CAD such as functional severity of one or more vessel obstructions from a CCTA dataset. Machine learning is a subfield of computer science that “gives computers the ability to learn without being explicitly programmed”. Evolved from the study of pattern recognition and computational learning theory in artificial intelligence, machine-learning explores the study and construction of algorithms that can learn from and make predictions on data—such algorithms overcome following strictly static program instructions by making data driven predictions or decisions, through building a model from sample inputs. Machine-learning is employed in a range of computing tasks where designing and programming explicit algorithms is infeasible.

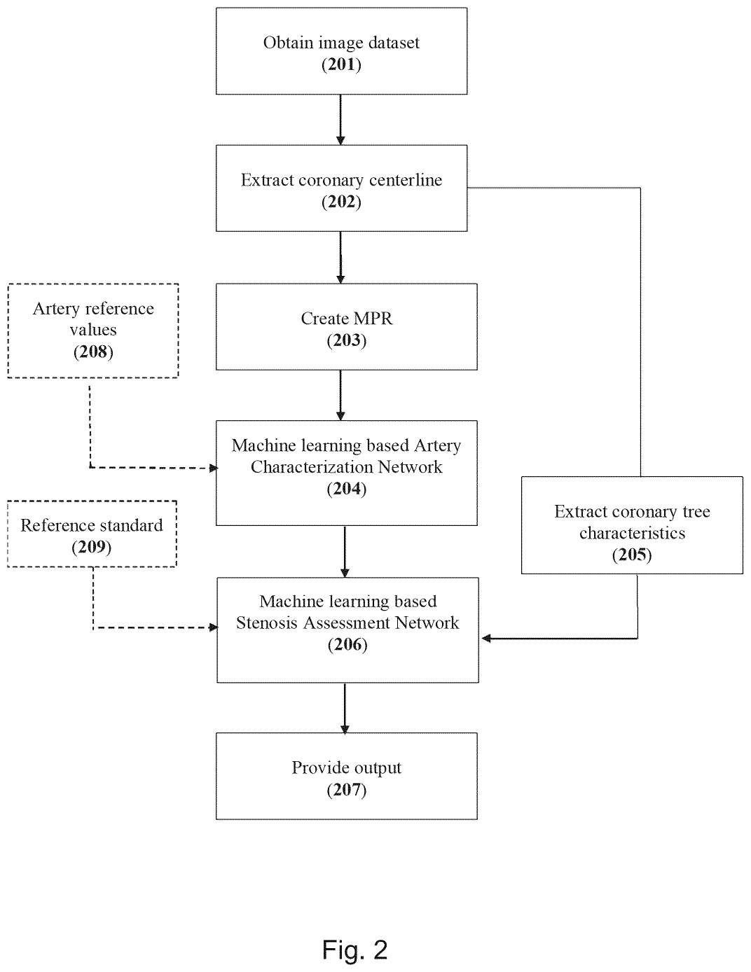

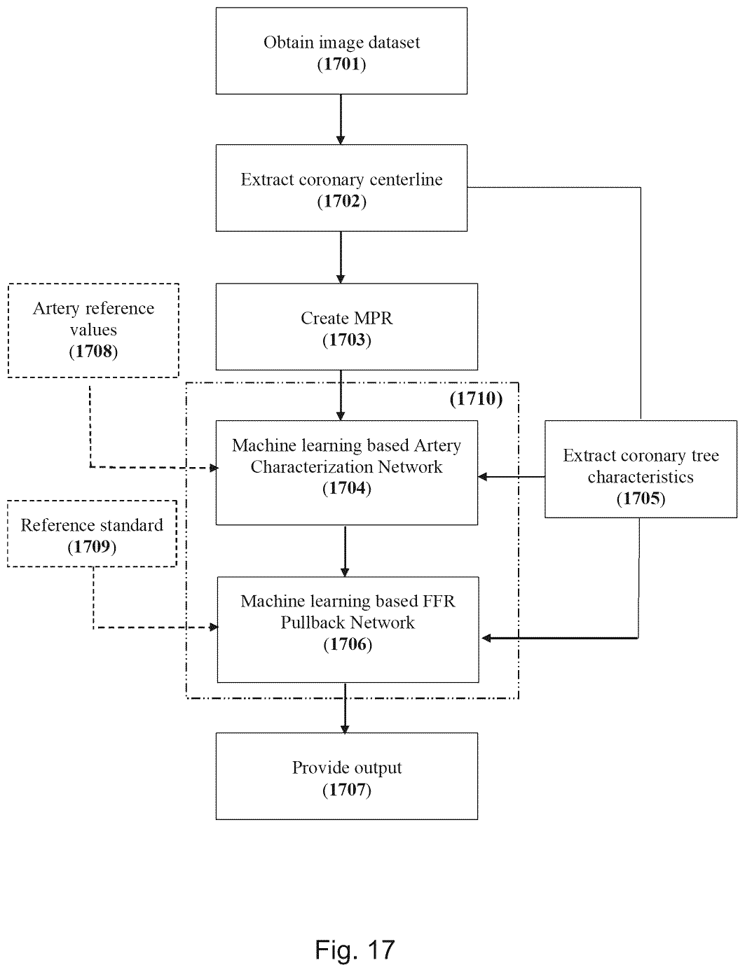

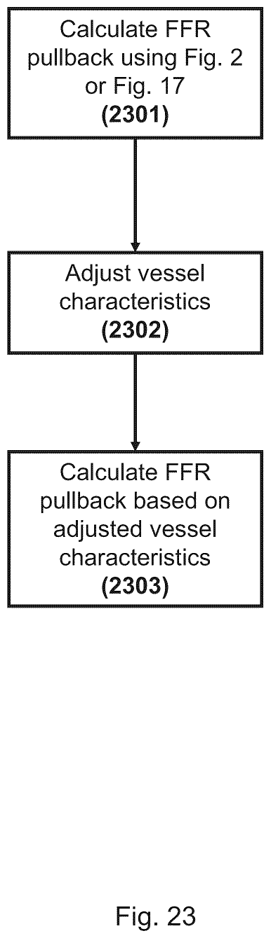

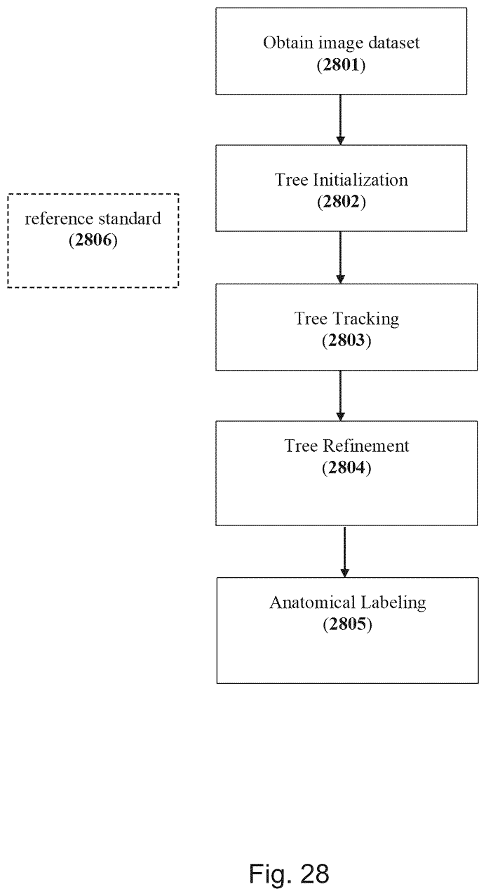

FIG. 2, FIG. 17, FIG. 23, and FIG. 28 each depict a flow chart illustrating operations according to an embodiment of the present application. The operations employ an imaging system capable of acquiring and processing a CCTA dataset of an organ (or portion thereof) or other object of interest. The operations of FIG. 2, FIG. 17, FIG. 23, and FIG. 28 (as well as the operations of any other methods, algorithms and processes described herein) are implemented by one or more processors, while executing program instructions. The one or more processors may be implemented on various computing devices, such as a smart phone, tablet device, laptop computer, desktop computer, workstation, remote server, cloud sever, medical network and the like. Alternatively, the one or more processors may be distributed between one or more separate computing devices, such that a portion of the operations are performed by one computing device, while the remainder of the operations are performed by one or more other computing devices.

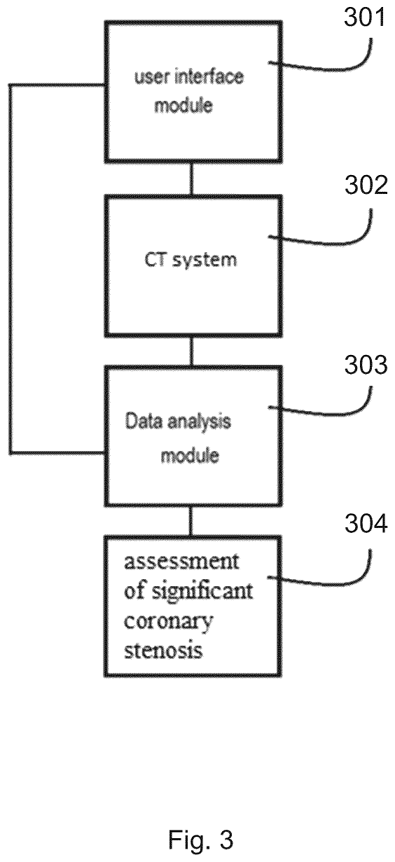

FIG. 3 is a functional block diagram of an exemplary CT system, which includes a CT imaging apparatus 302 that operates under commands from a user interface module 301 and will provide data to the data analysis module 303. A clinician or other user acquires at least one CT image of a patient to obtain CCTA image data of a volume of interest, for example the coronary arteries surrounding the heart of the patient. The CCTA image data can be stored in DICOM format on a hard disk, a PACS server, a network server, or a cloud server. The data analysis module 303 may be realized by a personal computer, workstation, or other computer processing system. The data analysis module 303 processes the acquired CCTA image data of the CT system 302 to generate, for instance, coronary analysis quantification.

The user interface module 301 interacts with the user and communicates with the data analysis module 303. The user interface module 301 can include different kinds of input and output devices, such as a display screen for visual output, a touch screen for touch input, a mouse pointer or other pointing device for input, a microphone for speech input, a speaker for audio output, a keyboard and/or keypad for input, etc. Module 304 provides for assessment of significant coronary stenosis of the patient. To assess the functional significance of stenosis in a vessel of interest of the patient, module 304 is configured to apply deep learning as described in present application directly to the raw CCTA image data acquired by the system. To enable robust training with limited data, this task can be divided using two subsequent networks, an artery characterization network (FIG. 2, 204, or FIG. 17, 1704) and a stenosis assessment network (FIG. 2, 206), or FFR pullback network (FIG. 17, 1706). Pre-processing for the artery characterization network (FIG. 2, 202, or FIG. 17, 1702) extracts data representing the axial trajectory of the vessel of interest from CCTA image data of the vessel of interest and generates a multiplanar reconstruction (MPR) of the vessel of interest from the extracted axial trajectory and the image data (FIG. 2, 203, of FIG. 17, 1703). The MPR can be represented by a volumetric (three-dimensional or “3D”) MPR image of the vessel of interest and/or a two-dimensional (or “2D”) MPR image of the vessel of interest.

The operations of FIG. 2, FIG. 17, FIG. 23, or FIG. 28 can be carried out by software code that is embodied in a computer product (for example, an optical disc or other form of persistent memory such as a USB drive, a network server, or a cloud server). The software code can be directly loadable into the memory of a data processing system for carrying out the operations of FIG. 2, FIG. 17, FIG. 23, or FIG. 28.

In this example it is assumed that the imaging system has acquired and stored at least one CCTA dataset of the vessel of interest. Any imaging device capable of providing a CT scan can be used for this purpose.

The present application is particularly advantageous in coronary artery lesion parameters analysis based on CCTA dataset and it will mainly be disclosed with reference to this field, particularly for patient classification.

An embodiment of the present application is now disclosed with reference to FIG. 2. The therein-depicted steps can, obviously, be performed in any logical sequence and can be omitted in parts.

In step 201 of FIG. 2, a CCTA image dataset is obtained. Such CCTA image dataset can represent a volumetric CCTA image dataset, such as a single contrast enhanced CCTA dataset. This CCTA dataset can be obtained from a digital storage database, such as an image archiving and communication system (PACS) or a VNA (vendor neutral archive), a local digital storage database, a cloud database, or acquired directly from a CT imaging modality. The CCTA dataset can be acquired by CT imaging operations where a contrast agent is injected to enhance the vessel of interest (e.g., coronary arteries or vessels). At least one aim of CCTA is to identify cardiac and coronary artery anatomy by means of injecting an exogenous contrast agent, usually via intravenous injection in an antecubital vein to enhance the cardiac and/or coronary anatomy during imaging. According to the Society of Cardiovascular Computed Tomography Guidelines for the performance and acquisition of CCTA as described by Abbara et al. in “SCCT guidelines for the performance and acquisition of coronary computed tomographic angiography: A report of the Society of Cardiovascular Computed Tomography Guidelines Committee Endorsed by the North American Society for Cardiovascular Imaging (NASCI)”, J Cardiovasc Comput Tomogr. 2016 November-December; 10(6):435-449, the contrast medium injection is timed in such a way that the coronary arterial system contains sufficient contrast medium to clearly distinguish the coronary artery lumen from surrounding soft tissues. This enables the physician assessment of luminal narrowing as well as coronary artery stenosis with an optimal image quality and thus accuracy. To ensure adequate coronary artery opacification, the guidelines describe that CCTA image acquisition is typically started once a pre-defined threshold attenuation value has been reached in a pre-defined anatomical structure (most often this concerns the descending aorta), or by waiting a certain delay time after enhancement is first visible in the ascending aorta. Furthermore, the CT imaging operations can be triggered by analysis of an ECG signal of the patient.

In step 202, the processors extract data representing an axial trajectory extending along the vessel of interest. For example, the axial trajectory may correspond to a centerline extending along the vessel of interest. When the vessel of interest represents the coronary artery, the axial trajectory may correspond to the coronary centerline, in which the processors extract the coronary centerline. The coronary centerline represents the center of the coronary lumen along the coronary section of interest. This can be a single coronary artery, a coronary bifurcation, or the full coronary tree. In case when the coronary section of interest includes one or more bifurcation(s), the coronary centerline will include bifurcation(s) as well but not its side branch.

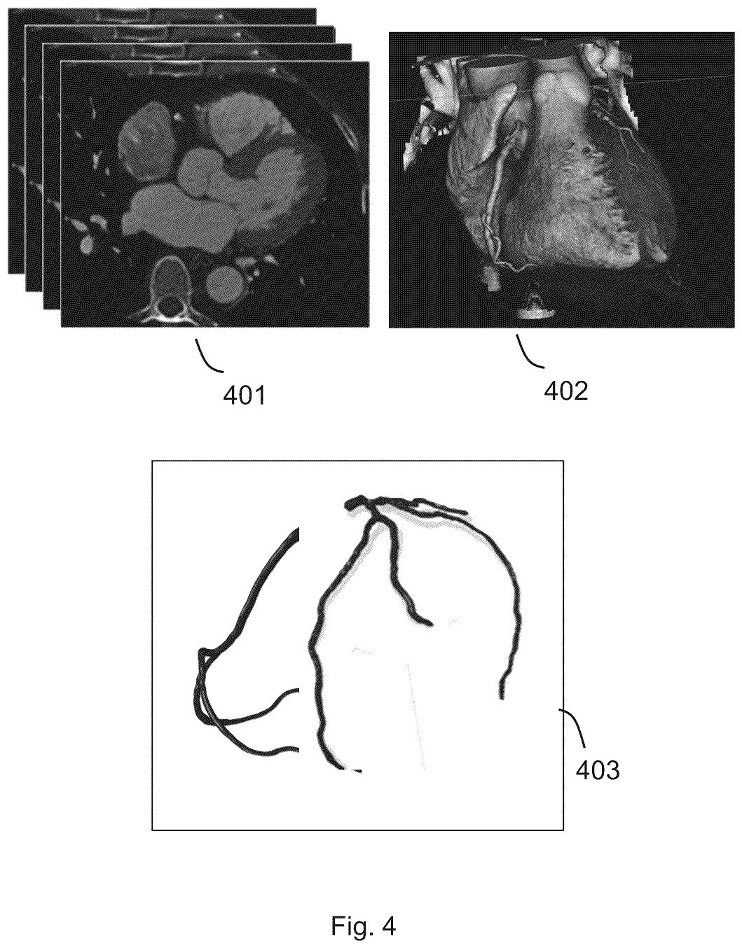

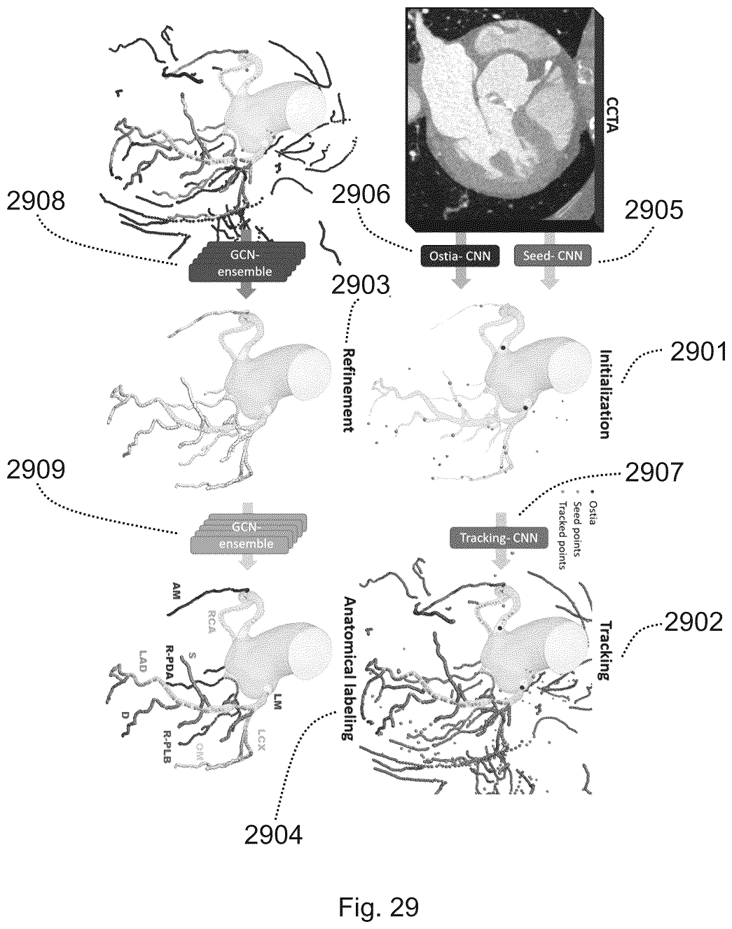

In the case that a bifurcation and/or the coronary tree is analyzed, data representing multiple centerlines can be extracted in step 202. For the purpose of current application, it is not required that the extracted coronary centerline represents the center of the coronary lumen accurately. A rough estimation of the coronary centerline is sufficient, although the coronary centerline should not exceed the coronary lumen. The extraction of the coronary centerline can be performed manually or (semi)automatically. An example of a semiautomatic approach is described by Metz et al., “Semi-automatic coronary artery centerline extraction in computed tomography angiography data”, proceedings/IEEE International Symposium on Biomedical Imaging: from nano to macro, May 2007. An example of an automatic coronary centerline extraction method is described by Wolterink et al. in which machine learning is utilized to automatically extract the coronary centerline in “Coronary artery centerline extraction in cardiac CT angiography using a CNN-based orientation classifier”, Med Image Anal. 2019 January; 51:46-60. The method extracts, after placement of a single seed point in the artery of interest, the coronary centerline between the ostium and the most distal point as visualized in the CCTA image dataset. In a preferred embodiment, the complete coronary centerline tree is automatically extracted, and each coronary segment is automatically labelled for instance according to the model introduced by the American Heart Association (Austen et al, “A reporting system on patients evaluated for coronary artery disease. Report of the Ad Hoc Committee for Grading of Coronary Artery Disease, Council on Cardiovascular Surgery, American Heart Association”, Circulation 51, 5-40. 1975). This method is further described with reference to the flowchart of FIG. 28 and its high-level methodology as illustrated in FIG. 29. The vessel of interest can be identified by the user, or predefined for instance the left anterior descending artery (LAD), left Circumflex artery (LCx), or right coronary artery (RCA), or automatically by the method discussed with reference to the flowchart of FIG. 28. FIG. 4 shows an example of an extracted centerline tree (403) from a CCTA dataset (401). A CCTA dataset consists of several 2D images (401), resulting in an acquired volume which is represented by volumetric rendering of this acquired volume by 402. In case of multiple arteries, the method as described by FIG. 2 or FIG. 17 or FIG. 23 can be automatically performed on multiple vessels of interest, for instance on the LAD, LCx and RCA. These multiple vessels of interest can be predefined and automatically extracted by the method as described by FIG. 28.

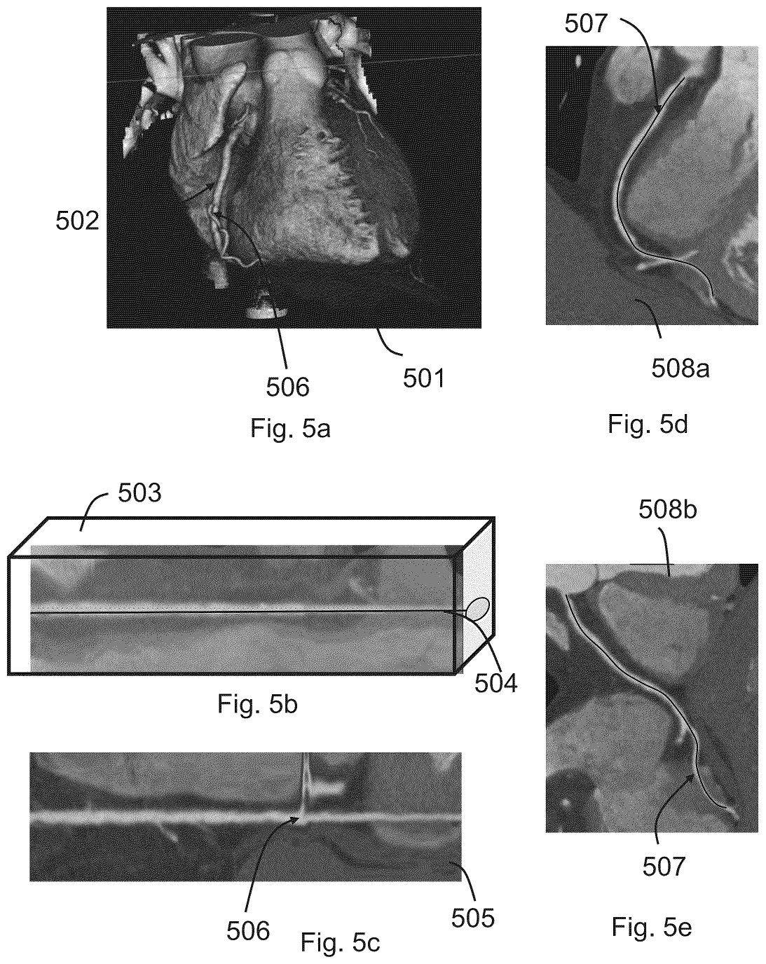

In step 203, the data representing the axial trajectory (or centerline(s)) extending along the vessel of interest as extracted in step 202 is used to create a three-dimensional (3D) multi-planar reformatted (MPR) image of the coronary artery of interest. FIGS. 5a-5d provide an illustration of the creation of the volumetric 3D MPR image. Image 501 of FIG. 5a shows a volumetric rendering of a CCTA dataset (FIG. 2, 201), in which the right coronary artery 502 is selected as an example to create a 3D MPR image. With respect to the 3D MPR image, there is a distinction between straight MPR and curved MPR. For the straight MPR as well for the curved MPR, the extracted axial trajectory (e.g., centerline) is used to create an isotropic 3D MPR image from the image dataset 201. The resolution of the 3D MPR image is predefined and is for example using for instance trilinear interpolation. The 3D MPR image can also be created in a non-isotropic way, for instance 0.1 mm in-plane pixel size and 0.25 mm distance between consecutive centerline points using for instance trilinear interpolation.

The creation of the 3D MPR image can involve sampling the image data along the extracted axial trajectory 502 (e.g., coronary centerline) to define a cuboid image 503 in such a way that the coronary centerline is in the center of the cuboid image 504, resulting in a straight MPR. Image 503 of FIG. 5b illustrates the cuboid resampled image (straight MPR) and one ‘slice’ is visualized within the cuboid resampled image for easy interpretation. Image 505 of FIG. 5c shows a one ‘slice’ of the same resampled image, but the visualization plane is rotated around the centerline 504, to illustrate the visualization of the coronary bifurcation (506) within the extracted right coronary artery.

Alternatively, the creation of the 3D MPR image can involve sampling the image along the curved course of axial trajectory 502 (e.g., coronary centerline), resulting in a curved MPR. Images 508a and 508b of FIGS. 5d and 5e show two examples of a curved 3D MPR image and visualized as a single ‘slice’, in which the slice orientation refers to a curved plane which can be rotated along the curved coronary artery. Again, this is just for visualization purpose, the application will use the full 3D straight or curved MPR image. The advantage of a curved MPR image is that the curvature or tortuosity of the extracted centerline can be taken into account within the described machine learning network architectures within this application.

In alternate embodiments, the MPR image created in step 203 can be represented by a two-dimensional (or “2D”) MPR image of the vessel of interest.

In step 204, the MPR image of step 203 is supplied to a machine learning based artery characterization network that extracts data (signals) that characterize features of the vessel of interest along the centerline of the vessel of interest given the MPR image as input.

In embodiments, machine learning based artery characterization network of step 204 employs a convolutional neural network (CNN) architecture. The CNN architecture typically includes an input layer, hidden layers, and an output layer. The hidden layers include one or more layers that perform convolutions. Typically, this includes a layer that performs a dot product of the convolution kernel with the layer's input matrix. This product is usually the Frobenius inner product, and its activation function is commonly a rectified linear unit (ReLU). As the convolution kernel slides along the input matrix for the layer, the convolution operation generates a feature map, which in turn contributes to the input of the next layer. This is followed by other layers such as pooling layers, fully connected layers, and normalization layers.

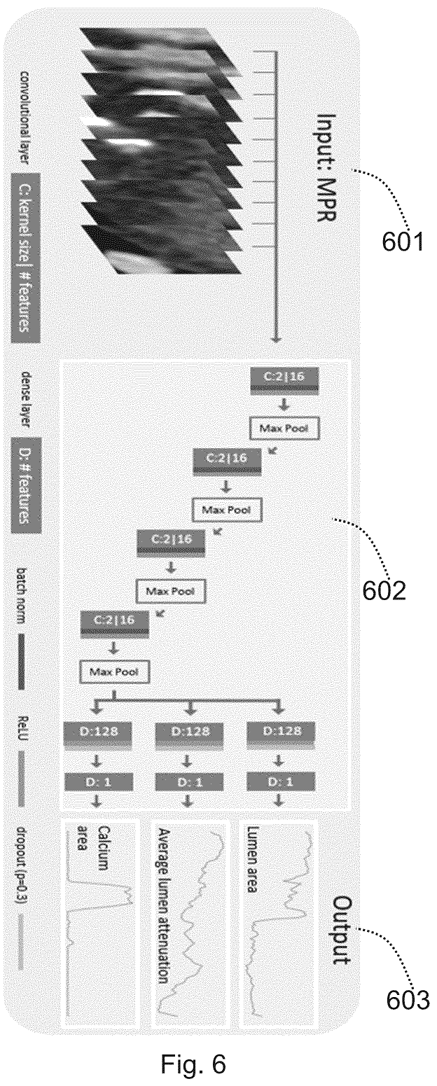

FIG. 6 shows an example CNN architecture for extracting the artery characteristics. For each point along the centerline of the vessel of interest, the artery characterization network predicts, based on the MPR image (601), a number of artery characteristics relevant for artery lesion severity. In the artery characterization network as illustrated by FIG. 6, three characteristics are extracted by the CNN architecture. These characteristics or features include cross-sectional lumen area, the lumen attenuation (optionally) and calcium area, all specified along the axial trajectory of the vessel of interest. An example of the CNN architecture is provided by 602 in FIG. 6, which analyses stacks of a predefine amount of successive cross-sectional slices within the MPR image (for instance 5 cross-sections) and consists of for instance four alternating convolutional blocks and pooling operations. Convolutional blocks are comprised of two convolutional layers (for example with kernel size 3, 16 filters), each followed by batch normalization and the rectified linear unit (ReLU) activation function. Finally, three separate output heads regress values for the lumen area, average attenuation in the lumen and calcium area for the central slice of the input stack are provided, resulting in output artery characteristics (in current example, lumen area, lumen attenuation and calcium area) along the axial trajectory (e.g., centerline) of the vessel of interest (FIG. 6, 603).

To train the artery characterization network (204 of FIG. 2, or the CNN of FIG. 6), artery reference values are used (FIG. 2, 208). The artery reference values (208) are provided by a database which contains data of multiple patients. Each set within the database contains for each patient a) contrast enhanced CT image datasets (201 represents reference image sets during the training phase) and corresponding b) artery characteristics reference values. For the three characteristics as described above, reference annotations of the coronary artery lumen and coronary calcium are required from which cross-section luminal area, calcified area, and lumen attenuation along the axial trajectory of the vessel of interest is derived. These reference annotations can be created by manual segmentation of CCTA datasets used during the training, or by automatic segmentations of the lumen and calcium of CCTA datasets used during the training followed by manual corrections when needed. Automatic segmentation of the lumen and calcium can for instance be performed by segmented in the original CT image volumes using the approach as described by Wolterink et al, “Automatic coronary artery calcium scoring in cardiac CT angiography using paired convolutional neural networks”, Medical Image Analysis 34, 123-136 (2016) and Wolterink et al, “Graph Convolutional Networks for Coronary Artery Segmentation in Cardiac CT Angiography”. In: Zhang D, Zhou L, Jie B, Liu M, editors. Graph Learning in Medical Imaging. Lecture Notes in Computer Science. Cham: Springer International Publishing (2019). p. 62-9. Thereafter, automatic segmentations were transferred to the MPR of the artery, visually inspected and corrected when needed. As X-ray angiography is superior in image resolution as compared to CCTA, within a preferred embodiment the lumen segmentation on MPR image is guided by QCA3D analysis results extracted from X-ray angiographic image data from the same patient which is acquired within three months of the acquisition of the CCTA data of the patient in question. The QCA3D can be performed for instance by CAAS Workstation (Pie Medical Imaging BV, the Netherlands), using the approach as described by Girasis C, et al, “Advanced three-dimensional quantitative coronary angiographic assessment of bifurcation lesions: methodology and phantom validation”, EuroIntervention 2013; 8: 1451-1460.

To ensure that the artery reference values (208) are aligned to the spatial coordinates of the MPR image (203), the artery reference values can be transformed to the MPR image domain. In case the artery reference values (e.g., manual annotation of plaque type, functional lesion severity such as for instance FFR) are obtained by using the MPR image as a result from step 203, this step may be skipped. When the artery reference values are obtained, for instance by annotation using the contrast enhanced CT datasets (step 201), this step transforms the annotation into the MPR view. Such a transformation is performed by using the extracted centerline as a result of step 202. Transformation of the coronary tree characteristics (205) can be performed according to the above description.

To ensure that the fractional flow reserve values along the coronary artery as measured in the catheterization laboratory (e.g., pullback FFR reference values) are aligned to the spatial coordinates of the MPR image, a co-registration is performed between the image dataset 201 and the invasively measured pullback FFR. To allow co-registration of the pullback FFR measurement with the CT dataset, pullback motion information is obtained indicative of the pullback rate or speed during withdrawal of the FFR wire from an FFR wire start location (e.g., distal position in the coronary artery) to an FFR wire end location (e.g., proximal position in the coronary artery or the ostium of the coronary artery). The pullback motion information can be obtained by measuring the longitudinal motion of the FFR wire during pullback. The measurement may be obtained in various manners, such as by means of a motion measurement system, or for instance by utilizing a motorized pullback device that maintains a constant pullback speed. The one or more processors of the system utilize the time required to pullback the FFR wire and the pullback speed to calculate a length of a pullback distance. In order to align the pullback FFR artery reference values into the MPR image, the one or more processors transform the length of the pullback distance to the image dataset used 703.

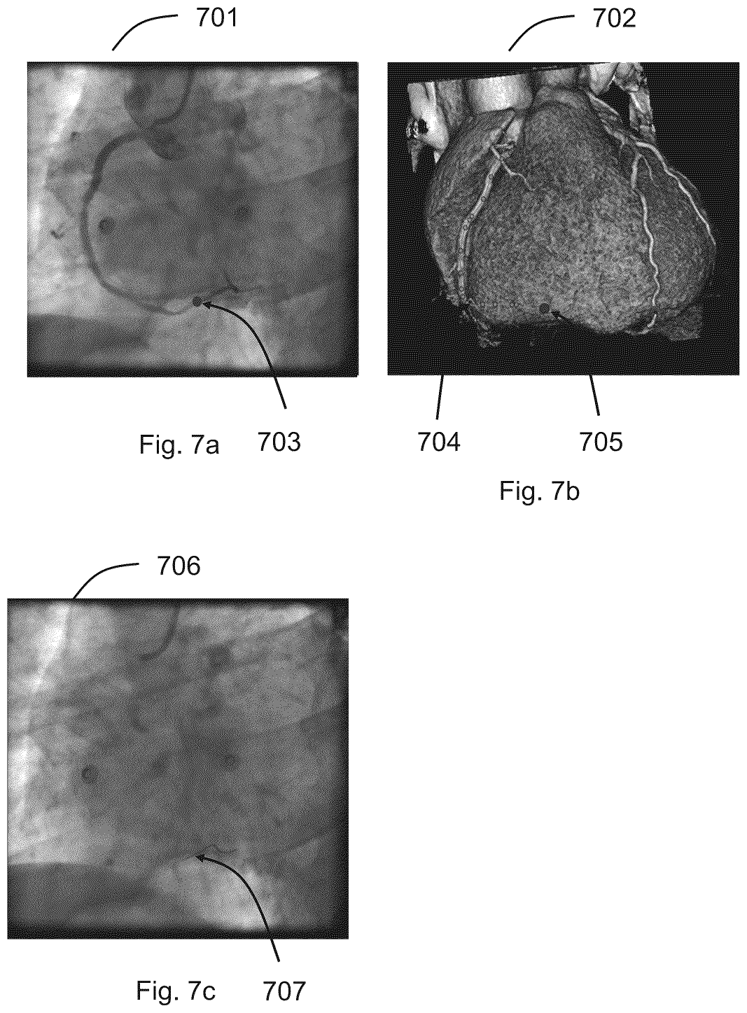

FIGS. 7a-7c provides a schematic illustration of a co-registration method of a pullback FFR reference value (e.g., pullback distance) with the CCTA image dataset. Image 701 of FIG. 7a shows an x-ray coronary angiographic image as acquired within the catheterization laboratory. Image 702 of FIG. 7b shows a volume rendered CCTA image belonging to the same patient. Image 706 of FIG. 7c shows an x-ray fluoroscopic image without contrast liquid present.

The x-ray coronary angiographic image 701 of FIG. 7a illustrates the right coronary artery with an FFR pressure wire inserted therein to a desired distal position at which a pressure sensor may obtain first/distal pressure measurements of interest. The dot 703 indicates a location of the pressure sensor on the FFR pressure wire when located at the distal position within the coronary artery before pullback on the x-ray angiographic image. The position denoted by dot 703 may also be referred to as a distal pressure sensor position. The distal position of the pressure sensor (and entire FFR pressure wire) is easily identifiable on x-ray fluoroscopic image (without contrast liquid present, 706) due to the radiopaque marker on the FFR wire 707 (as shown in the image 706 of FIG. 7c) enabling to localize the pressure sensor on the FFR wire.

Image 702 of FIG. 7b shows a volume rendered CCTA image (belonging to the same patient in which the pullback FFR reference values are obtained). In image 702, the right coronary artery 704 is identified for instance as a result from step 202 of FIG. 2. Co-registration of the pullback FFR reference values can be performed by identifying the location of the FFR pressure wire before pullback within the CCTA dataset (705), for instance manually identifying supported by anatomical landmarks such as bifurcation location, and align the FFR values by matching the lengths (length of the 3D extracted centerline with the length of the FFR pullback). The identification of the location of the FFR pressure wire before pullback within the CCTA dataset can also be performed by registration of the x-ray angiographic image with the CCTA dataset, as for instance by using the method of Baka et al. “Oriented Gaussian Mixture Models for Nonrigid 2D/3D Coronary Artery Registration”, IEEE Trans Med Imaging. 2014 May; 33(5):1023-34. Baka et al describes a method to register a 2D x-ray angiographic image to a 3D volumetric image dataset (CCTA) by using a Gaussian mixture model (GMM) based point-set registration technique. Since the location of the pressure sensor (707) on the FFR wire can be easily performed using x-ray fluoroscopic image (706) by means of image processing techniques, the transformation of this location into the CCTA image data (705) is straightforward using the deformation field resulting from the 2D/3D registration as described by Baka et al.

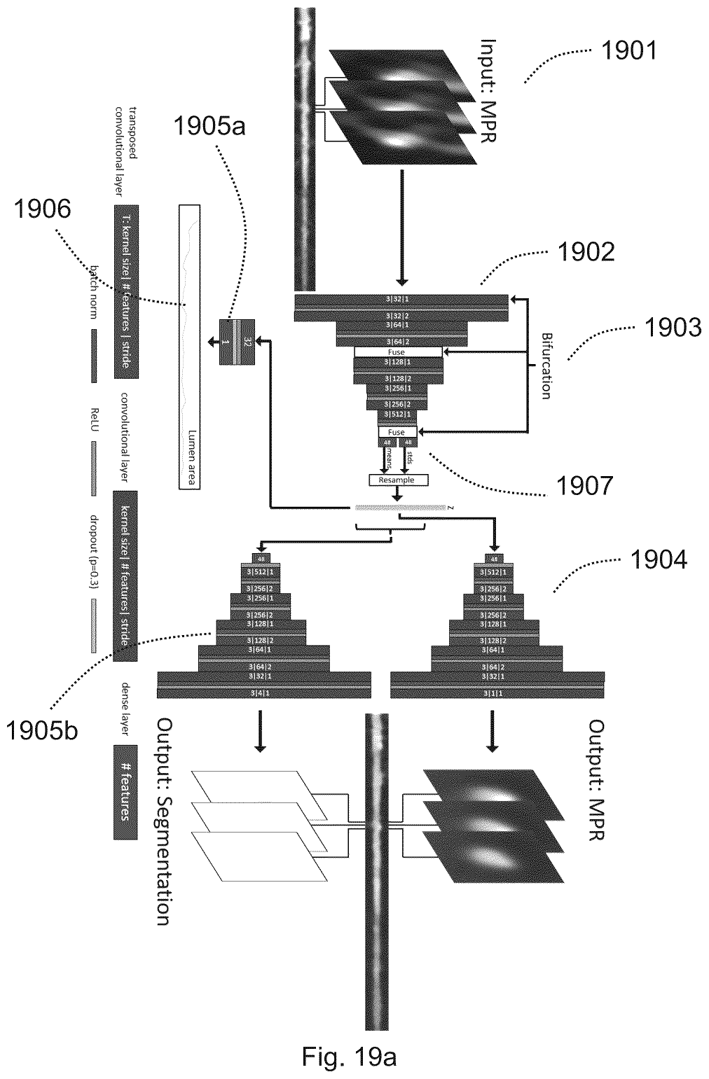

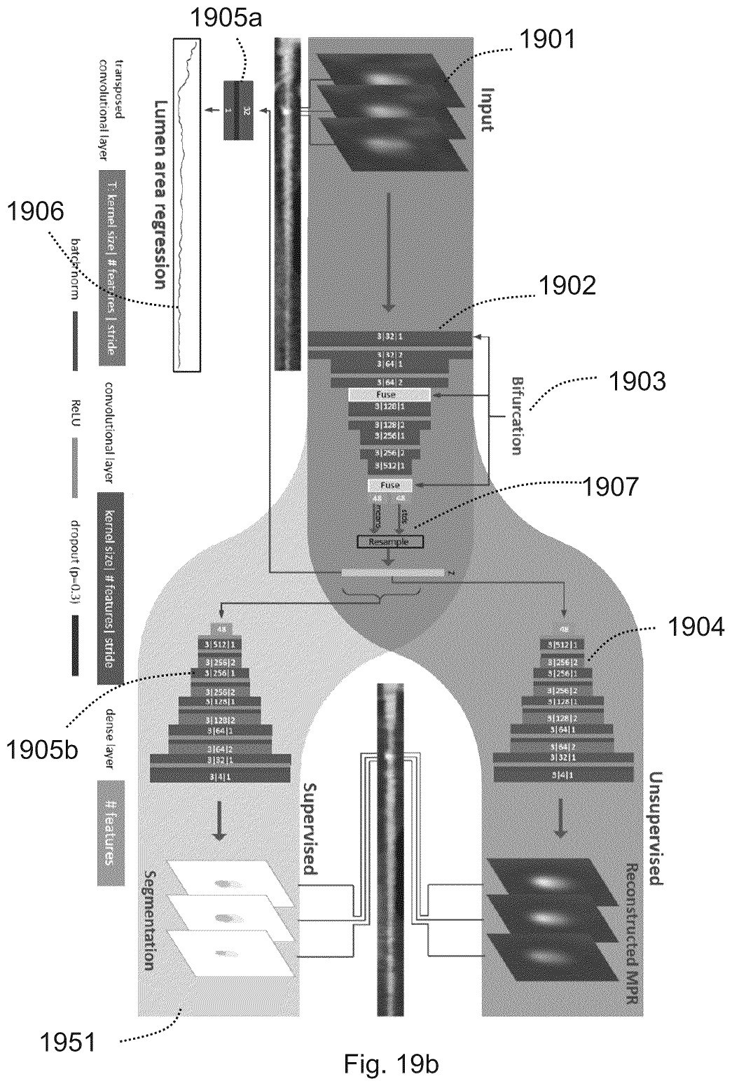

In other embodiments, the machine learning based artery characterization network of step 204 can employ a variational autoencoder (VAE) architecture configured to extract features or characteristics of a vessel of interest given an MPR image of the vessel of interest as input. Details of the variational autoencoder (VAE) architecture are described below with respect to step 1704 of FIGS. 17 and FIGS. 19a and 19b.

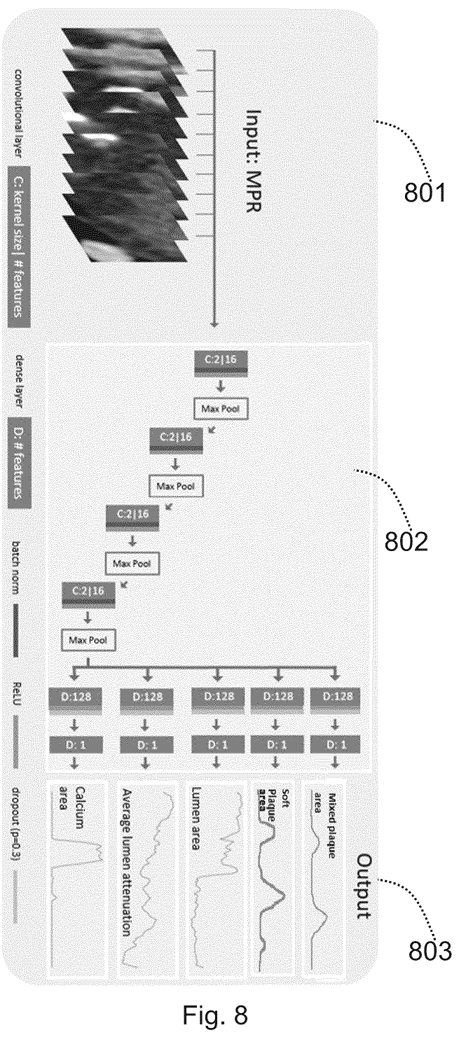

In yet other embodiments, the machine learning based artery characterization network of step 204 can be configured to extract other data (signals) that characterize features of the vessel of interest given the MPR image as input. This can be achieved by including multiple artery characteristics in the artery characterization. For example, FIG. 8 shows an example CNN network architecture for extracting five artery characteristics along the centerline of the vessel of interest, based on the MPR image (801). The five artery characteristics include cross-sectional lumen area, the lumen attenuation, the calcium area, soft plaque area and mixed plaque area. During training of the network as illustrated by FIG. 8, two additional reference features need to be extracted from the annotation (soft plaque and mixed plaque) as compared to the network as illustrated and described by FIG. 6. This annotation can be based on manual or (semi)automatic segmentation in the CCTA image dataset. Automatic plaque segmentation can be based on for instance the approach described by Javorszky et al, “Deep learning-based atherosclerotic coronary plaque segmentation on coronary CT angiography”, European radiology vol. 32,10 (2022).

An example of this CNN architecture is provided by 802 in FIG. 8, which analyses stacks of a predefine amount of successive cross-sectional slices (for instance 5 cross-sections) and consists of for instance four alternating convolutional blocks and pooling operations. Convolutional blocks are comprised of two convolutional layers (for example with kernel size 3, 16 filters), each followed by batch normalization and the ReLU activation function. Finally, five separate output heads regress values for the lumen area, average attenuation in the lumen, calcium area, soft plaque area and mixed plaque area for the central slice of the input stack are provided, resulting in output artery characteristics (in current example, lumen area, lumen attenuation, calcium area, soft plaque area and mixed plaque area) along the centerline of the vessel of interest (FIG. 8, 803).

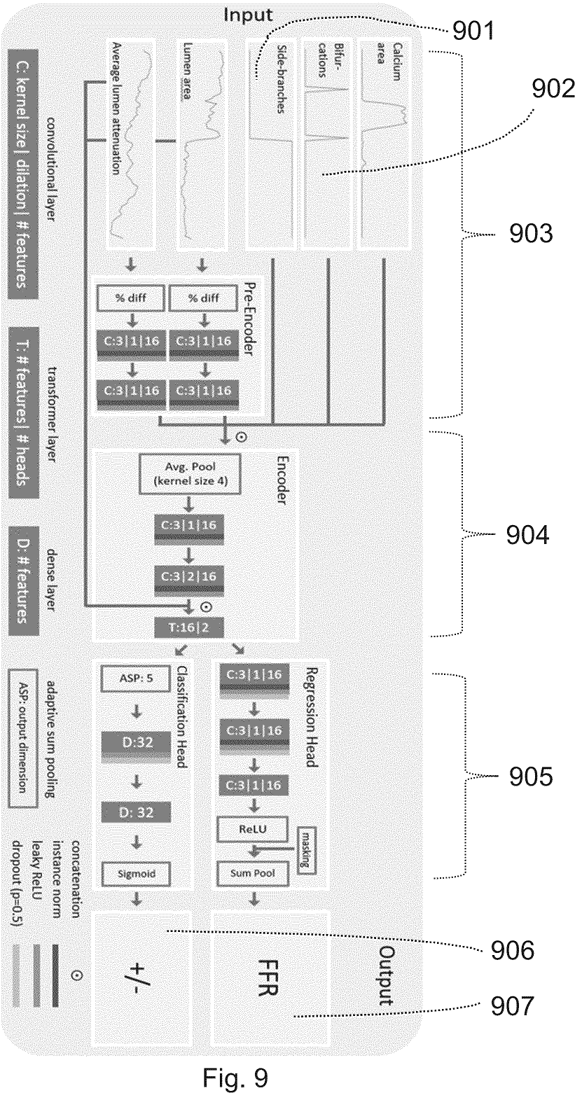

Vessel geometry has an impact on the characteristics of the blood flow and local appearance of the vessel. Therefore, in step 205 of FIG. 2, other data (signals) that characterize features of the vessel of interest along the axial trajectory of the vessel of interest (as obtained from step 202 of FIG. 2) can extracted from the CCTA image data of step 201 of FIG. 2. For example, for each point along the axial trajectory (i.e., centerline) of the vessel of interest, two additional characteristics can be extracted from the CCTA image data. The first one indicates the presence of bifurcations at the artery centerline point (901 of FIG. 9). As described by step 202 of FIG. 2, the coronary centerline for the vessel of interest can be performed manually or (semi)automatically. In a preferred embodiment, the complete coronary centerline tree is automatically extracted using the methodology as illustrated in FIG. 29. When using this method, the presence and location of bifurcations along the centerline of the artery of interest can be automatically determined by identifying the centerline points along the centerline of the artery of interest in which a branching artery is present. Alternatively, the user can manually identify branching arteries (bifurcation) for instance in the CCTA slice (401 of FIG. 4), or the volume rendered CCTA image (402 of FIG. 4). In yet another alternative method is the train a deep learning network on the MPR image that automatically identifies branching arteries (bifurcation). The second additional characteristic indicates whether a centerline point belongs to a main branch (i.e., left main (LM), LAD, LCX, RCA) or a side-branch (902 of FIG. 9). This can be automatically extracted for instance as described by step 202 of FIG. 2 using the automatic anatomical labeling methodology as illustrated by FIG. 29. Furthermore, the vessel of interest can be identified or selected by the user. If the vessel of interest includes a side-branch or bifurcation, the user can identify the position of the side branch or bifurcation. These characteristics can be normalized to zero mean and unit variance across the training data set. Examples of additional characteristics that characterize the vessel of interest could be the presence of a stent and/or type of stent (from a previous coronary intervention procedure) at the vessel centerline point.

In step 206, a machine learning based stenosis assessment network is configured to assess functional significance of stenosis given the feature data output by the first network (204 of FIG. 2 or the CNN of FIG. 6 or 8) as well as the other feature data extracted from the CCTA image data (205 of FIG. 2) as input.

In embodiments, the machine learning based stenosis assessment network can utilize a CNN network architecture as described herein. FIG. 9 shows an example CNN network architecture for the stenosis assessment. At a high level, the CNN architecture of FIG. 9 consists of three stages. In the first stage (903), the lumen area and its attenuation predicted by the characterization network (204 of FIG. 2) are first pre-encoded and subsequently concatenated with the calcium area, and with additional characteristics indicating bifurcations and whether the analysis is performed in the main- or side-branch of the artery as a result of step 205, FIG. 2. In the second stage (904), the combined encodings are fed to an encoder. In the encoder, the features are first pooled and thereafter, convolutions and a transformer layer are applied. For final classification, in the third phase (905), two separate output heads (regression head and classification head) are applied. In the regression head, the output of the second stage (904) is processed by two convolutional layers and a ReLU activation function. The resulting sequence is pooled along the artery dimension and subtracted from 1 to yield a single FFR value. In the classification head, the output of the second stage (904) is pooled to a fixed length of for instance 2.5 mm. Thereafter, two dense layers are used in combination with the sigmoid activation function to yield output probabilities for the presence of a functionally significant stenosis in the artery.

The three stages of the machine learning based stenosis assessment network of FIG. 9 are described in detail below.

In the first stage (903), the network receives the five artery characteristics (lumen area, average lumen attenuation (optional), calcium area, bifurcations, and side-branches) as input. To focus on changes in the lumen area and its attenuation rather than their absolute values, the percentage difference at each location in the artery with respect to the previous location is calculated. Because the relevant features in the lumen area and its attenuation may be subtle and may appear in different locations along the artery (i.e., a stenosis is expected to cause changes in the attenuation distal to the appearance in the lumen area), these two characteristics are first separately encoded. This is done using two non-shared convolutional layers with the Leaky Rectified Linear Unit (LeakyReLU) activation function applied in between the layers. Thereafter, the remaining characteristics are concatenated with the encoded features from the lumen area and its attenuation.

In the second stage (904), the information of all five extracted artery characteristics is merged by a common encoder, consisting of convolutional layers and a transformer layer, as follows: To increase the receptive field and reduce the dimensionality, average pooling with kernel size of for example 4 is applied, followed by two convolutional layers with for example dilation 1 and 2, respectively. Each convolutional layer is followed by the LeakyReLU activation function, instance normalization and dropout. Subsequently, artery encodings are concatenated with the original lumen area and its attenuation, and fed to a transformer layer (Vaswani et al, “Attention is All you Need”, Advances in Neural Information Processing Systems. Vol. 30. Curran Associates Inc. 2017). Due to the global receptive field, the transformer layer connects all artery points with one another. This potentially enables modeling interaction between multiple lesions, and proximal and distal section of the artery.

In the third stage (905), two separate output heads (regression head and classification head) are configured to perform separate tasks: the regression head performs regression of the FFR value, and the classification head performs classification of the presence of a functionally significant stenosis in the artery. Inspired by the additive nature of sequential flow resistances, the regression head is designed to predict pressure drops along the artery. First, two layers of convolutions are applied, each followed by the LeakyReLU activation function, instance normalization and dropout. Thereafter, a third convolutional layer with a single output filter map is followed by a ReLU activation function to enforce positivity of the pressure drops. Finally, the predicted pressure drops are summed up along the artery using a sum pooling layer and the resulting overall FFR drop is transformed into the final FFR value (907) by subtracting it from 1. The classification head output (906) predicts the presence of functionally significant stenosis (FFR≤0.8). To explicitly relate proximal and distal sections, first, adaptive sum pooling with for example 5 output features is applied followed by for example two dense layers, each with LeakyReLU activation and dropout. Finally, a dense layer with a single output filter map and sigmoid activation yields output probabilities for functionally significant stenosis.

In embodiments, all convolutions throughout the stenosis assessment network of FIG. 9 can employ a kernel size of for example 3 in combination with zero-padding to prevent shrinkage of the features. Furthermore, for all convolutions as well as for the transformer, a relatively small number of for example 16 filter maps is utilized, to balance the required expressiveness and to prevent overfitting. For the same purpose, all dropout probabilities can be set for example to 0.5.

To train the stenosis assessment network (206 FIG. 2 of the CNN of FIG. 9), a reference standard can be used (FIG. 2, 209). The reference standard is a database which contains data of multiple patients. Each set within the database contains for each patient a) contrast enhanced CT image datasets (201 represents reference image sets during the training phase) and corresponding b) CAD related reference values representing a hemodynamic index for assessment of functionally significant coronary artery obstruction. For example, the CAD related reference values may represent at least one invasively measured fractional flow reserve, coronary flow reserve, instantaneous wave-free ratio, resting full-cycle ratio, diastolic hyperemia free ratio, diastolic pressure ratio, resting Pd/Pa ratio, hyperemic myocardium perfusion, index of microcirculatory resistance, pressure drop along a coronary artery and the fractional flow reserve along a coronary artery. Note that the reference standard (209) and the artery reference values (208) require the same contrast enhanced CT image datasets.

During training, the regression head is supervised using the mean squared error with for example the CAD reference value FFR. Since the invasive reference FFR is often not measured at the most distal location, predicted pressure drop contributions from anatomical locations distal to the measurement location are masked during training and testing. The measurement location is assumed to be for example 10 mm distal to the annotated lesion location, in line with measurement protocols from clinical practice. The classification task is supervised using the binary cross entropy loss function. The loss terms of the regression head and the classification head are weighted equally.

Finally, in step 207, one or more outputs are provided. In embodiments, the outputs represent a probability for the presence of a functionally significant stenosis in the vessel of interest. In yet another embodiment the outputs represent the FFR as a value between 0.0 and 1.0. To combine strengths of the results from the classification head (906) and the results of the regression head (907), their outputs are merged into a single probability for the presence of a functionally significant stenosis in the vessel of interest. While the classification head directly predicts probabilities for the positive and negative class, the regressed FFR values are distributed around the threshold of positive FFR (≤0.8) and in the range [0.0, 1.0]. To allow their merging, the predicted FFR values are first transformed into pseudo-probabilities by linearly scaling a symmetric window around the positive FFR threshold of 0.8, using the formula of equation 1:

$${p}_{pseudo} = \{ \begin{matrix}0.5 - \frac{( {FFR}_{regress} - 0.8 )}{0.4} & for {FFR}_{regress} ∈ [ 0.6 , 1. ] \\ 1. & for {FFR}_{regress} ∈ [ 0. , 0.6 )\end{matrix} \qquad ( equation 1 )$$

To obtain the final prediction result of the output, the pseudo-probabilities can be averaged with the probabilities from the classification head.

Optionally, to increase robustness of the prediction result and to determine the uncertainty of the prediction result, the output of step 207 can be calculated as an averaged over multiple trained networks (both 204 and 205). For instance, by performing a randomized tenfold cross-validation, in which ten networks are trained on random 90% subsets and testing on the remaining 10%. During testing we ensemble the networks by averaging the predicted probabilities and FFR values as for instance thought by Müller et al, “An analysis on ensemble learning optimized medical image classification with deep convolutional neural networks”, in IEEE Access, vol. 10, pp. 66467-66480, 2022. For the prediction of the uncertainty, the standard deviation is calculated over the probabilities and the FFR values as for instance thought by Lakshminarayanan et al, “Simple and scalable predictive uncertainty estimation using deep ensembles”, Advances in Neural Information Processing Systems. Vol. 30. Curran Associates Inc (2017). The uncertainty measure may be valuable in clinical practice where the method could be employed in a semi-automatic setting by introducing for instance a hybrid approach. In particular, patients with arteries in which the method indicates high prediction uncertainty could be referred for invasive measurements.

### Experimental Settings

In embodiments, the artery characterization network (204 of FIG. 2, or the CNN of FIG. 6) was trained for 800 epochs using the mean absolute error as loss function and the ADAMW (Loshchilov and Huffer F, “Decoupled weight decay regularization”, International Conference on Learning Representations-ICLR 2019) optimizer with a learning rate of 10⁻⁵ and a batch size of 512. The loss term of the lumen attenuation was scaled with a factor of 0.1. After training, the network was applied to each cross-section of the MPR to obtain the lumen area, its average attenuation, and the area of calcium along the length of the artery.

In embodiments, the stenosis assessment network (206 of FIG. 2, or the CNN of FIG. 9) was trained for 150 epochs using the ADAMW optimizer with a linearly scheduled cyclic learning rate. The cyclic learning rate varied between 5e-4 and 1e-5 over a period of 40 epochs. Because different artery lengths limit the network for stenosis assessment to process only a single artery at a time, the loss was accumulated over 8 training iterations before backpropagating, corresponding to an effective batch size of 8.

### Extensions to the Flowchart of FIG. 2.

This section describes several extensions to the flowchart of a machine learning based method for determining functionally significant lesion severity in one or more coronary arteries as described before with reference to FIG. 2.

### Extension 1: FFR Value Per Centerline Point.

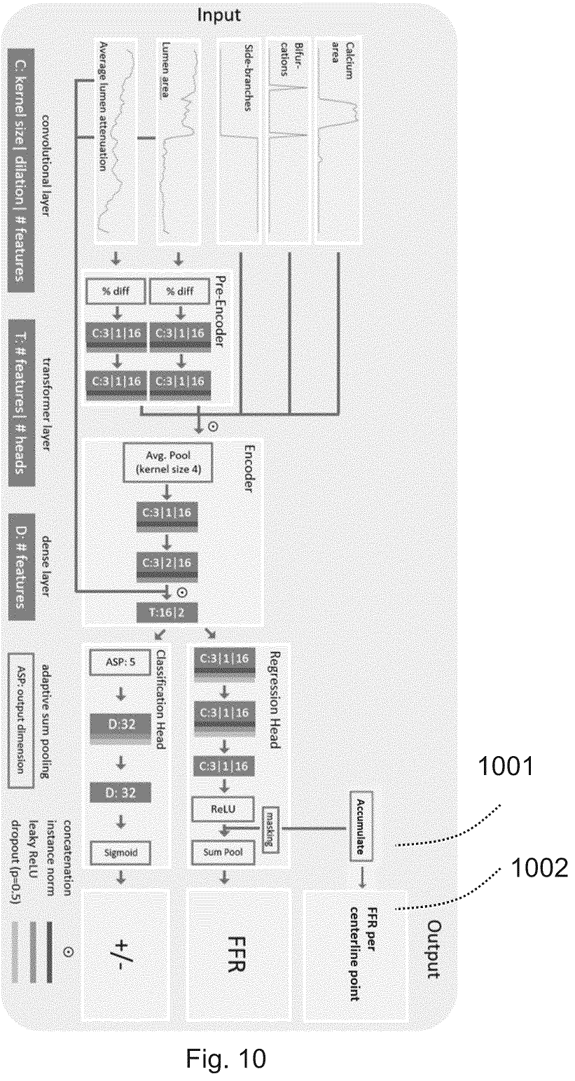

The machine learning based stenosis assessment network as described by 206 of FIG. 2, and further clarified with respect to FIG. 9, can be configured to provide two outputs: a regressed FFR value for the entire vessel of interest and a binary classification of the presence of a functionally significant obstruction of blood flow in the vessel of interest. This section describes an extension to the stenosis assessment network which can be implemented by module 304 of FIG. 3. In particular, the architecture of the deep learning networks can be adapted to provide regressed FFR values for centerline points along the vessel of interest, resulting in a FFR values at centerline points of the vessel of interest (FFR pullback graph). FIG. 10 shows the architecture of this extension based on the stenosis assessment network architecture as presented by FIG. 9.

With respect to the description of the stenosis assessment network (206 of FIG. 2, and further clarified with respect to FIG. 9), the regression head is extended to include the FFR per centerline point output. All other methodology is identical to the description of the flowchart from FIG. 2. This extension is focused on the regression head which is part of the stenosis assessment network (FIG. 9). The regression head is designed to predict pressure drops along the artery. First, two layers of convolutions are applied, each followed by the LeakyReLU activation function, instance normalization and dropout. Thereafter, a third convolutional layer with a single output filter map is followed by a ReLU activation function to enforce the positivity of the pressure drops, which corresponds to predicting the FFR drop of each location or point along the centerline of the vessel of interest (given the multi-planer reconstruction (MPR) view as depicted in FIG. 6, 601, and FIG. 8, 801) and performed by the ‘Accumulate’ block as shown in FIG. 1001. This results in an FFR drop per centerline point along the vessel of interest and can be transformed into an FFR value per centerline point by subtracting it from 1.0 (FIG. 1002), resulting in an FFR pullback graph. The FFR value per centerline point of the vessel of interest can be presented visually to a user. For example, the FFR value per centerline point can be plotted (depicted visually) along the vessel of interest in an image of the vessel of interest. In another example, the FFR value per centerline point can be plotted (depicted visually) in a CCTA volume rendering of the vessel of interest. In yet another example, the FFR value per centerline point can be plotted (depicted visually) in a 3D model of the vessel of interest. In still another example, the FFR value per centerline can be plotted (depicted visually) by color coding a 3D representation of the vessel of interest (either in the CCTA volume render view, or in a 3D segmentation) representing the vessel of interest.

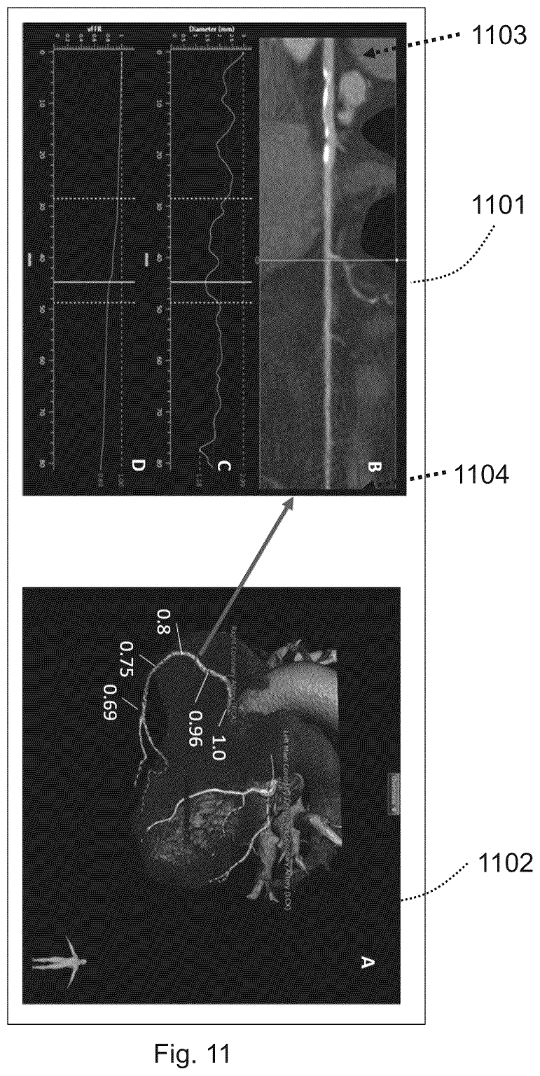

FIG. 11 shows an exemplary graphical user interface (display screens) that visually conveys such information to a user. In FIG. 11, 1102 shows a volume-rendered image of the CCTA data in which the three main coronary arteries are enhanced; right coronary artery (RCA), left anterior descending coronary artery (LAD), and the left circumflex coronary artery (LCX). The RCA is the vessel of interest, and results in a multiplanar reconstruction (MPR) illustrated by 1101-part B of FIG. 11 in which the left side (1103) corresponds to the ostium (proximal) location of the RCA and right side (1104) corresponds to the distal location of the RCA. 1101-part C shows the lumen area or lumen diameter graph along the RCA, in which the x-axis corresponds to the x-axis of the MPR view of 1101 part B and the centerline of the RCA. Finally, 1101-part D shows the FFR values along centerline points in the RCA, in which the x-axis is identical to the x-axis of the MPR view and the diameter/area graph. Furthermore, in 1102, the FFR values along the centerline of the volume-rendered RCA are used to color code the corresponding segments of the volume-rendered RCA. Note that the vertical markers in 1101-parts C, D correspond to the minimum area/diameter (thick line markers) and the obstruction extent (dash markers) derived from common quantitative coronary analysis techniques as for instance described by Girasis C, et al, “Advanced three-dimensional quantitative coronary angiographic assessment of bifurcation lesions: methodology and phantom validation”, EuroIntervention 2013; 8: 1451-1460.

For training the stenosis assessment network illustrated by FIG. 10 to output an FFR value per centerline point, reference FFR values (209) can be measured along a coronary vessel of interest, resulting in an invasively measured fractional flow reserve value at each position along the coronary centerline. This can be obtained by performing a manual or motorized pullback during the measurement of the fractional flow reserve. In the catheterization laboratory the interventional cardiologist or physician places the FFR wire at the distal location within the coronary of interest. During automatic or manual pullback, the FFR value is continuously measured till the FFR wire reaches the coronary ostium (Sonck et a, “Motorized fractional flow reserve pullback: Accuracy and reproducibility”, Catheter Cardiovasc Interv. 2020 Sep. 1; 96(3):E230-E237).

Optionally, to ensure that the fractional flow reserve values along the coronary artery as measured in the catheterization laboratory (e.g., pullback FFR reference values) are aligned to the spatial coordinates of the MPR image, a co-registration can be performed. This can be performed for instance by the method as described previously by step 208 of FIG. 2.

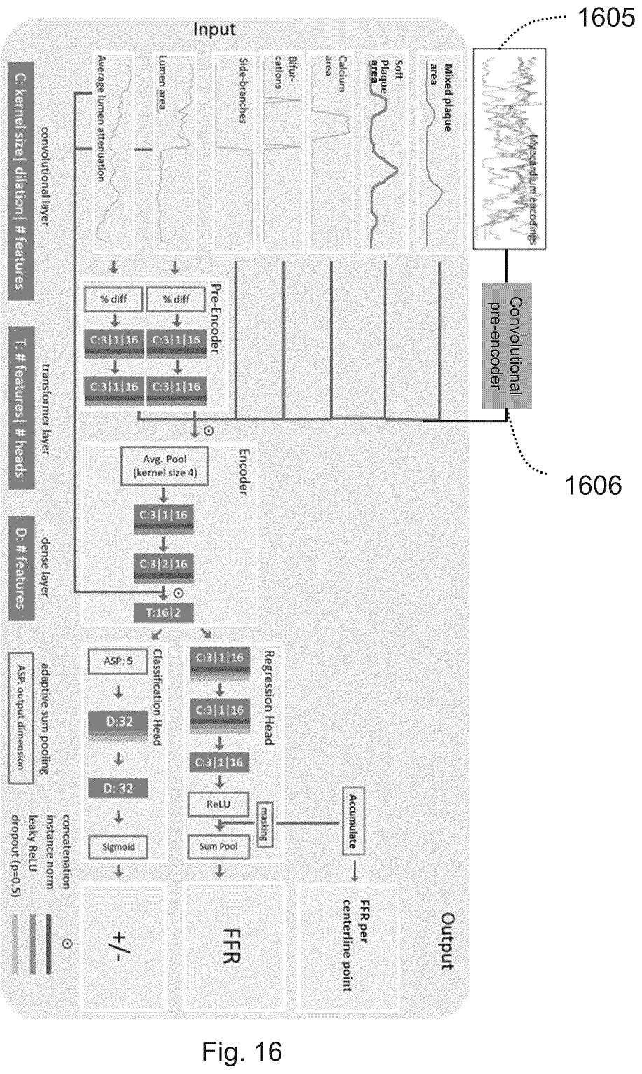

Optionally, in the case x-ray angiographic image data is available for training, the reference FFR per centerline point value can be calculated based on 3D coronary reconstruction using x-ray angiography for instance as taught by Bouwman et al. in U.S. Pat. No. 11,083,377B2 (Method and apparatus for quantitative hemodynamic flow analysis). Bouwman et al describe a method to calculate the vFFR pullback along a coronary of interest based on a three-dimensional coronary reconstruction. Due to the high spatial resolution of X-ray angiography the accuracy of the computed vFFR is considerably high, as described by Masdjedi et al, “Validation of a three-dimensional quantitative coronary angiography-based software to calculate fractional flow reserve: the FAST study”, EuroIntervention. 2020 Sep. 18; 16(7):591-599, in which CAAS Workstation 8.0 (Pie Medical Imaging, the Netherlands) was used to obtain the vFFR value and pullback vFFR values. Within an alternative embodiment the reference standard (209 of FIG. 2, or 1609 of FIG. 16), as an FFR reference value are obtained by calculation of the FFR pullback or calculation of the distal FFR value eliminating the need for invasively measure hemodynamic parameters such as for instance FFR. This can be performed by using the x-ray angiographic image data of the patient and computing the (pullback) FFR value by using for instance the vFFR (vessel-FFR) workflow within CAAS Workstation.

The vFFR method of CAAS Workstation generates a 3D coronary reconstruction using two angiographic x-ray projections at least 30 degrees apart. vFFR is calculated instantaneously by utilizing a proprietary algorithm which incorporates the morphology of the 3D coronary reconstruction and routinely measured patient specific aortic pressure. FIG. 12 shows an example of obtaining a computed FFR pullback of the coronary circumflex by using CAAS Workstation. 1201 shows the segmentation of the coronary circumflex in each 2D X-ray angiographic image, resulting in a 3D reconstruction of the coronary artery (1202). The graph 1203 shows the computed vFFR value along the length of the 3D reconstructed coronary artery. The same approach could also be performed on the CCTA dataset, eliminating the need for corresponding x-ray angiographic image data for each patient.

### Extension 2: FFR Value Per Centerline Point & Additional Artery Characteristics.

This section extends extension 1 with another extension to the method of workflow as described by FIG. 2 which can be implemented by module 304 of FIG. 3. In particular, the architecture of the stenosis assessment network (stenosis assessment network of FIG. 9) employs five different artery characteristics as input. Three artery characteristics (lumen area, lumen attenuation and calcium area) of the five are derived from the MPR image of the vessel of interest (in step 203), and two artery characteristics (side branches, bifurcations) of the five are derived from the extracted coronary centerline tree (step 205). The method described by FIG. 2 is not limited to the described vessel characteristics and can be easily extended to additional vessel characteristics either derived from analysis of the MPR image, analysis of the image data of the vessel of interest or analysis of the coronary centerline tree, but also other characteristics which describe the vessel of interest. For example, data representing the soft plaque area and mixed plaque area of the vessel of interest, which can be derived using the method described by Isgum et al. in U.S. Pat. No. 10,699,407B2 (Method and system for assessing vessel obstruction based on machine learning), can be used as data (signals) that characterize features of the vessel of interest for input to the stenosis assessment network. In summary, Isgum et al. (U.S. Pat. No. 10,699,407B2) first extracted within a CCTA image the centerlines of the coronary arteries. These were used to reconstruct stretched multiplanar reformatted images for the coronary arteries. To perform the automatic analysis, a multi-task recurrent convolutional neural network was applied to coronary artery multiplanar reformatted images to perform two simultaneous multi-class classification tasks. In the first task, the network detects and characterizes the type of coronary artery plaque (no plaque, non-calcified, mixed, calcified). In the second task, the network detects and determines the anatomical significance of the coronary artery stenosis (no stenosis, nonsignificant i.e., <50% luminal narrowing, significant i.e., ≥50% luminal narrowing) and/or the functionally coronary lesion severity.

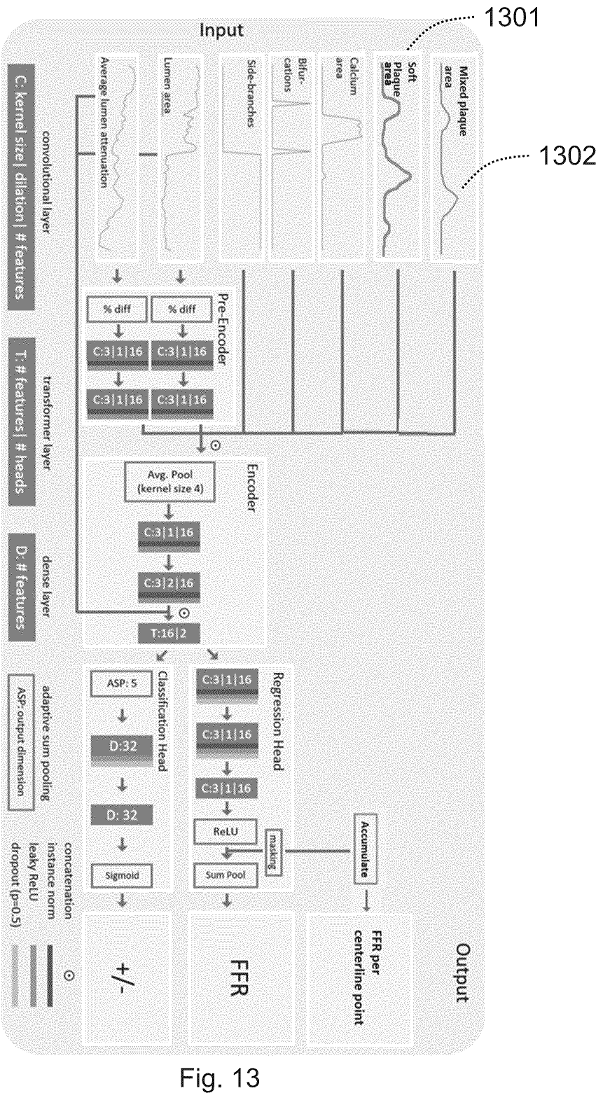

FIG. 13 provides an example that integrates data describing the soft plaque area and the mixed plaque area of a vessel of interest into the architecture of deep learning networks of FIG. 10. Within FIG. 13, the soft plaque area (1301) and the mixed plaque area (1302) are added as characteristics of the vessel of interest. Optionally, the data describing the soft plaque area and mixed plaque area of the vessel of interest can be pre-encoded before passing through the encoder. Furthermore, the data describing one or more features of the vessel of interest can be a binary signal along the MPR image of the vessel of interest, meaning one when soft or mixed plaque is present and zero when not.

Additionally or alternatively, other data (signals) describing characteristics of the vessel of interest can be integrated into the architecture of the deep learning networks of FIG. 2. For example, data (signal) related to the image quality of the MPR view of the vessel of interest can be integrated into the architecture of the deep learning network. For instance, such data (signal) can indicate the presence of stair-step artifact, and/or represent the noise level within the MPR view. In another example, data (signal) related to relative lumen attenuation of the vessel of interest (as compared to another major coronary vessel) can be integrated into the architecture of the deep learning network. For instance, in the case that the vessel of interest represents the RCA, the data (signal) can represent the relative attenuation of the RCA compared to the LAD and/or compared to the LCX. This signal would relate to the flow differences between the main coronary arteries, which is expected to be present in case of functionally significant flow reduction due to an epicardial obstruction and/or (local) microvascular disease.

### Extension 3: Include Myocardium Characteristics

Isgum et al. in U.S. Pat. No. 10,176,575B2 (Method and system for assessing vessel obstruction based on machine learning) recognized that a CCTA image acquisition is typically started once a pre-defined threshold attenuation value has been reached in a pre-defined anatomical structure (most often this concerns the descending aorta), or by waiting a certain delay time after enhancement is first visible in the ascending aorta. This has the effect that the injected contrast medium, once it is present in the coronary arteries, will also be delivered to successively smaller generations of coronary arterioles from where it traverses into the coronary microvasculature, which will lead to enhancement of the myocardium. As functionally significant coronary artery stenosis causes ischemia in the ventricular myocardium, due to the above-described acquisition properties of CCTA, there is a difference in myocardial texture characteristics between normal and ischemic parts of the myocardium at the time of CCTA image acquisition. Isgum et al. describes a method to detect the presence of functional significant stenosis in one or more coronary arteries based on machine learning using features of the myocardium only. In summary, Isgum et al. (U.S. Pat. No. 10,176,575B2) first segmented the myocardium of the CCTA image. Then, from the segmented myocardium, encodings are extracted in an unsupervised manner using a convolutional auto-encoder and used to compute features ([f₁, f₂, f₃, . . . , fₙ]). The convolutional auto-encoder contains two parts, an encoder, and a decoder. The encoder compresses the data to a lower dimensional representation by convolutional and max-pooling layers. The decoder expands the compressed form to reconstruct the input data by deconvolutional and upsampling layers. To represent the entire myocardium, statistics over encodings of all voxels within the myocardium are used as features. Finally, based on the extracted features, patients are classified with a support vector machine to those with or without functionally significant coronary artery stenosis.

This section describes another extension to the method as described by the flowchart of FIG. 2 or FIG. 17 which can be implemented by module 304 of FIG. 3. In particular, the architecture of the stenosis assessment network as described by block 206 of FIG. 2 or block 1706 of FIG. 17, can be adapted to employ data that characterizes the myocardium of the heart.

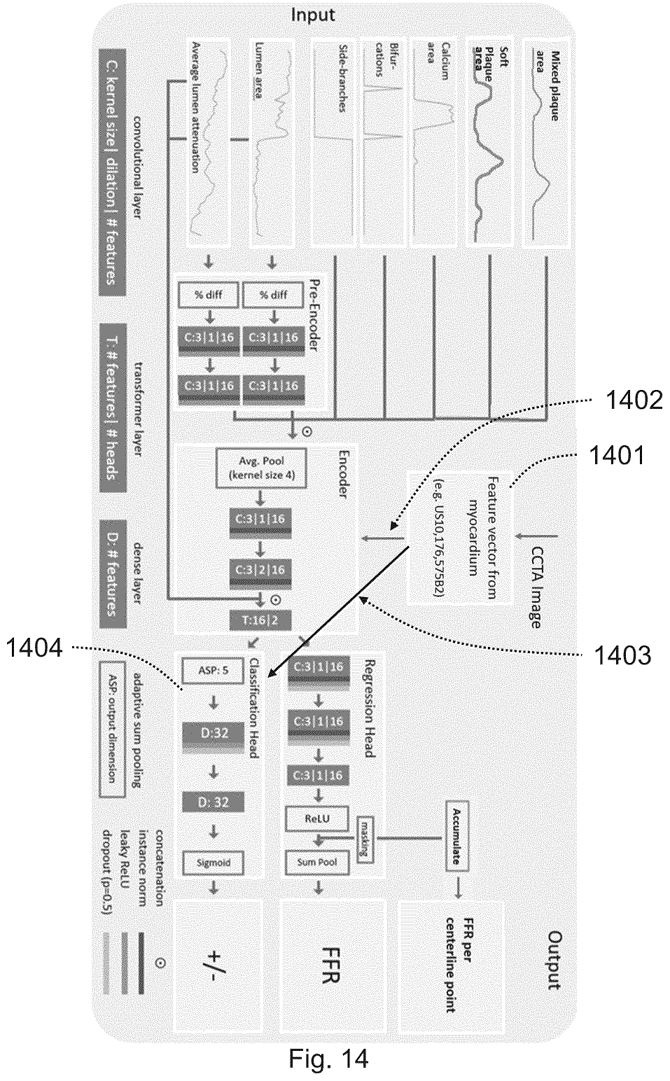

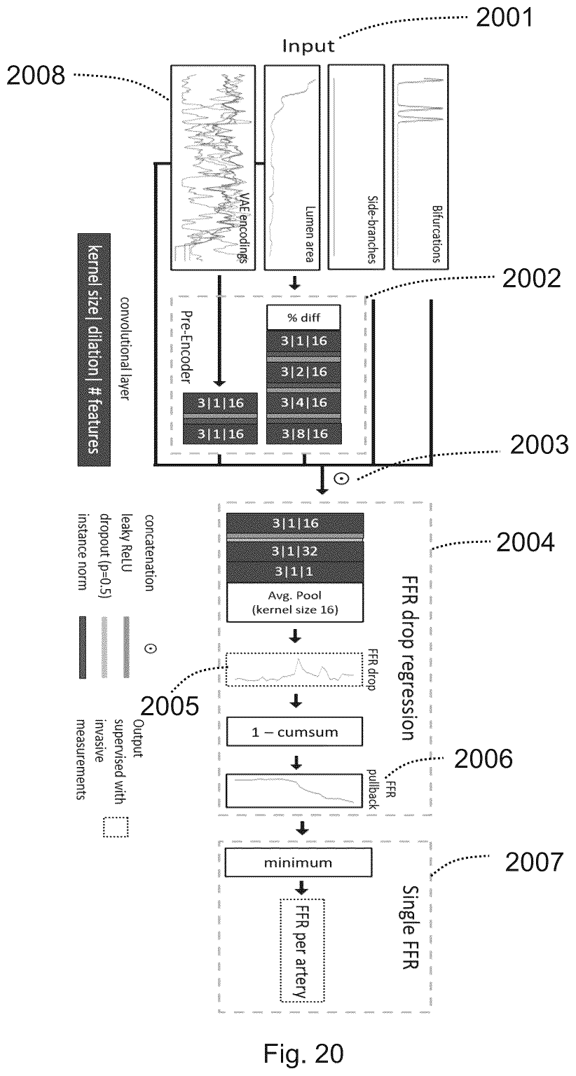

In FIG. 14, an example is provided of integration on characteristics of the myocardium of the heart which are extracted from CCTA image data by, for example, using the methods as described by U.S. Pat. No. 10,176,575B2. Data representing such characteristics of the myocardium is used for input to the encoder of the stenosis assessment network (206, or 1706). For example, a feature vector as obtained from the myocardium analysis as described by U.S. Pat. No. 10,176,575B2 (1401) can be provided as input to the encoder (904 of FIG. 9, or 2003 of FIG. 20). This can be performed by treating the feature vector as an additional input to the encoder (1402), or concatenating the feature vector with the results of the encoder just before one of the dense layers of the classification head (1403) or concatenating the myocardium feature vector resulting from a convolution encoder with other input data (1403).

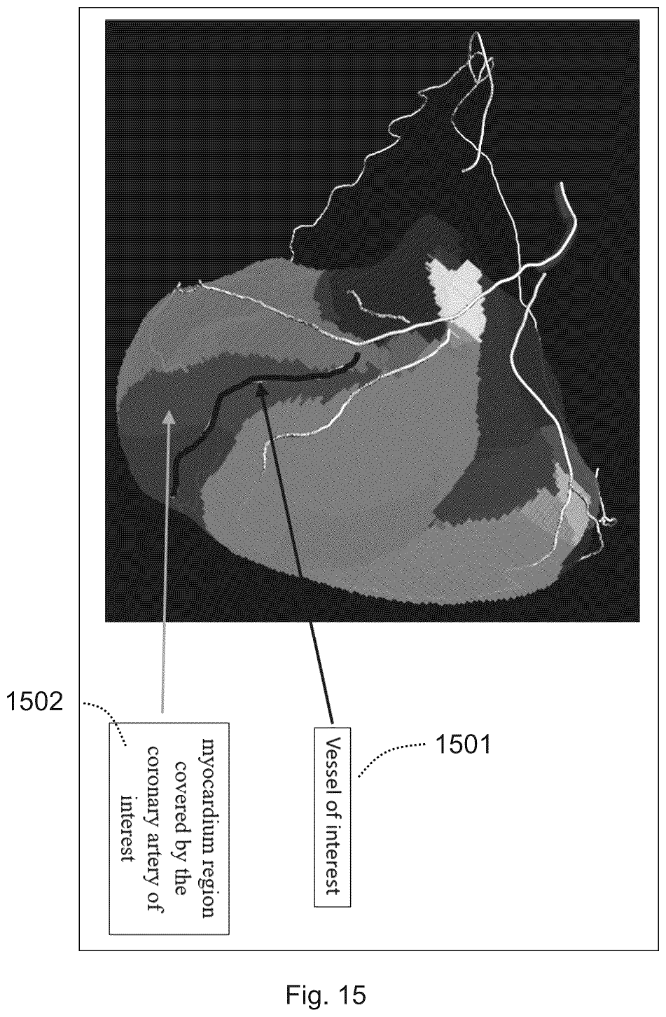

Note that the artery characteristics as described by Extension 2 above can also be used as ‘Additional Information’ (block 155 from FIG. 15 of U.S. Pat. No. 10,176,575B2) within the method as described by U.S. Pat. No. 10,176,575B2.

Alternatively, instead of integrating the feature vector from the myocardium analysis, the FFR classification results as described by U.S. Pat. No. 10,176,575B2 can be integrated into the stenosis assessment network of FIG. 14. In this case, the FFR myocardium classification (e.g., the result of block 158 from FIG. 15 of U.S. Pat. No. 10,176,575B2) can be directly concatenated to ‘Classification Head’ (1404) in FIG. 14. The myocardium feature vector as described by U.S. Pat. No. 10,176,575B2 are based on the full myocardium (e.g., block 156 from FIG. 15 of U.S. Pat. No. 10,176,575B2). In order to concentrate the myocardium characteristics to the region covered by the vessel of interest, the method as described by U.S. Pat. No. 10,176,575B2 can be adjusted. Two possible approaches are described below.

### Approach 1:

The myocardium region covered by the vessel of interest can be defined by applying a Voronoi algorithm (Guibas L et al, “Primitives for the manipulation of general subdivisions and the computations of Voronoi diagrams”, ACM Trans Graph 4:74-123 1985) on the extracted axial trajectory of the vessel of interest for instance using the method of the flowchart of FIG. 28. FIG. 15 provides an example of the subdivision of the myocardium of a heart into regions covered by the coronary arteries. In this example, the vessel of interest is identified by the black artery (1501, second LAD diagonal identified by the black arrow), and the myocardium region that covers this artery is identified by the pink region (1502, identified by the grey arrow). In the case that the vessel of interest covers more than one myocardium region as identified by the Voronoi algorithm, those multiple regions can be merged into one region.

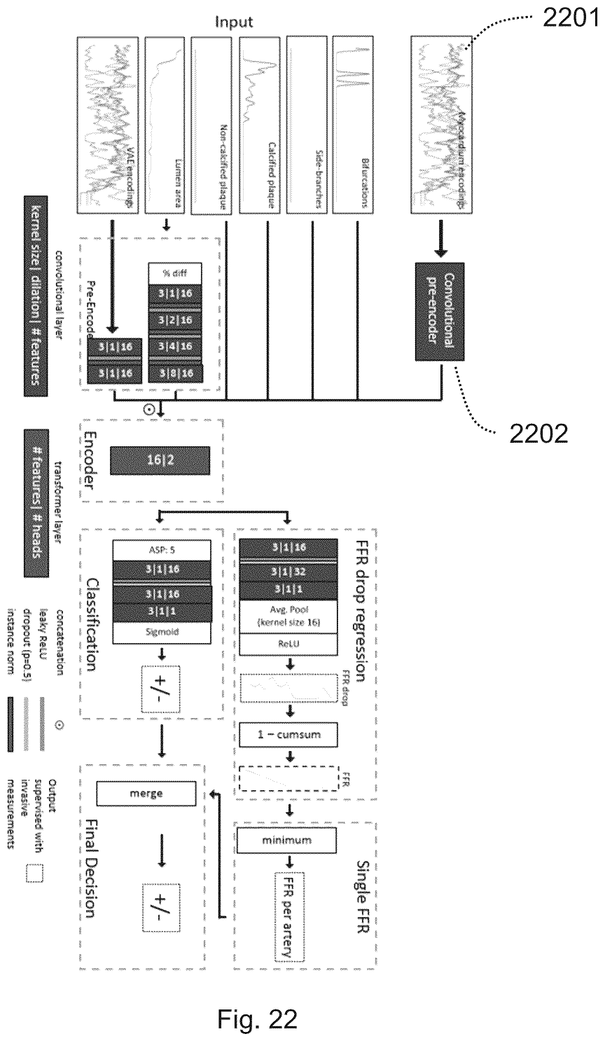

In this approach, the computation of the feature vector as described by U.S. Pat. No. 10,176,575B2 is limited to the defined region and the resulting myocardium feature vector is integrated into the stenosis assessment network (206) as for instance illustrated by FIG. 16. Within FIG. 16, the myocardium feature vector is considered an additional input signal and after pre-encoded using a convolutional network (1606), this additional encoding is fed to the encoder. To compute the myocardium features, for each centerline point along the vessel of interest, (1605, or 2201 of FIG. 22) features are aggregated from a corresponding myocardium territory (1602, or 2202 of FIG. 22). For instance, by interpreting the corresponding myocardium territory as a single cluster and using the standard deviation across the features from all voxels. Alternatively, by interpreting the corresponding myocardium territory as multiple clusters and use the standard deviation to generate a feature vector per cluster and subsequently the maximum across the clusters to create one feature vector for the corresponding myocardium territory. Another alternative is to define the corresponding myocardium territory by the part of the myocardium that is perfused by all downstream segments to a particular centerline point and repeat this for all centerline points within the vessel of interest.

### Approach 2:

In this second approach, the feature vector computation as described by U.S. Pat. No. 10,176,575B2 can be performed within a small region at or around each centerline location along the vessel of interest, and the resulting feature vector can be used as an additional artery characteristic that is input to the encoder of the stenosis assessment network. Alternatively, the feature vector computation as described with reference to FIG. 15 of U.S. Pat. No. 10,699,407B2 can be performed, and the resulting feature vector can be used as an additional artery characteristic (1605, or 2201 of FIG. 22) that is input to the encoder of the stenosis assessment network as illustrated by FIG. 16. Optionally, a pre-encoder (1606, or 2202 of FIG. 22) can be applied to such new artery characteristic(s).

Myocardial ischemia occurs when blood flow to the heart muscle (myocardium) is obstructed by a partial or complete blockage of a coronary artery by a buildup of plaques (atherosclerosis). This typically results in chest pain (angina) experienced by the patient. Up to half of the patients undergoing elective coronary angiography for the investigation of chest pain do not present with evidence of obstructive coronary artery disease. These patients are often discharged with a diagnosis of non-cardiac chest pain, yet many could have an ischemic basis for their symptoms. This type of ischemic chest pain in the absence of obstructive coronary artery disease is referred to as INOCA (ischemia with non-obstructive coronary arteries). INOCA involves a supply-demand mismatch of myocardial oxygen caused by microvascular disfunction. Microvascular disfunction involves dysfunction of the small vessels that supply the myocardium and is more common in woman, especially during middle age. INOCA can also be caused by vasospastic disorder which is caused by spasm of coronary arteries.

The strong point of the method described by Isgum et al. in U.S. Pat. No. 10,176,575B2 (Method and system for assessing vessel obstruction based on machine learning), is that both obstructive coronary ischemia as well as non-obstructive coronary artery ischemia can be identified. However, from a patient treatment point of view it is required to identify the differences, since the treatment strategy is different between obstructive coronary ischemia and non-obstructive coronary ischemia.

With the integration of the method as described by U.S. Pat. No. 10,176,575B2, identification of microvascular disfunction can be integrated into the stenosis assessment network.

As INOCA is microvascular disfunction without epicardial coronary artery obstruction, this can be identified by examination of the vessel of interest in the case that the output of deep learning networks (e.g., the network provided by FIG. 9, 10, 13, 14, 16, 20, 21 or 22) identifies functionally significant obstruction of blood flow. This can be done by:
examination of the artery characteristics resulting from the ‘Artery Characterization’ network (FIG. 6 or FIG. 8 or FIGS. 19a and 19b). For instance, performing a QCA analysis on the lumen area graph. If for instance an obstruction severity of >50% then this is classified as obstructive coronary artery disease.
performing a QCA analysis on the segmentation of the coronary tree for instance by the method as described by Wolterink et al. “Graph convolutional networks for coronary artery segmentation in cardiac CT angiography”, in International Workshop on Graph Learning in Medical Imaging. Springer, Cham, 2019. In which the QCA analysis is performed by for instance the methods as described by Hof et al. in WO2012/028190A1 (Method and apparatus for quantitative analysis of a tree of recursively splitting tubular organs). If for instance an obstruction severity of >50% in the coronary tree is identified, then this is classified as obstructive coronary artery disease.
combining of the QCA analysis as described above with the amount of coronary plaque, either as obtained by extension 2 or by the method as described by Isgum et al. in U.S. Pat. No. 10,699,407B2 or as taught by Wolterink et al. “Automatic Coronary Artery Calcium Scoring in Cardiac CT Angiography Using Paired Convolutional Neural Networks”, Medical Image Analysis, 2016 or as for instance taught by Dey et al, “Automated 3-dimensional quantification of noncalcified and calcified coronary plaque from coronary CT angiography”, Cardiovasc Comput Tomogr. 2009, 3(6):372-382.
incorporating an additional classifier in the network architecture of FIG. 9, 10, 13, 14, 16, 20, 21 or 22. This can involve adding another ‘Classification INOCA Head’ similar as the ‘Classification Head’ which is trained to output a value characterizing microvascular dysfunction, like for instance IMR (index of microcirculatory resistance) and/or coronary flow reserve.

Optionally, the method as described by U.S. Pat. No. 10,176,575B2 (and integrated into the deep learning networks as described herein) can be improved by including a CT calcium scan. In contrast to a CCTA scan, a CT calcium scan is acquired without injection of any contrast medium. Incorporating the CT calcium scan provides information of the myocardium without any presence of contrast liquid, resulting in a ‘baseline myocardium’. By incorporating the CT calcium scan, the machine learning network is able to integrate myocardium intensities without any contrast enhancement, and thereby improving the detection of subtle contrast changes between the healthy myocardium regions and ischemic myocardium regions. After registration of the CT calcium scan to the CCTA scan, both image datasets can be used in the method as described by U.S. Pat. No. 10,176,575B2.

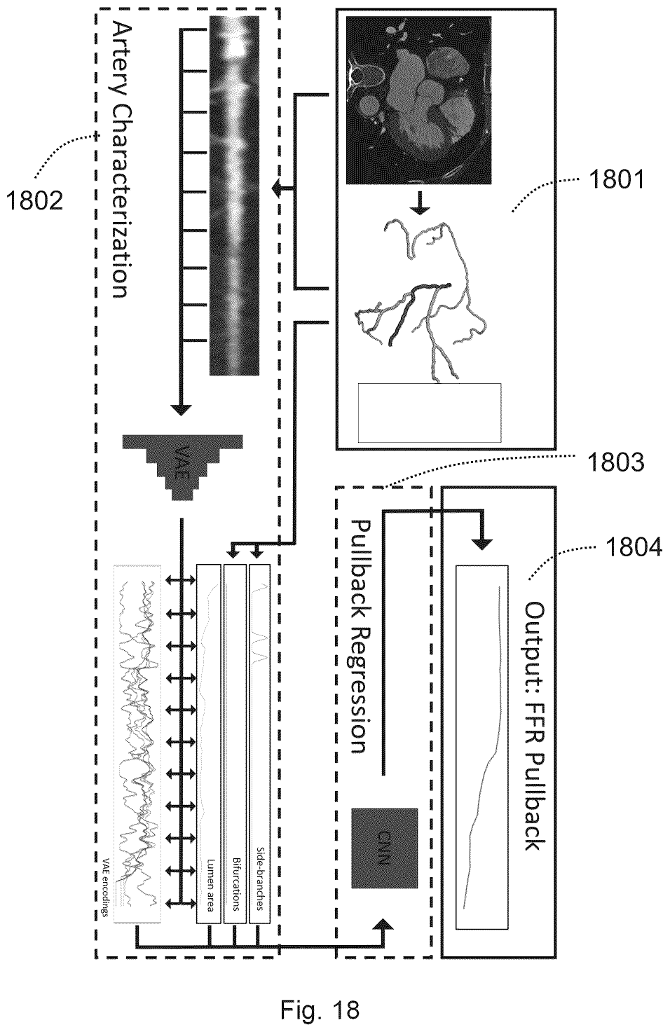

FIG. 17 describes another embodiment of the present application. The therein-depicted steps can, obviously, be performed in any logical sequence and can be omitted in parts. FIG. 17 illustrates a flowchart of a machine learning based method for determining functionally significant lesion severity along the axial trajectory of the vessel of interest. The machine learning based method is similar to the one described with reference to FIG. 2 and extension 2 ‘FFR value per centerline point & additional artery characteristics’, although there are some fundamental differences. To enable fast, quantitative assessment of the distribution of CAD, a deep learning-based method is used for prediction of the FFR pullback given the MPR image of the vessel of interest as input. The method consists of two stages and is illustrated at a high-level by FIG. 18. First from the input CCTA image, the axial trajectory (e.g., centerline) of the vessel of interest is extracted (1801) and an MPR image is generated from the axial trajectory of the vessel of interest and the CCTA image data of the vessel of interest. In the first stage of the deep learning-based method, the vessel of interest is characterized along the length of the MPR image (1802). In embodiments, this first stage can combine unsupervised learning and supervised learning. The unsupervised learning provides for extraction of non-hand-crafted features from the MPR image. The supervised learning explicitly incorporates clinical knowledge into the machine learning model. Subsequently, features that characterize the vessel of interest derived from both the supervised and unsupervised learning methods are fed to a deep learning network (1803) to predict the FFR drop along the artery in the second stage (1804). To enable the distinction between focal and diffuse CAD, it is important that the prediction of the FFR drop along the axial trajectory of the vessel of interest is accurate. This is archived by supervising the FFR pullback prediction with the FFR pullback reference (1709, FIG. 17). For this, a novel loss function is used which is inspired by the Earth Mover's Distance (EMD). As registration of the reference FFR signal with an MPR image is challenging, a mismatch cannot be excluded. Hence, straightforwardly using a loss function like the mean absolute error (MAE) that evaluates all vessel points separately may lead to an ambiguous loss signal if the overlap between the reference FFR drops and the true FFR drop in the input artery is low. Instead, the proposed loss function increases continuously with the distance between the predicted FFR drop and the reference FFR drop. While the EMD loss concentrates the FFR drops around the assumed lesion locations, uncertainty of correct lesion location may lead to a tendency to underestimate the sharpness of FFR drops. As distinguishing between focal and diffuse CAD requires correct prediction of the steepness of the FFR pullback, an additional loss function is designed to penalize the histogram of FFR drops. The deep learning-based method for prediction of the FFR pullback will be described in detail with reference to FIG. 17.

At the first step 1701, a CCTA image dataset is obtained of the vessel of interest. Such CCTA image dataset represents a volumetric CCTA image dataset, for instance a single contrast enhanced CCTA dataset and is identical to the description of step 201 of FIG. 2.

In step 1702, an axial trajectory extending along the vessel of interest is extracted and this step is identical to the description of step 202 of FIG. 2.

In step 1703, a three-dimensional (3D) multi-planar reformatted (MPR) image is created of the vessel of interest and this step is identical to the description of step 203 of FIG. 2.

In steps 1704 and 1705, the first stage of the deep learning-based method is employed. In this first stage, features of the vessel of interest can be extracted through a combination of unsupervised learning and supervised learning.

In embodiments, this first stage employs an artery characterization network (e.g., 1704 of FIG. 17, and 1802 of FIG. 18) that employs unsupervised machine learning to characterize features of the vessel of interest given the MPR image of step 1703 as input.

In embodiments, the artery characterization network (e.g., 1704 of FIG. 17, and 1802 of FIG. 18) can employ a variational autoencoder (VAE) which can be configured to extract non-hand-crafted features from the MPR image. Variational autoencoder (VAE) are generative models, which approximate data generating distributions as described by Kingma et al, “Auto-encoding variational bayes”, arXiv arXiv:1312.6114, 2013. Through approximation and compression, the resulting models capture the underlying data manifold; a constrained, smooth, continuous, lower dimensional latent (feature) space where data is distributed (Kingma et al, “Semi-supervised learning with deep generative models”, Advances in neural information processing systems, 2014, pp. 3581-3589). A VAE enforces latent features with independent normal distributions, increasing the interpretability and denseness of the latent space with respect to a conventional convolutional autoencoder. These advantageous properties of the latent space are used within the machine learning based Artery Characterization Network as described by this step.

A typical VAE includes two major parts, an encoder and a decoder. The encoder compresses (encodes) the data to lower dimensional latent space by convolutional operations and down-sampling (max-pooling), and subsequently expands (decodes) the compressed form to reconstruct the input data by deconvolutional operations and upsampling (unpooling). Training the VAE, while minimizing a distance loss between the encoder input and the decoder output, ensures that the abstract encodings, generated from the input, contain sufficient information to reconstruct it with low error. Once the VAE is trained, the decoder is removed, and the encoder is used to generate encodings for unseen data.

FIGS. 19a and 19b show an example of the architecture of a VAE network that characterizes features of a vessel of interest given an MPR image (from step 1703) as input. The VAE network includes a convolutional encoder (1902) configured to calculate a number of features (z) from a stack of a predefined amount of successive cross-sectional slices, for example thirteen cross-sectional slices of the input MPR image (1901). The convolutional encoder (1902) can include convolutional blocks, containing for example two convolutional layers, batch normalization and the ReLU activation. The second convolutional layer in each block can have a stride of, for example 2, which enables down sampling the resolution. To increase the amount of context information, the output of the network after the second and the fourth convolutional block can be concatenated (“fused”) with the output of the same network applied to for example the 6 and 3 adjacent MPR slices, respectively, which increases the receptive field in z direction. Furthermore, bifurcation information (1903) for the vessel of interest can be injected into the convolution encoder, for example, before the first, the third and after the last convolutional block. In embodiments, the bifurcation information can be a bifurcation feature map indicating whether the MPR slices are located at a bifurcation, and the bifurcation feature map can be injected into the respective convolution blocks by concatenation. The bifurcation information can be generated or derived and supplied by block 1705 of FIG. 17 using the methods as described with respect to step 204 of FIG. 2.

To configure the encoder (1902) to extract relevant information from an arbitrary input MPR image (1901) into encodings that represent a distribution of latent features in the input MPR image, the encoder (1902) and a primary decoder (1904) are trained to reconstruct the central input slice of the input stack from the encodings (z) output by the encoder (1902). Like the encoder (1902), the primary decoder (1904) consists of alternating convolutional blocks and pooling operations. However, for the primary decoder (1904), the last convolutional layer in each block is transposed to enable up-sampling. Furthermore, only a single slice is reconstructed instead of the whole input stack.

To enhance the machine learning network to extract features that characterize artery and plaque geometry, auxiliary decoders (1905a, 1905b) are configured to process the encodings (or part thereof) output by the encoder (1902) to predict segmentation masks for the lumen characteristics and the calcified and the non-calcified plaque characteristics. The auxiliary decoder 1905a can be embodied by a linear layer that processes the encodings (z) output by the encoder (1902) by regression to output feature data that characterizes lumen area of the vessel of interest over the axial trajectory of the vessel of interest (1906). The auxiliary decoder 1905b can have an architecture similar to the primary decoder (1904), with a difference in the last layer, which has four channels and uses the softmax activation to output feature data that characterizes lumen attenuation as well as calcified plaque and non-calcified plaque of the vessel of interest over the axial trajectory of the vessel of interest. Optionally, the auxiliary decoders (1905a, 1905b) can embody multiple linear layers that are configured to extract feature data that characterize artery and plaque geometry from the encodings (z) output by the encoder (1902).

Moreover, the encoder (1902) can be configured with a predefined amount of, for instance 32, predictor means and standard deviations (1907) that generate the encodings (z) output by the encoder (1902). The encodings (z) output by predictor means and standard deviations (1907), or certain subsets thereof, can be supplied as input to auxiliary decoders (1905a, 1905b) to enable the auxiliary decoders (1905a, 1905b) to extract the relevant feature data from the encodings (z). The encodings (z) output by the predictor means and stand deviations (1907) and the regressed lumen area (1906) from the feature data output by the auxiliary decoders (1905a) can be supplied to the machine learning based FFR pullback network (1706 of FIG. 17, 2008 of FIG. 20). Optionally, the feature data output by the auxiliary decoders (1905b) can also be supplied to the machine learning based FFR pullback network (1706 of FIG. 17, 2008 of FIG. 20).

The VAE of FIGS. 19a and 19b provides a machine learning based artery characterization network (1704 of FIG. 17) that combines supervised and unsupervised characteristics extracted from the MPR image and from the coronary tree. FIG. 19b further illustrates the unsupervised and supervised parts of the VAE. Both FIGS. 19a and 19b show the VAE architecture of the machine learning based artery characterization network. Stacks of a predefined number of slices around a central MPR slice at a certain position along the centerline of the vessel of interest are encoded using a convolutional encoder (1902). The encoder (1902) consists of, for example, 5 convolutional blocks, each comprising 2 convolutions. Bifurcation information (1903) can be injected into the convolution blocks of the encoder (1902) as described above. At the output of the encoder (1902), the latent vector encodings (z) are sampled from the predicted feature vectors μ (mean) and σ (standard deviation). The auxiliary decoder 1905a receives the latent vector encodings (z) as input and directly regresses the lumen area of the vessel of interest over the axial trajectory of the vessel of interest. The auxiliary decoder 1905b receives only a subset of the latent vector encodings (z) as input and reconstructs the segmentations for lumen attenuation as well as calcified plaque and non-calcified plaque characteristics of the vessel of interest over the axial trajectory of the vessel of interest. The primary decoder (1904) and the auxiliary decoder (1905b) are disconnected during handling of unseen data.

To train the supervised part of the VAE (1951 of FIG. 19b), reference values (1708 of FIG. 17) can be used. The reference values can be obtained from a database which contains data of multiple patients. Each set within the database contains for each patient a) contrast enhanced CT image datasets (1701 represents reference image sets during the training phase) and corresponding b) artery characteristics reference values. For the artery characteristics, reference annotations of the coronary artery lumen, and plaque (calcified, non-calcified). These reference annotations can be created by manual segmentation of CCTA datasets used during the training, or by automatic segmentations of the lumen and plaque components of CCTA datasets used during the training followed by manual corrections when needed. Automatic segmentation of the lumen and plaque components can for instance be performed by segmented in the original CT image volumes using the approach as described by Wolterink et al, “Automatic coronary artery calcium scoring in cardiac CT angiography using paired convolutional neural networks”, Medical Image Analysis 34, 123-136 (2016) and Wolterink et al, “Graph Convolutional Networks for Coronary Artery Segmentation in Cardiac CT Angiography”. In: Zhang D, Zhou L, Jie B, Liu M, editors. Graph Learning in Medical Imaging. Lecture Notes in Computer Science. Cham: Springer International Publishing (2019). p. 62-9. Thereafter, automatic segmentations are transferred to the MPR image of the artery, visually inspected and corrected when needed. As X-ray angiography is superior in image resolution as compared to CCTA, within a preferred embodiment the lumen segmentation on MPR image is guided by QCA3D analysis results extracted from X-ray angiographic image data from the same patient which is acquired within three months of the acquisition of the CCTA data of the patient in question. The QCA3D can be performed for instance by CAAS Workstation (Pie Medical Imaging BV, the Netherlands), using the approach as described by Girasis C, et al, “Advanced three-dimensional quantitative coronary angiographic assessment of bifurcation lesions: methodology and phantom validation”, EuroIntervention 2013; 8: 1451-1460.

To ensure that the reference values 1708 are aligned to the spatial coordinates of the MPR image (1703), the reference values can be transformed to the MPR image domain. In case the reference values (e.g., annotation of lumen and plaque type) are obtained by using the MPR image as a result from step 1703, this step may be skipped. When the reference values are obtained, for instance by annotation using the contrast enhanced CT datasets (step 1701), this step transforms the annotation into the MPR view. Such a transformation is performed by using the extracted vessel trajectory as a result of step 1702.

In embodiments, during training of the machine learning based artery characterization network (1704 of FIG. 17, and 1802 of FIG. 18), three separate loss functions can be used. A mean absolute error (MAE) loss function can be used to minimize the loss in the MPR reconstruction loss (1904). Likewise, a MAE loss function can be used to supervise regression of lumen features (1906). For the segmentation loss (1905), a binary cross-entropy loss function can be used.

In embodiments, the latent space encodings (z) of the VAE can be regularized using the Kullback-Leibler divergence to facilitate a dense latent space and disentangled latent features. The Kullback-Leibler divergence metric is a statistical measurement from information theory that is commonly used to quantify the difference between one probability distribution from a reference probability distribution. It is a non-symmetric metric that measures the relative entropy or difference in information represented by two distributions. It can be thought of as measuring the distance between two data distributions showing how different the two distributions are from each other. The Kullback-Leibler divergence penalizes the differences between the normal distribution parameterized by the predicted means and standard deviations, and the standard normal distribution.

In the VAE, the output (1907) of the encoder can generate the latent space encodings (z) by predicting a mean and a standard deviation for each feature and subsequently drawing a random sample from a normal distribution parameterized by this mean and standard deviation. Without any additional loss, a network with this resampling would always predict a standard deviation of 0, as this minimizes the randomness of predictions and hence maximizes the transmitted information. However, similar to a regular convolutional autoencoder, this would lead to a latent space with gaps (regions with output that does not look like a realistic coronary artery) and entangled (correlated and meaningless) latent features. Instead, to facilitate a dense latent space and disentangled latent features, the latent space of the VAE can be regularized using the Kullback-Leibler divergence (KLD, a measure for differences between distributions) between the normal distributions parameterized by the predicted means and standard deviations, and standard normal distributions. Therefore, the network has to decide how much precision is required for each latent, balancing the reconstruction loss and the regularization. This has the following advantages over a regular autoencoder:
Intuitively, the process of sampling in combination with the KLD encourages denseness of the latent space by enforcing all points close to the predicted means to yield approximately correct output as well.
By sampling the latent space independently, the features are encouraged to be uncorrelated, which tends to yield features that are more meaningful and hence likely more suitable for our stenosis assessment.

Optionally, the auxiliary output decoder (1905b) of the VAE can be kept connected during handling of unseen data and the predicted lumen, calcified plaque and non-calcified place can be used to calculate geometric parameters from the vessel of interest by using quantitative coronary analysis (QCA). First from the segmentation regions (result of the auxiliary output decoder (1905) of the VAE), such as vessel lumen, vessel plaque (calcified, and non-calcified), a 3D model is created either in the spatial coordinate system of the MPR image (1703) or in the spatial coordinate system of the CT image (1701). Next, from the 3D model, anatomical results are computed for instance using the approach as described by Girasis C, et al, “Advanced three-dimensional quantitative coronary angiographic assessment of bifurcation lesions: methodology and phantom validation”, EuroIntervention 2013; 8: 1451-1460. Examples of such quantitative anatomical results are, length, equivalent diameter along the axial trajectory of the vessel of interest, cross section area along the axial trajectory of the vessel of interest, obstruction length, minimum equivalent diameter, minimum luminal area, percentage diameter stenosis, percentage area stenosis, reference diameter/area, vessel volume, plaque (calcified, non-calcified) volume, plaque burden (plaque volume/vessel volume). The healthy reference diameter or area graph, representing the diameter/area in case the vessel is healthy, is computed by for example fitting a line through the diameter or area values along the axial trajectory of the vessel of interest in which the diameter or area values within the lesion extent are excluded during the fitting as described by Gronenschild E, et al. in “CAAS II: A Second Generation system for Off-Line and On-Line Quantitative Coronary Angiography”, Cardiovascular Diagnosis 1994; 33: 61-75.

Alternatively, vessel characteristics such as lumen attenuation, lumen area, calcium area, soft plaque area, and mixed plaque area along the axial trajectory of the vessel of interest can be extracted as described by the method at step 204 of FIG. 2 and further explained with reference to FIG. 8.

In embodiments, this first stage of the deep learning-based method (1704) can also employ supervised machine learning to characterize features of the vessel of interest given the MPR image of step 1703 as input. To account for the impact of the artery's geometry and local appearance on blood flow, additional characteristics can be defined. Specifically, in step 1705 of FIG. 17, other data (signals) that characterize features of the vessel of interest along the axial trajectory (e.g., centerline) of the vessel of interest (as obtained from step 1702 of FIG. 17) can be extracted from CCTA image dataset of step 1701. For example, for each point along the axial trajectory of the vessel of interest, two or more additional characteristics can be extracted. One of the additional characteristics can indicate the presence of bifurcations at the artery centerline point. Another additional characteristic can indicate whether a centerline point belongs to a main branch (i.e., left main (LM), LAD, LCX, RCA) or a side-branch. Other examples of additional characteristics that characterize the vessel of interest could be the presence of a stent and/or type of stent (from a previous coronary intervention procedure) at the vessel centerline point.

The second stage of the deep learning-based method (step 1706 of FIG. 17, and 1803 of FIG. 18) employs a machine learning based FFR pullback network that is configured to characterize FFR pullback along the axial trajectory of the vessel of interest given the features of the vessel of interest output from the artery characterization network (1704) and coronary tree characteristics (1705).

In embodiments, the machine learning based FFR pullback network can utilize a CNN network architecture as described herein. FIG. 20 shows an example CNN network architecture for characterizing FFR pullback along the axial trajectory of the vessel of interest. The input signals are represented by 2001. To remove the trend of the lumen area signal, the percentage difference is calculated at each position along the axial trajectory of the vessel of interest with respect to the value at the previous location. The calculated lumen area percentage difference signal and the VAE encodings are separately pre-encoded (2002) to extract independent features from local environments. For the lumen area signal a number of convolutional layers (for example four) with rising levels of dilations to enlarge the receptive field are applied to encompass the entire vessel of interest. The VAE encodings are pre-encoded preferably with a smaller number of convolutions layers (for example two), this to prevent overfitting. Thereafter we concatenate the resulting features with the remaining characteristics (2003) and feed the resulting encodings to a common convolutional pathway for regression of the FFR pullback (2004), which comprises convolutional layers, average pooling and a ReLU activation function. The FFR drop regression network (2004) is inspired by the additive nature of sequential flow resistances. The FFR drop regression network predicts the FFR pullback by first predicting the FFR drop per point in the artery of interest (2005). This way, the prediction target at each point is independent of the previous outputs. Subsequently the FFR pullback (2006) is calculated by taking the cumulative sum over the predicted FFR drops. To obtain an artery level FFR prediction (2007) from the predicted FFR drop, the minimum of the FFR pullback is taken which is identical to the most distal value. The FFR pullback prediction network uses a kernel size of, for example three, for all convolutions with zero-padding to preserve feature size. Furthermore, we utilize for example 16 filter maps to balance expressiveness and overfitting. To account for potential misregistration with the reference FFR drop per point, we make use of average pooling. To further prevent overfitting, we set dropout probabilities for instance to 0.5, and instance normalization is used throughout the FFR drop regression network.

To train the machine learning based FFR pullback network (1706 of FIG. 17, and 1803 of FIG. 18), a reference standard is used (1709 of FIG. 17). The reference standard can be provided from a database which contains data of multiple patients. Each set within the database contains for each patient a) contrast enhanced CT image datasets (1701 represents reference image sets during the training phase) and corresponding b) CAD related reference values representing a hemodynamic index for assessment of functionally significant coronary artery obstruction. For example, the CAD related reference values may represent at least one invasively measured fractional flow reserve, coronary flow reserve, instantaneous wave-free ratio, resting full-cycle ratio, diastolic hyperemia free ratio, diastolic pressure ratio, resting Pd/Pa ratio, hyperemic myocardium perfusion, index of microcirculatory resistance, pressure drop along a coronary artery and the fractional flow reserve along a coronary artery. Note that the reference standard (1709) and the artery reference values (1708) require the same (reference) contrast enhanced CT image datasets.

With the focus of prediction of FFR along the axial trajectory of the vessel of interest, the reference standard needs to represent the FFR along the axial trajectory of the vessel of interest. This can be obtained from a manual or motorized invasive FFR pullback. In the catheterization laboratory the interventional cardiologist or physician places the FFR wire at the distal location within the coronary of interest. During automatic or manual pullback, the FFR value is continuously measured till the FFR wire reaches the coronary ostium (Sonck et a, “Motorized fractional flow reserve pullback: Accuracy and reproducibility”, Catheter Cardiovasc Interv. 2020 Sep. 1; 96(3):E230-E237). Optionally, in the case x-ray angiographic image data is available for training, the reference FFR per centerline point value can be calculated based on 3D coronary reconstruction using x-ray angiography for instance as taught by Bouwman et al. in U.S. Pat. No. 11,083,377B2 (Method and apparatus for quantitative hemodynamic flow analysis) and further described before at the description of extension 1 of current patent application. Bouwman et al describe a method to calculate the vFFR pullback along a coronary of interest based on a three-dimensional coronary reconstruction. In case the CAD related reference values represent a non-hyperemic index (e.g., instantaneous wave-free ratio, resting full-cycle ratio, diastolic hyperemia free ratio, diastolic pressure ratio, resting Pd/Pa ratio), the output of the FFR drop regression also represents such non-hyperemic indices.

In embodiments, during training of the machine learning based FFR pullback network (1706 of FIG. 17, and 1803 of FIG. 18), the prediction of the FFR drop can be supervised with the reference FFR drop along the axial trajectory of the vessel of interest. For this, a novel loss function can be used which is inspired by the Earth Mover's Distance (EMD) and introduced a so-called EMD loss. Instead of locally comparing two probability distributions, EMD is a way to measure the global similarity between the distributions. Intuitively, EMD is the minimum amount of “work” required to transform one distribution into another. Thereby, “work” is defined as the amount of probability mass that needs to be moved, multiplied by the distance it needs to be moved. Therefore, unlike a point-wise comparison like the mean absolute error (MAE), this loss function increases continuously with the distance between the predicted FFR drop and the reference FFR drop. This potentially yields improved gradient updates in case of misregistration between the predicted and the reference pullback curve. In a single dimension, the EMD between two probability distributions p₁ and p₂, discretized via xᵢ for i∈[0, l], is calculated as:

$$EMD = {\sum }_{j = 0}^{i} \vert {\sum }_{i = 0}^{j} ( {p}_{1} ( {x}_{i} ) - {p}_{2} ( {x}_{i} ) ) \vert \qquad ( equation 2 )$$

wherein l represents the length of the vessel of interest and i is the running index.

The formula of equation 2 is used to calculate the loss between the predicted and the reference FFR drop. Intuitively, this calculation corresponds to the accumulated difference between the FFR curves:

$$\begin{matrix}{L}_{FFR} & = & {\sum }_{j = 0}^{i} \vert {\sum }_{i = 0}^{j} Δ {FFR}_{pred} ( {x}_{i} ) - Δ {FFR}_{ref} ( {x}_{i} ) \vert & ( equation 3 ) \\ & = & {\sum }_{j = 0}^{i} \vert {FFR}_{pred} ( {x}_{j} ) - {FFR}_{ref} ( {x}_{j} ) \vert & ( equation 4 ) \\ & & & \end{matrix}$$

However, as calculation of the FFR from the FFR drop is asymmetric, L_{FFR} punishes proximal FFR drop differences more than distal ones. To instead treat differences in FFR drops equally regardless of their location, we design a symmetric version of L_{FFR}. For this, we add a second term L_{FFR} calculated from the hypothetical FFR curve computed by adding up FFR drops from distal to proximal:

$${L}_{FFR}^{s} = {L}_{FFR} + {\overline{L}}_{FFR} \qquad ( equation 5 )$$

$${\overline{L}}_{FFR} = {\sum }_{j = 0}^{i} \vert {\sum }_{i = 0}^{j} Δ {FFR}_{pred} ( {x}_{j - i} ) - Δ {FFR}_{ref} ( {x}_{i - j} ) \vert \qquad ( equation 6 )$$

Optionally, to enable distinguishing focal from diffuse FFR drops, it is crucial that the predicted pullback curve drops with similar sharpness as the reference pullback curve. This is enforced by penalizing differences between the histogram of the predicted FFR drops and the reference; and introduced a so-called histogram loss. However, straightforwardly binning output values is not differentiable and therefore does not enable training of a neural network. To solve this problem, a Parzen-Rosenblatt window approach is used by approximating the bins using normal distributions with means located at the respective bin's center. The Parzen-Rosenblatt window method is a widely used non-parametric approach to estimate a probability density function p(x) for a specific point p(x) from a sample p(xn) that doesn't require any knowledge or assumption about the underlying distribution, as described by Parzen et al, “On Estimation of a Probability Density Function and Mode”, The Annals of Mathematical Statistics 33 (3), 1962, pp. 1065-1076. Specifically, we employ for instance 32 normal distributions with sigma for instance 0.1, equidistantly distributed between for instance −0.1 and 0.5, i.e., the expected range of FFR drops. This results in a series of values describing the number of occurrences of FFR drops with certain magnitude (i.e., histograms) in both the predicted and the reference FFR curve. Thereafter differences are penalized between these histograms using the weighted absolute error. Each bin is weighted according to the magnitude of the corresponding FFR drop, giving more weight to larger FFR drops which are less common but clinically more important.

In embodiments, negative FFR drops can be present within the reference standard (1709). For example, such negative FFR drops can result from invasive pullback pressure measurements. Such negative FFR drops, meaning an increase in FFR from proximal to distal, is mostly related to the hydrostatic pressure effect as described by Harle T, et al, “Influence of hydrostatic pressure on intracoronary indices of stenosis severity in vivo”, Clin Res Cardiol 2018 107:222-232. Optionality, to exclude any hydrostatic pressure another loss function can be included, a so called Monotony loss. By disabling the ReLU activation function as the last layer (see description of Step 1706 of FIG. 17), negative FFR drops are allowed. These are discouraged by penalizing the sum of negative FFR drops. Finally, in step 1707 of FIG. 17 the output (2006, FIG. 20, and 2007 of FIG. 20) is provided. The output is data that represents FFR pullback along the axial trajectory of the vessel of interest.

FIG. 11 shows an exemplary graphical user interface (display screens) that visually conveys the result from step 1507 to a user. In FIG. 11, 1102 shows a volume-rendered image of the CCTA data in which the three main coronary arteries are enhanced; right coronary artery (RCA), left anterior descending coronary artery (LAD), and the left circumflex coronary artery (LCX). The RCA is the vessel of interest, and results in a multiplanar reconstruction (MPR) illustrated by 1101-part B of FIG. 11 in which the left side (1103) corresponds to the ostium (proximal) location of the RCA and right side (1104) corresponds to the distal location of the RCA. 1101-part C shows the lumen area or lumen diameter graph along the RCA, in which the x-axis corresponds to the x-axis of the MPR view of 1101 part B and the centerline of the RCA. Finally, 1101-part D shows the FFR values along centerline points in the RCA, in which the x-axis is identical to the x-axis of the MPR view and the diameter/area graph. Furthermore, in 1102, the FFR values along the centerline of the volume-rendered RCA are used to color code the corresponding segments of the volume-rendered RCA. Note that the vertical markers in 1101-parts C, D correspond to the minimum area/diameter (thick line markers) and the obstruction extent (dash markers) derived from common quantitative coronary analysis techniques.

Optionally, at step 1710 of FIG. 17, a different approach is utilized to train the machine learning based artery characterization network (1704) and the machine learning based FFR pullback network (1706). This ‘end-to-end training’ is especially of value when the reference standard (1709) contains reference pullback values and single point (distal) reference values, for instance distal FFR and pullback FFR. First both networks are trained simultaneously using the pullback reference values. Thereafter, the networks are trained (using the single point reference values) individually or one of them is frozen. For instance, freezing the machine learning based FFR pullback network and hence only updating the weights of the machine learning based artery characterization network. In this example, to compute the loss, only the losses of the machine learning based FFR pullback network are used. Consequently, the machine learning based artery characterization network is optimized with respect to the final machine learning based FFR pullback network. This approach has the advantage that the machine learning based artery characterization network is optimized with respect to the actual purpose of assessing stenoses. This automatically results in focusing on the relevant segments, i.e., those where lesions are present). “Focusing” here means concretely that the contributions to the loss function and hence to the weight updates are larger for lesions. This is not the case when supervising with the lumen area, where the largest loss contributions likely come from segments with a large lumen area (which are less relevant for stenosis assessment than segments with small lumen area, i.e., lesions).

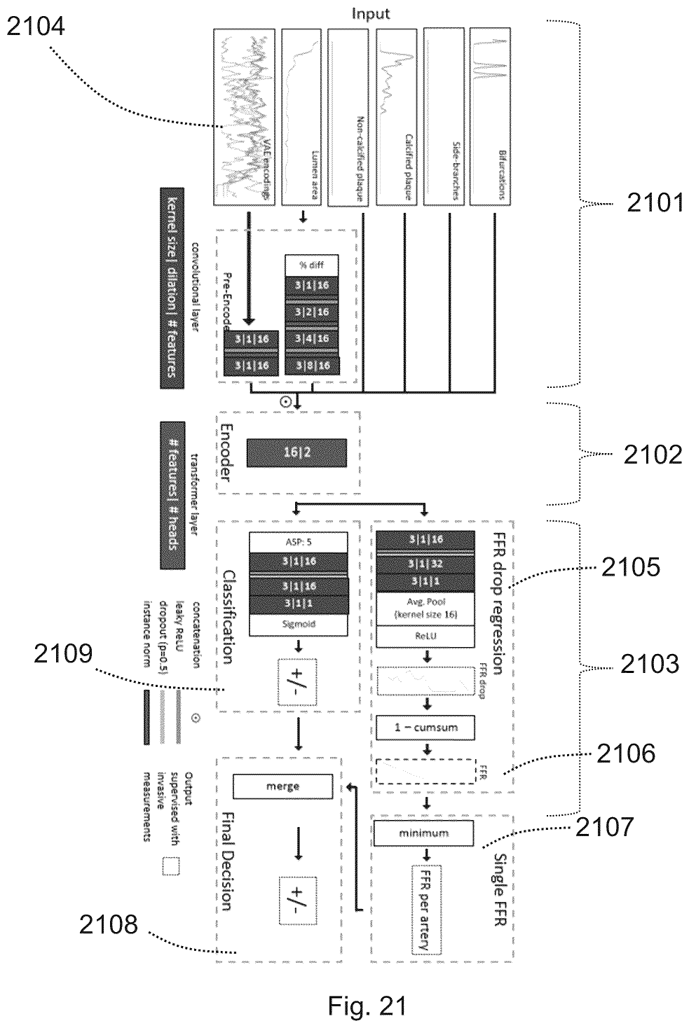

Alternatively, the machine learning based FFR pullback network is defined as a combination of the machine learning based stenosis assessment network (206, FIG. 2) with the machine learning based FFR pullback network (1706, FIG. 17). An example of such a combined network is illustrated by FIG. 21. Similar to the machine learning based stenosis assessment networks as disclosed before with reference to FIG. 2, the network of FIG. 9, 10, 13 or 14, consists of three stages. In the first stage (2101), the input as a result of the machine learning based artery characterization network (1704) and coronary tree characteristics (1705) is fed, whether or not pre-encoded, to the second stage, the encoder (2102). The main difference is that the VAE encodings (from 1704) are used as additional input (2104). In the third phase (2103), two output heads are present. The FFR drop regression head (2105), such as described with reference to FIG. 20, and a classification head (2106), such as described with reference to FIG. 9, 10, 13 or 14. The output of the network illustrated by FIG. 21 is threefold; 1) a predicted FFR pullback value (2106), 2) a single FFR value per artery (2107), and 3) a prediction of the presence of functionally significant stenosis (2108), which is based on the output of the classification head (2109) combined with the output of the single FFR prediction (2107).

Furthermore, as described by Extension 3 to the flowchart of FIG. 2, the flowchart of FIG. 17 allows to include myocardium characteristics, and the description from Extension 3 ‘include myocardium characteristics’ is also applicable for the method as described with reference to the flowchart of FIG. 17. FIG. 22 provides an illustration for integrating myocardium characteristics with respect to the machine learning based FFR pullback network architecture from FIG. 21.

### Experimental Settings

The artery characterization network (1704) underwent 1200 epochs of training, utilizing the MAE as the reconstruction loss, binary cross-entropy as the segmentation loss, and the Kullback-Leibler divergence for regularization of the latent space, each weighted with a factor of 1. To specifically improve the representation of the artery, the reconstruction loss within the reference segmentation (lumen and plaque) is weighted with a factor of 5. To supervise the lumen area regression during training, the MAE was used with a weighting of 10. The network was optimized using the ADAMW (Loshchilov and Hatter, 2019) optimizer with a learning rate of 10⁻⁵ and a batch size of 512. Once trained, we applied the network to each cross-section of the MPR to extract the lumen area and the VAE encodings along the centerline.

The FFR pullback network (1706) was trained for 150 epochs, employing the ADAMW optimizer with a linearly scheduled cyclic learning rate. The cyclic learning rate varied between 5e-4 and 1e-5 over a period of 40 epochs. Due to the limitation imposed by different artery lengths, the network could only process a single artery at a time. Hence, the loss was accumulated over eight training iterations before backpropagating, corresponding to an effective batch size of 8. To supervise the FFR pullback we counterbalance fidelity and sensitivity to noise in the pullback reference, by pooling the output of the network to a 2 mm step size using average pooling with a kernel size of 4. To minimize misregistration, invasive pullback measurements were manually registered with the input by shifting the beginning of the pullback signal such that FFR drops optimally overlap with lumen narrowing's. As the pullback reference only covers a part of the artery, pullback supervision was only applied for that part, by masking the distal overlap. The EMD loss and the histogram loss were weighted with for instance factors 0.1 and 5, respectively. Optionally the monotony loss is weighted with for instance 20. The factors were chosen based on preliminary experiments, to achieve similar magnitudes for the loss terms.

Another embodiment of the present application is now disclosed with reference to FIG. 23. The therein-depicted steps can, obviously, be performed in any logical sequence and can be omitted in parts. The method as described by the flowcharts of FIG. 2 and FIG. 17, predicts the FFR pullback by first characterizing the artery in terms of the lumen area and unsupervised features, and subsequently uses these characteristics to predict the FFR pullback. This two-step approach enables manually altering the intermediate output, i.e., the characteristics, which can be useful for correcting potential mistakes, but also to predict the FFR pullback after a successful percutaneous coronary intervention (PCI). PCI refers to a family of minimally invasive procedures used to open clogged coronary arteries (those that deliver blood to the heart). By restoring blood flow, the treatment can improve symptoms of blocked arteries, such as chest pain or shortness of breath. PCI involves combining coronary angioplasty with stenting, which is the insertion of a permanent wire-meshed tube that is either drug eluting or composed of bare metal stents. The stent delivery balloon from the angioplasty catheter is inflated with media to force contact between the struts of the stent and the vessel wall (stent apposition), thus widening the blood vessel diameter. After accessing the blood stream through the femoral or radial artery, the procedure uses coronary catheterization to visualize the blood vessels on X-ray imaging. After this, an interventional cardiologist can perform a coronary angioplasty, using a balloon catheter in which a deflated balloon is advanced into the obstructed artery and inflated to relieve the narrowing; certain devices such as stents can be deployed to keep the blood vessel open. Various other procedures can also be performed. FIG. 23 illustrates a flowchart for calculation of the FFR pullback after successful PCI treatment using the CCTA image which was acquired before treatment. As explained above, this workflow can also be used to incorporate manual adjustments of vessel characteristics resulting from the machine learning based artery characterization network (204 of FIG. 2, or 1704 of FIG. 17).

At step 2301 of FIG. 23, the method as described by the flowchart of FIG. 2, or the methods as described by the flowchart of FIG. 17 is performed.

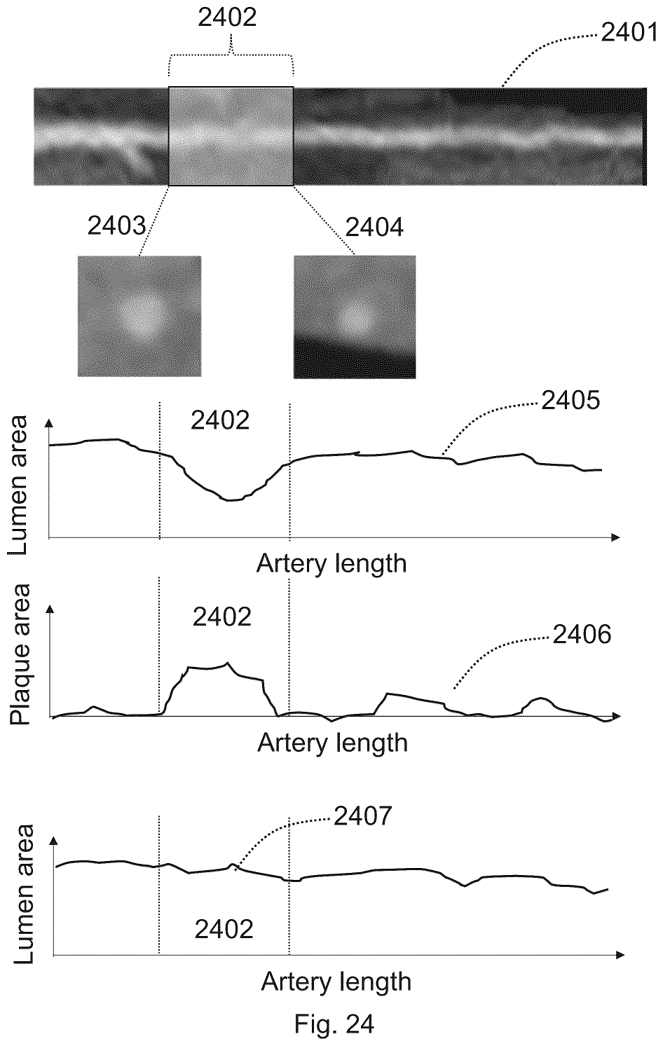

At step 2302 of FIG. 23, the derived artery characteristics (204 of FIG. 2, or 1704 of FIG. 17) are adjusted. As described above, the artery characteristics can be altered manually, semi-automatically or automatically. To simulate successful PCI, the lumen area can be enlarged in an environment around a lesion, as to imitate a placed stent, and plaque component (calcified, non-calcified, mixed) can be removed from the treated extent. FIG. 24 provides an illustration of altering the artery characteristics for simulation of successful PCI treatment. The MRI image of the vessel of interest is illustrated by 2401, in which a virtual stent placement is indicated by 2402, which also refers to the lesion extent. The cross-sectional image at the proximal side of the lesion extent is illustrated by 2403 and the cross-sectional image at the distal lesion extent is illustrated by 2404. Both cross sectional slices would represent the healthy cross-sectional area just outside the obstruction extent. Within FIG. 24 two derived artery characteristics (204 of FIG. 2, or 1704 of FIG. 17) are shown for illustration; the cross-section area along the axial trajectory of the vessel of interest (2405) and calcified plaque area along the axial trajectory of the vessel of interest (2406). Successful PCI is simulated by altering the cross-sectional area graph (2405) within the lesion extent (2402), resulting in a healthy cross-sectional area as illustrated by 2407. All plaque components within the virtual stent placement, indicated by 2402, are removed as these are not obstructing the blood flow anymore after stent placement. For instance, the calcified plaque area along the axial trajectory of the vessel of interest (2406), the part within the virtual stent (2402) can be set to zero.

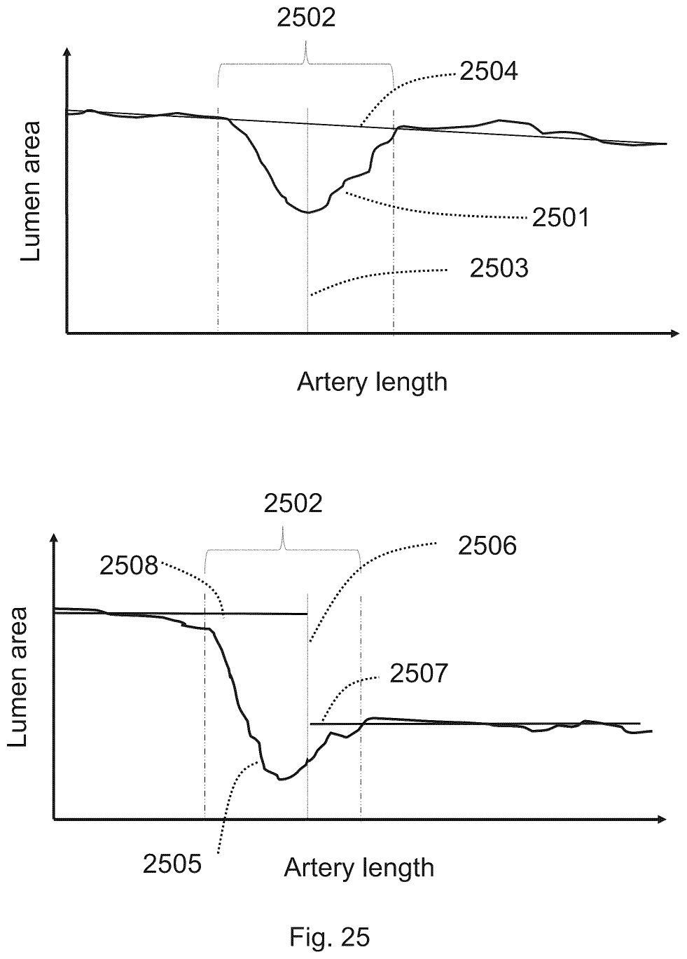

A method is now described to automatically define the lesion segment and compute the healthy reference area within the lesion segment. This will allow simulation of a successful PCI procedure and computation of the FFR pullback after such simulated PCI intervention. Within FIG. 25 the derived cross-sectional area (2501) along the axial trajectory of the vessel of interest is visualized as a result of step 204 of FIG. 2, or step 1704 of FIG. 17. The lesion extent (2502), also called obstruction extent or virtual stent extent, is for instance computed from the position of significant change in curvature of the area graph (2501) covering the minimum area (2503) as for instance taught by Kooijman et al, “Computer-aided quantitation of the severity of coronary obstructions from single view cineangiograms”, in: International Symposium on Medical Imaging and Image Interpretation. 1982: 59-64 (IEEE catalog no. 82CH1804-4). Next a straight line is fitting (2404) through the area values along the axial trajectory of the vessel of interest in which the area values within the lesion extent (2402) are excluded during the fitting as described by Gronenschild E, et al. in “CAAS II: A Second Generation system for Off-Line and On-Line Quantitative Coronary Angiography”, Cardiovascular Diagnosis 1994; 33: 61-75. The line (2504) represents the cross-section area along the axial trajectory of the vessel of interest in case the vessel is healthy, and is also called reference area along the vessel of interest. In case a branching artery (bifurcation) is present (as defined by step 205 of FIG. 2, or step 1705 of FIG. 17) within the lesion extent, a different method can be used to simulate the step-down affect in the heathy coronary after branching to compute the reference area along the vessel of interest. Within FIG. 25, 2505 shows an example of the cross-sectional area graph, in which a bifurcation is present indicated by 2506. A proximal reference area (2508) is calculated by extrapolating a fitted line (through the area values along the axial trajectory of the vessel of interest excluding all value from the proximal lesion extent) from the proximal lesion extent (2502) to the bifurcation position (2506). Likewise, a distal reference area (2507) is calculated by extrapolating a fitted a line (through the area values along the axial trajectory of the vessel of interest excluding all value from the distal lesion extent) from the distal lesion extent (2402) to the bifurcation position (2506). Furthermore, Murray's law can be incorporated within the calculation of the proximal reference area (2508) and distal reference area (2507). Murray's law described the relationship between radii at junctions in a network of fluid-carrying tubular pipes. Whenever a branch of radius r splits into two branches of radii r₁ and r₂, then all three radii should obey the equation r³=r₁³+r₂³.

An alternative automatic method to simulate virtual stent placement is now described with reference to FIG. 26 and utilizes the dense latent space of the variational autoencoder. Within FIG. 26, the MRI image of the vessel of interest is illustrated by 2601, in which a virtual stent placement is indicated by 2602. The cross-sectional image at the proximal side of the lesion extent is illustrated by 2603 and the cross-sectional image at the distal lesion extent is illustrated by 2604. Both cross sectional slices would represent the healthy cross-sectional area just outside the obstruction extent. Within graph 2605 of FIG. 26, the cross-sectional area (2609) as a result of the artery characterization network (1704 of FIG. 17) is shown. Furthermore, the FFR pullback (2610), as a result of 1706 of FIG. 17, is shown.

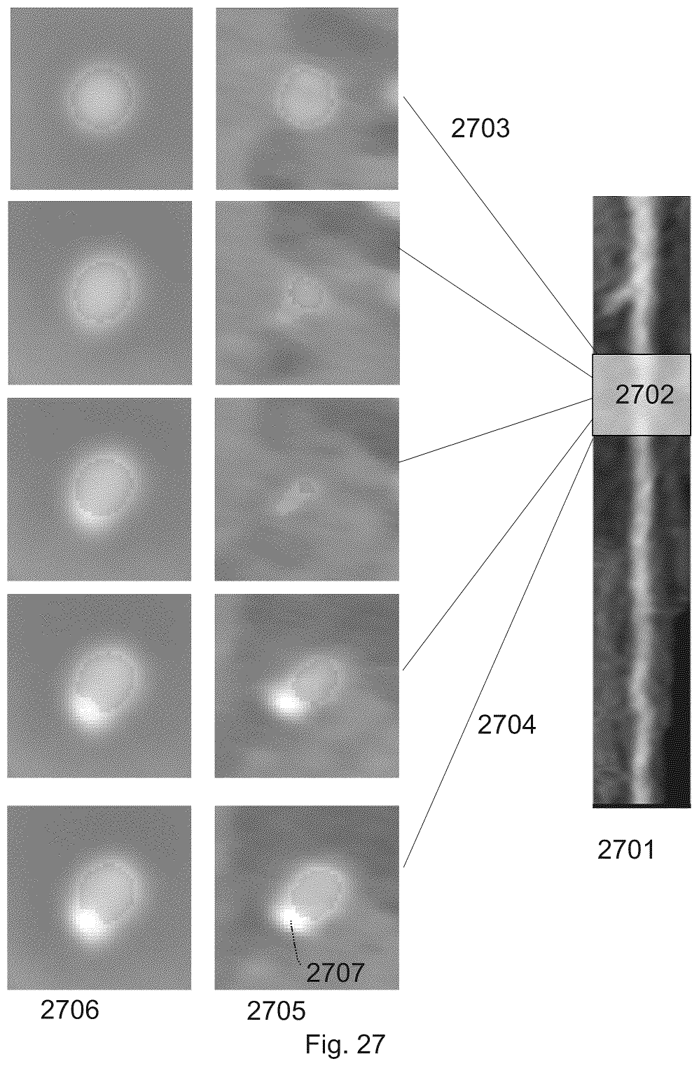

As described above with reference to FIGS. 19a and 19b, the variational autoencoder can be trained to extract relevant information from the feature vector (z). From this VAE feature vector, features (z) are used as input to a linear layer that directly regresses the lumen area (see the text with reference to 1906 of FIG. 19a). To obtain unsupervised encodings that represent a lesion after treatment, linear interpolate between the encodings (1907 of FIG. 19a) of the healthy segments in front (proximal) of the lesion and behind (distal) of the lesion. As the encoding space of a VAE is dense, the interpolated encodings correspond to realistic coronary artery segments. Furthermore, the disentangled nature of the encodings ensures smoothness of the transition. The interpolated encodings are fed to the lumen area regression network (1906), to obtain the cross-section area graph (2613) incorporating virtual treatment. This is further illustrated by FIG. 26. Picture 2606b illustrates the cross-sectional slice from the MPR image (2601) at the proximal position of the lesion extent (2602), likewise picture 2608b illustrates the cross-sectional slice from the MPR image (2601) at the distal position of the lesion extent (2602). The picture 2607b shown as a cross-sectional slice from the MPR image (2601) within the lesion segment (2601). Within this slice, the lumen is represented by 2611, and plaque is represented by 2612. To illustrate the behavior of interpolation in latent space, pictures 2606a, 2607a and 2608a show the results of the interpolation of the encodings. The image itself is a result of the decoder (1904) which is trained to reconstruct the central input slice of the input stack from the feature vector (z), and the overlay within these pictures is a result of the segmentation decoder (1905). In case the virtual stent does not cover the full disease extent, the effect is this ‘incorrect’ treatment can be simulated as well using the described interpolation in feature space. This is illustrated with reference to FIG. 27. The MPR of the vessel of interest is shown by 2701, in which a virtual stent placement is indicated by 2702. In this example, the proximal (2703) slice does not show disease but the distal side (2704) of the virtual stent still shown plaque disease. At distal cross-sectional slice, calcified plaque is present (2707). The cross-sectional slices represented by column (2705), shown the cross-sectional slice with overlay along the length of the virtual stent placement. The cross-sectional slices represented by column (2706), shown the results of the interpolation of the encodings. The image itself is a result of the decoder (1904) which is trained to reconstruct the central input slice of the input stack from the feature vector (z), and the overlay within these pictures is a result of the segmentation decoder (1905). As in the example the virtual stent does not cover the full disease extent, calcified plaque component distally is still present in the simulated healthy cross-sectional slices at the distal part of the virtual stent. Alternatively, instead of linear interpolate between the encodings (1907 of FIG. 19a) of the healthy segments in front (proximal) of the lesion and behind (distal) of the lesion, a linear interpolate is performed between the feature vector (z) of the healthy segments in front (proximal) of the lesion and behind (distal) of the lesion.

Finally at step 2303 of FIG. 23, the FFR pullback after virtual stent placement is calculated. Within step 2302, the vessel characteristics which simulate virtual stent placement have been calculated. Either the adjusted vessel characteristics are fed to the machine learning based stenosis assessment network of step 206 from FIG. 2, or the adjusted vessel characteristics are fed to the machine learning based FFR pullback network of step 1706 of FIG. 17 to calculate the FFR and/or FFR pullback simulating virtual stent placement. An example of feeding the adjusted vessel characteristics (2302) to the machine learning based FFR pullback network of step 1706 of FIG. 17 is provided by picture 2606 of FIG. 26, the FFR pullback graph after virtual stent placement is shown by 2611. This example shows the elimination of the FFR drop within the lesion segment (2602) when comparing to the FFR pullback graph before treatment (2610).

Alternatively virtual stent placement can be simulated without adjusting of the vessel characteristics (2302), and directly adjust the MPR of the vessel of interest within the workflow of FIG. 2 or FIG. 17. First, the lesion extent is determined from the lumen area graph, for instance as described before with reference to FIG. 25. Next, the MPR slices covering the lesion extent within the MPR of the vessel of interest. For instance, with reference to FIG. 26 picture 2601 showing the MPR of the vessel of interest, by interpolating between the healthy cross-sectional MRI slices 2603 and 2604, to create a healthy MPR image along the axial trajectory of the vessel of interest. Such an interpolating can also be performed by means of a deep learning network, which is trained to generate/interpolate new cross-sectional slices from a MPR image between a healthy proximal cross-section slice and healthy distal cross-sectional slice from such MPR image. Finally, this new MPR of the vessel of interest is fed to the machine learning based artery characterization network (204 of FIG. 2, or 1704 of FIG. 17) and executing the remaining steps of FIG. 2 or FIG. 17.

Another embodiment of the present application is now disclosed with reference to FIG. 28. The therein-depicted steps can, obviously, be performed in any logical sequence and can be omitted in parts. FIG. 28 illustrates a flowchart of a machine learning based method for automatically coronary tree extraction and anatomical labeling of the coronary artery tree.

FIG. 29 provides a high-level overview of a machine learning based method for automatically extracting a coronary tree in CCTA images by iteratively tracking automatically placed seed points. Subsequently, an ensemble of graph convolutional neural networks (GCNs) is used to refine the extracted tree and to label its segments. On high-level, with reference to FIG. 29, after initialization (2901) with the seeds (2905) and ostia (2906), the coronary tree is extracted by 1) iteratively tracked (2902), whereafter 2) an ensemble of GCN's is applied to refine the initially extracted tree (2903). Finally, another GCN's is configured to label the anatomical segments (2904).

Within step 2801 of FIG. 28, a CCTA image dataset is obtained. Such an image dataset represents a volumetric CCTA image dataset, for instance a single contrast enhanced CCTA dataset. This CCTA dataset can be obtained from a digital storage database, such as an image archiving and communication system (PACS) or a VNA (vendor neutral archive), a local digital storage database, a cloud database, or acquired directly from a CT imaging modality. During the CCTA imaging, a contrast agent was induced in the patient. Furthermore, the CCTA imaging can be ECG triggered.

The coronary tree is represented as an undirected tree graph. Each point in the centerline corresponds to a node in the graph and the connections between centerline points are represented by undirected edges. Within step 2802 of FIG. 28, the processors initialize the tree graph. The tree graph is initialized (2901) by automatically placing seed points in the coronary arteries (2905) and left and right coronary ostia (2906). Subsequently, the tree graph is built directly during coronary artery extraction by simultaneously tracking coronary centerlines (2907) from the identified seed points. In the process, new points in the coronary arteries are appended iteratively to the tree graph. In this way redundantly tracking sections multiple times is prevented and computational redundancy is reduced.

For initialization, seed points and the location of the coronary ostia are predicted by two fully convolutional neural networks (seed-CNN and ostia-CNN). The architecture, identical for both networks, comprises seven 3D convolutional layers with kernel width of three. In layers 1-4, the number of channels is set to 32 and in layers 5 and 6 set to 64. The final layer yields a single output channel. To increase the receptive field, in layers 3 and 4, dilation factors of two and four are used, respectively, while in the remaining layers the dilation is set to one. The seed-CNN and ostia-CNN are trained to predict for each voxel the negative exponential of the distance to the nearest coronary artery centerline or ostia, respectively. This renders a heatmap-like prediction map indicating where the coronary arteries and ostia are located. Thereafter, seed points are identified as local maxima from the predicted heatmaps.

Within step 2803 of FIG. 28, the processor performs the coronary tree tracking. To add new nodes to the tree, a CNN (tracking-CNN) is used to predict a direction and a step size from each end point of the graph. The tracking-CNN receives a 3D image patch at the seed point location and predicts a direction in the form of binary outputs for discrete, evenly spaced locations on the unit sphere, as well as the radius of the coronary artery. To obtain a new point, a step is taken into the predicted direction with the step size corresponding to the vessel radius prediction. To prevent backwards direction prediction, the predicted direction classes close to the followed direction are masked. The architecture of the tracking-CNN is identical to the architecture of the seed-CNN and ostia-CNN (as described by step 2802), apart from the number of output nodes in the seventh layer. The tracking CNN uses 501 output channels: 500 direction classes and one additional channel for the radius regression. Outputs for the direction classes are subsequently combined using a softmax activation layer.

Only the end points are tracked, i.e., nodes with fewer than two edges. Among these nodes, only those where the uncertainty of the direction prediction is below a certain threshold are tracked. Because nodes that are not connected to the ostia are less likely to reside in coronary arteries, a different uncertainty threshold for nodes that are connected to the ostia than for all other nodes are employed. The uncertainty is given by the entropy over the direction output classes.

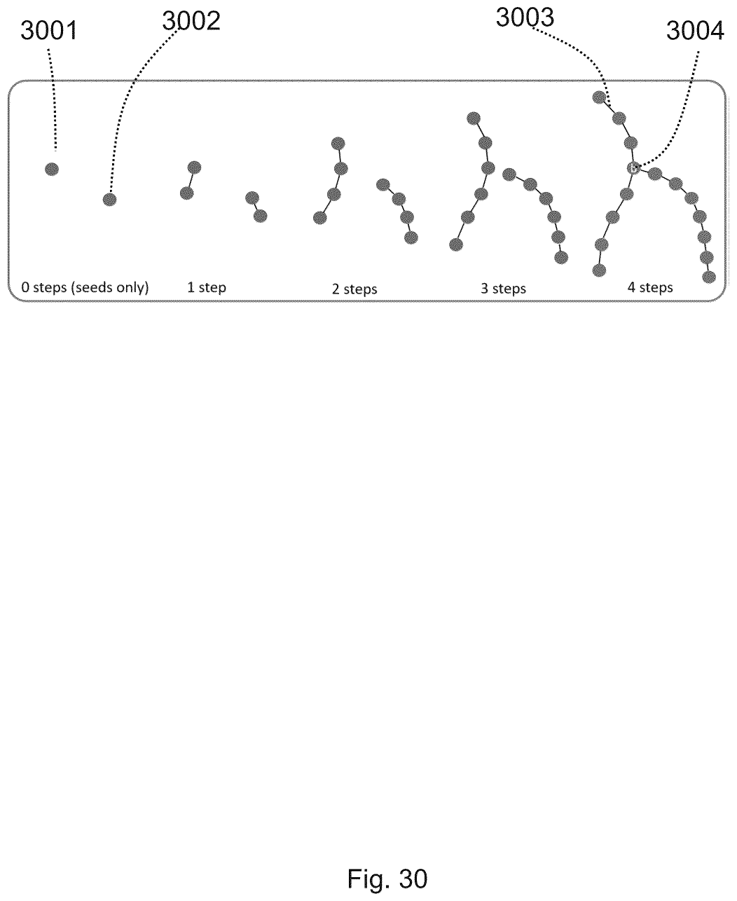

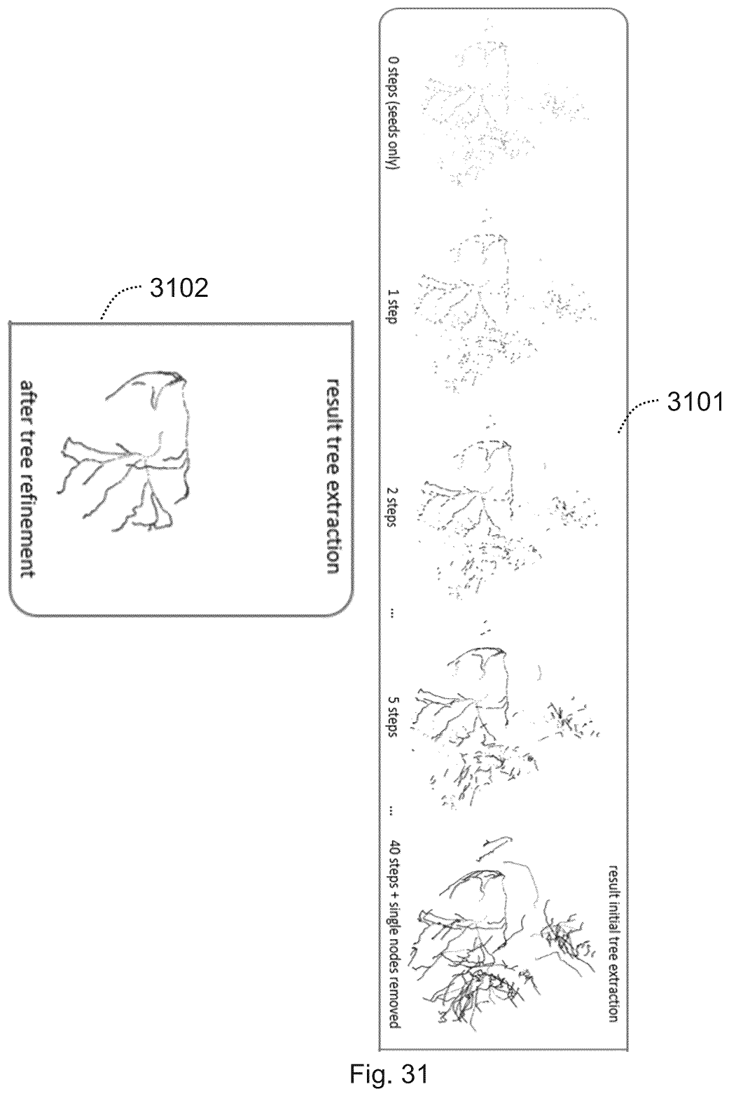

Simultaneous tracking of all seed points yields multiple sub-graphs. Hence, these tracked sub-graphs are merged when overlapping. An example of the simultaneous tracking is provided in FIG. 30, where tracking of two seed points blue node (3001) and red node (3002) is illustrated. As the two tracked sub-graphs (blue and red nodes) overlap after 3 steps, they are merged into a single connected graph (green nodes, 3003) in step 4. Note that this merging avoids redundant tracking of proximal nodes, which are located above the bifurcation (3004). Therefore, a new node is created only when its location is not already occupied by another node, i.e., the node is not part of another sub-graph. The node overlap is assessed using the artery radius at each existing node as predicted by the tracker. FIG. 31, 3101 illustrates the building of the tree graph that includes the extraction of the seeds and subsequent artery tracking.

Within step 2804 of FIG. 28, the coronary tree is refined. During tree tracking as described by step 2803, a high sensitivity is archived to obtain a complete coronary artery tree (2902). However, this may come at the cost of limited precision, i.e., false positive extractions representing veins or other tubular structures. Therefore, to remove false positives from the extracted tree, an ensemble of GCNs (2908) is used. GCNs are a generalization of CNNs to arbitrary graph inputs. Therefore, GCNs enable learning combined representations of node features and connectivity's directly from graphs. Hence, it is needed to define features that can subsequently be used by a GCN.

To enable refinement of the initially extracted trees, artery segments are created by grouping adjacent centerline points of the tree graph. These segments are characterized using a set of features describing location, orientation, geometry, and image appearance of the segments.

The location of the segment is described by the Cartesian coordinates of the segment's centerline points at the start, the end and every quartile of the segment's length, with respect to the center of the left ventricle myocardium as for instance taught by Bruns et al, “Deep learning from dual-energy information for whole-heart segmentation in dual-energy and single-energy non-contrast-enhanced cardiac CT”, in: Medical Physics 47, 2020, pp. 5048-5060. Two features that describe the segment's orientation are extracted. The first feature corresponds to the Cartesian coordinates of the normalized directional vectors between the segment end points. The second feature consists of the Cartesian coordinates of the normalized directional vectors between the first two centerline points in the segment. To describe the geometry of the vessel, the mean and standard deviation over the vessel radii of the centerline points in a segment are used, corresponding to the vessel size.

The appearance of the segment is characterized by its texture, derived using the outputs of the tracking-CNN (2907, and described by step 2803) and the seed-CNN (2905, and described by step 2802). This characterization is described by the mean and the standard deviation over the entropy values of the centerline points in a segment. The entropy value corresponds to the uncertainty of the tracker at that location. Intuitively, this indicates the extent to which the segment resembles a vessel. Similarly, the mean and standard deviation over the output values of the seed-CNN is calculated, extracted from the output map at the centerline point locations in a segment.

To distinguish real coronary artery segments (positive class) from other vessel-like structures (negative class), binary classification is performed using GCNs. GCNs enable learning combined representations of node features and connections using the initially extracted tree graph directly as input for classification. To increase robustness to potentially missing segments, an ensemble of GCNs is used and applied to multiple graphs of different resolution.

In graph attention networks (GATs), weights for the aggregation of features from neighboring nodes are learned end-to-end utilizing attention sub-networks. The aggregation function of a GAT is defined as \vec{h}ᵢ′=σ( [symbol] αᵢⱼW\vec{h}ⱼ), with \vec{h}ⱼ as input features of nodes in the local neighborhoods [symbol] ᵢ that are first transformed by the weight matrix W. σ denotes a nonlinearity, for which the LeakyReLU function is used. To determine the weighting coefficients αᵢⱼ attenuation subnetworks (heads) are used, which are parameterized by weight vectors \vec{a}, as

$${α}_{i j} = \frac{exp ( σ ( {\vec{a}}^{T} [ W {\vec{h}}_{i} ‖ W {\vec{h}}_{j} ] ) )}{{Σ}_{k ∈ {ℵ}_{i}} exp ( σ ( {\vec{a}}^{T} [ W {\vec{h}}_{i} ‖ W {\vec{h}}_{k} ] ) )} , \qquad ( equation 7 )$$

where ∥ denotes concatenation.
   As the attention mechanism enables the GAT to express the importance of neighboring segments for one another, they are likely a suitable choice for encoding local segment neighborhoods to refine our extracted coronary artery trees.

To leverage information about the geometrical structure of the coronary artery tree, a sufficiently large receptive field is needed. However, GCNs typically have a limited receptive field as taught by Wu et al, “A Comprehensive Survey on Graph Neural Networks.”, IEEE Transactions on Neural Networks and Learning Systems 32, 2021, pp. 4-24. Hence, the effective receptive field is increased by using coarser graphs at lower resolutions as input. To create the coarse graphs, adjacent nodes of the initially extracted tree (fine graph) are grouped into segments.

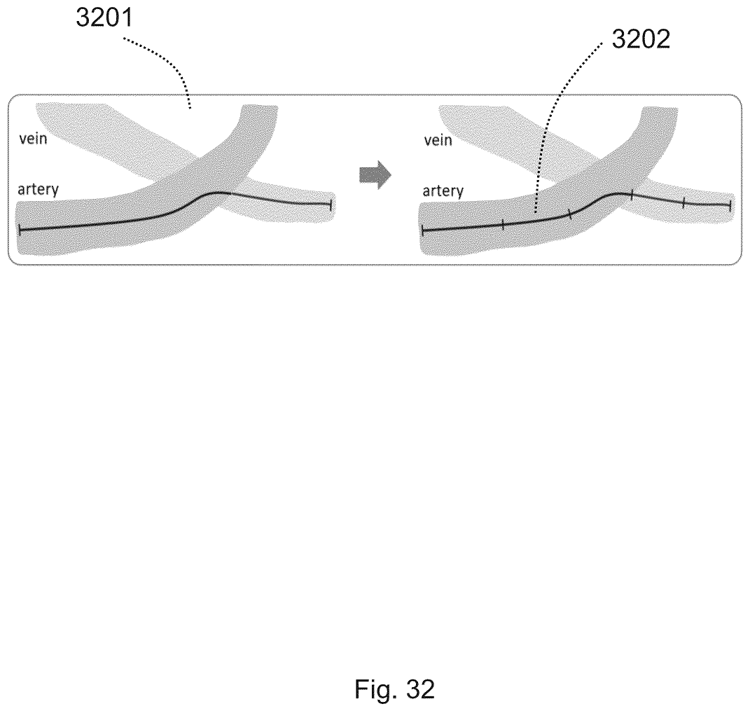

While grouping nodes increases the receptive field, it can also group true and false nodes into the same segment, e.g., when an artery leaks into a vein, resulting in label ambiguity, see FIG. 32, 3201. Therefore, multiple coarse graphs (3202) are created by grouping the centerline nodes into segments in two different manners. For the first course graph, we apply the conventional definition as taught by Hampe et al., “Graph attention networks for segment labeling in coronary artery trees”, Medical Imaging 2021: Image Processing, in which segments are separated by bifurcations. To enable subdivision of these segments, additional graphs are created by dividing the segments from the undivided graph above into smaller segments with lower resolutions. To enable information flow between all segments adjacent to a bifurcation, the edges of the graphs are established by combining all possible pairs of involved segments. Furthermore, information flow from each node to itself is enabled by inserting an edge from each node to itself.

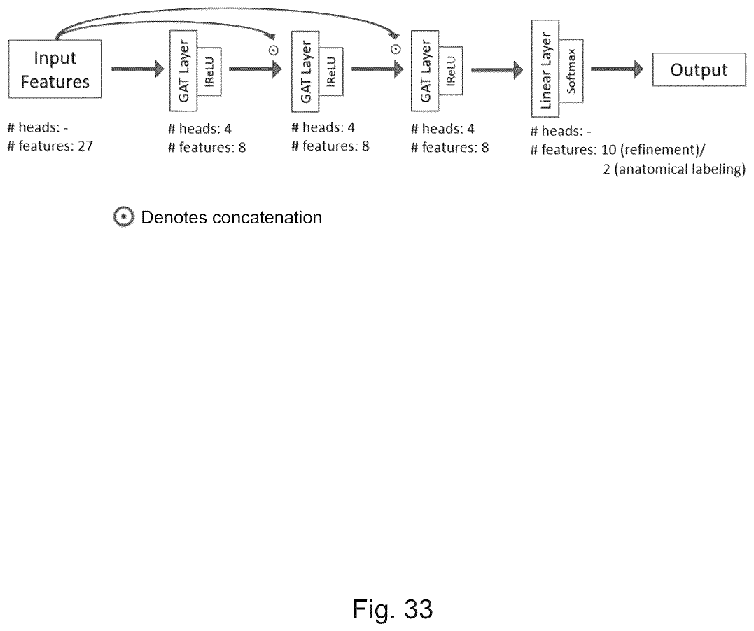

To combine the benefits of reduced label ambiguity (graphs with small segments) and the large receptive field (graphs with large segments), a multiresolution graph ensembling strategy is employed. For this, the predictions for all graphs with different resolutions are back projected to the fine graph. Ensembling was performed by taking the average over the output probabilities of all GCNs on the fine graph.

Whereas the GCNs in the ensemble are applied to graphs with different resolutions, the network architecture, shown schematically in FIG. 33, is identical for all GCNs. It is comprised of three GAT layers with dense connections from the input features to all consecutive GAT layers. In each GAT layer, four heads with eight encodings per head are used in combination with residual connections and dropout. Additionally, the input features are concatenated to the input of each consecutive GAT layer. Finally, binary output probabilities, corresponding to true segments of the coronary artery tree and other vessel-like structures, are predicted using a linear layer followed by the softmax activation function. Picture 3102 of FIG. 31 provides an illustration of the result of the tree refinement step.

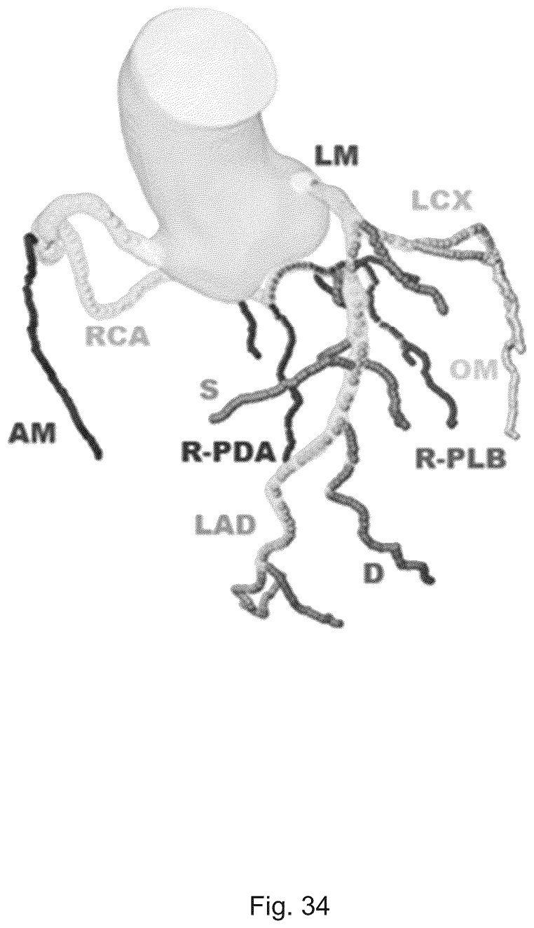

Finally, at step 2805 of FIG. 28, the anatomical coronary labeling is performed. Anatomical labels (are assigned to the segments of the extracted coronary artery tree (2904). Similar to tree extraction, an ensemble of GCNs (2909) trained on graphs with different resolutions for anatomical labeling is employed. Segment labeling is posed as a multiclass classification task. By using GCNs, the model is able to learn information about individual segments and about the relation between these segments. Moreover, the multi-resolution ensemble approach mitigates the problems related to label ambiguity due to missing bifurcations, while ensuring the receptive field of the method is large enough to model inter-segment relationships. The different resolution graphs are obtained by grouping centerline points into segments similar to the method as described at step 2804. The network architecture for anatomical labeling is identical to the one for tree refinement (step 2804), with the exception that for anatomical labeling ten output classes are used, one for each coronary segment label (from the reference standard 2806). Furthermore, the same input features are employed. FIG. 34 provides an illustration of the result of step 2805; the figure shows the extracted coronary tree in which each coronary segment is anatomically labelled.

The reference standard (2806) is a database which contains data of multiple patients. Each set within the database contains for each patient a) contrast enhanced CT image datasets (2801 represents reference image sets during the training phase) and the corresponding coronary artery centerline trees in which each centerline point within the tree is assigned with the lumen radius and an anatomical label as for instance according to the model as introduced by Austen et al. “A reporting system on patients evaluated for coronary artery disease. Report of the Ad Hoc Committee for Grading of Coronary Artery Disease, Council on Cardiovascular Surgery, American Heart Association”, Circulation 1975; 51, 5-40.

### Experimental Settings

All GCNs were trained using the reference standard (2806) for 500 epochs by using the Adam optimizer. Unless specified differently, the learning rate is set to 0.001 for the first 300 epochs and set to 0.0001 for the remaining 200 epochs. For dropouts, a probability of 0.2 is used.

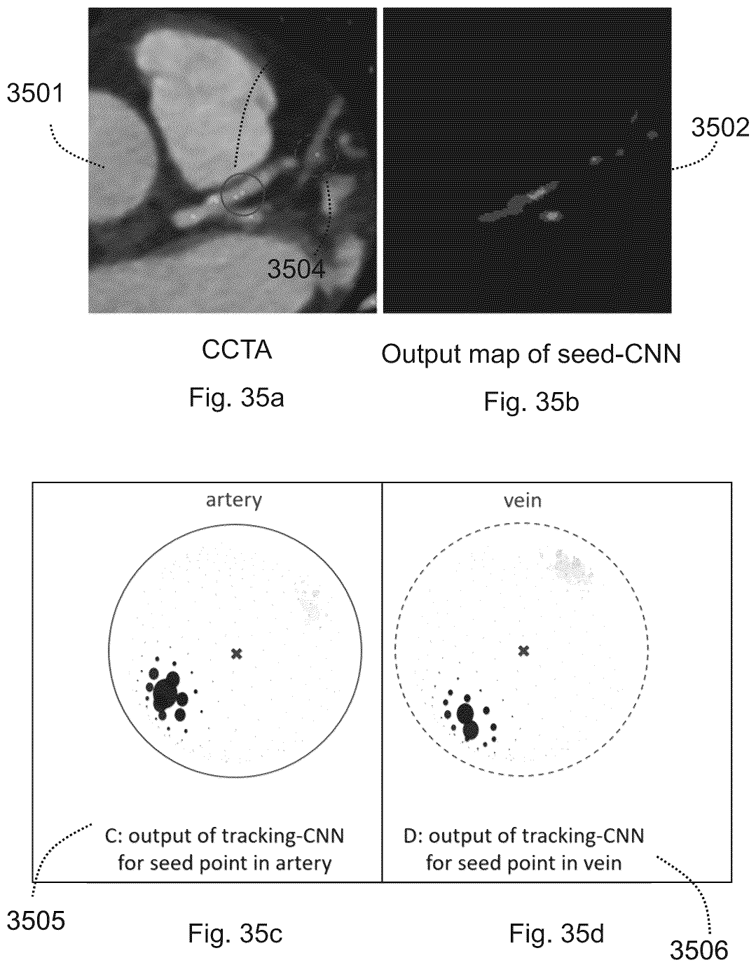

Tree Initiation and Tree Tracking (2802, 2803): All three CNN's (2905, 2906 and 2907) are trained using the reference standard (2806) as described by Wolterink et al, “Coronary artery centerline extraction in cardiac CT angiography using a CNN-based orientation classifier”, Med Image Anal. 2019 January; 51:46-60. For initialization of the tracking, 400 seed points are used. We chose such a large number of seed points to ensure high sensitivity. Moreover, we reduced the redundancy and ensured that seed points were not generated too close to one another by enforcing a minimum distance of 3 mm between seed points. For artery tracking, the input for the tracking-CNN was a 19×19×19 image patch and the number of output directions was 500. We tracked nodes connected to the ostia if the entropy over the output directions, i.e., uncertainty, was below 0.9, and nodes disconnected from the ostia if the entropy was below 0.7. To avoid backward tracking, we masked directions within less than 60 degrees of previously tracked directions. Tracking terminated if no nodes remained to be tracked or if a maximum number of 40 steps—determined in preliminary experiments—was reached, preventing extensive false positive extractions. In comparison to tracking proximal sections redundantly, the proposed graph tracking reduces the number of tracked nodes from on average 7,667 to 4,476 (42%). After tracking was finished, we removed all single nodes, as they presented seed points that have not been tracked. Given that the extracted seed points provide the starting point for the construction of the tree, incorrect seed points and leakages due to incorrect centerline direction prediction can cause false positives, typically in artery-like structures like veins. Picture 3501 and 3502 of FIG. 35 illustrate that the seed-CNN outputs high values for coronary arteries and lower values for coronary veins. Picture 3501 shows an axial slice of the CCTA image dataset, displaying coronary arteries (3503) and veins (3504) side by side. Picture 3502 shows the output map of the seed-CNN with high probability values for coronary arteries and lower probability values for coronary veins. As the tracker was trained on relatively small image patches around centerline voxels, it tracks coronary arteries (3505), but it also tracks other vessel-like structures, such as coronary veins (3506). Picture 3505 shows the output of the tracking-CNN for a patch around a seed point in a coronary artery (location in the CCTA marked by a circle 3503). Probability output is visualized as activations on the unit sphere, with size of the activations corresponding to the probability magnitude. Picture 3506 shows the output of the tracking-CNN for a patch around a seed point in a coronary vein (location in the CCTA marked by a dashed circle 3504). Therefore, after tree tracking, we subsequently refined the tree by removing false positives (2804).

Tree Refinement (2804): To reduce label ambiguity (FIG. 32) and nevertheless learn from a large receptive field, we used a graph ensembling strategy based on coarse graphs with different resolutions. Specifically, in addition to the undivided graph, we created graphs with a predefined centerline points per segment, for instance 10.

Anatomical Labeling (2805): To learn robustness of anatomical labeling to potential errors from tree extraction, instead of the reference trees from the reference standard (2806) we used the automatically extracted trees (result of step 2804) as input to the GCNs for training. To train the ensemble for anatomical labeling, automatically extracted trees were represented by the same coarse graphs as for tree extraction. Preferable multiple graphs are created with a number of predefined centerline points per segment, for instance 5, 10, 20 and 30. Therefore, to set the reference for training the GCNs we projected the anatomical labels from the reference trees onto the automatically extracted coarse graphs. During training of anatomical labeling, nodes that were present in the automatically extracted tree but not in the reference tree (false positive nodes) were not backpropagated.

The present disclosure mainly describes the organ of interest as the myocardium and the vessels being the coronary arteries. The skilled person would appreciate that this teaching can be equally extended to other organs. For instance, the organ of interest can be the kidney, which is perfused by the renal arteries, or (parts) of the brain as perfused by the intracranial arteries. Furthermore, the present disclosure refers to CCTA datasets (in several forms). The skilled person would appreciate that this teaching can be equally extended to other imaging modalities, for instance rotational angiography, MRI, SPECT, PET, Ultrasound, X-ray, or the like.

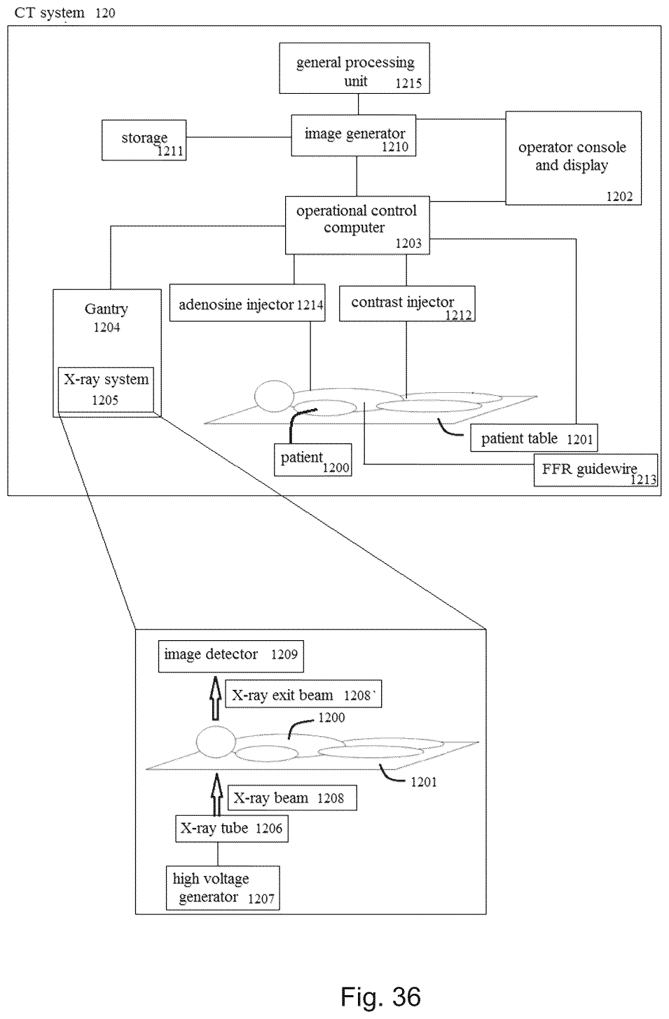

The embodiment of this disclosure can be used on a standalone system or included directly in, for instance, a computed tomography (CT) system. FIG. 36 illustrates an example of a high-level block diagram of a computed tomography (CT) system. In this block diagram the embodiment is included as an example how the present embodiment could integrate in such a system.

Portions of the system (as defined by various functional blocks) may be implemented with dedicated hardware, analog and/or digital circuitry, and/or one or more processors operating program instructions stored in memory.

The most common form of computed tomography is X-ray CT, but many other types of CT exist, such as dual-energy, spectral multi-energy, or photon-counting CT. Also, positron emission tomography (PET) and single-photon emission computed tomography (SPECT) or combined with any previous form of CT.

The CT system of FIG. 36 describes an X-ray CT system. In an X-ray CT system an X-ray system moves around a patient in a gantry and obtains images. Through use of digital processing a three-dimensional image is constructed from a large series of two-dimensional angiographic images taken around a single axis of rotation.

For a typical X-ray CT system 120 an operator positions a patient 1200 on the patient table 1201 and provides input for the scan using an operating console 1202. The operating console 1202 typically comprises of a computer, a keyboard/foot paddle/touchscreen and one or multiple monitors.

An operational control computer 1203 uses the operator console input to instruct the gantry 1204 to rotate but also sends instructions to the patient table 1201 and the X-ray system 1205 to perform a scan.

Using a selected scanning protocol selected in the operator console 1202, the operational control computer 1203 sends a series of commands to the gantry 1204, the patient table 1201 and the X-ray system 1205. The gantry 1204 then reaches and maintains a constant rotational speed during the entire scan. The patient table 1201 reaches the desired starting location and maintains a constant speed during the entire scan process.

The X-ray system 1205 includes an X-ray tube 1206 with a high voltage generator 1207 that generates an X-ray beam 1208.

The high voltage generator 1207 controls and delivers power to the X-ray tube 1206. The high voltage generator 1207 applies a high voltage across the vacuum gap between the cathode and the rotating anode of the X-ray tube 1206.

Due to the voltage applied to the X-ray tube 1206, electron transfer occurs from the cathode to the anode of the X-ray tube 1206 resulting in X-ray photon generating effect also called Bremsstrahlung. The generated photons form an X-ray beam 1208 directed to the image detector 1209.

An X-ray beam 1208 comprises of photons with a spectrum of energies that range up to a maximum determined by among others the voltage and current submitted to the X-ray tube 1206.

The X-ray beam 1208 then passes through the patient 1200 that lies on a moving table 1201. The X-ray photons of the X-ray beam 1208 penetrate the tissue of the patient to a varying degree. Different structures in the patient 1200 absorb different fractions of the radiation, modulating the beam intensity.

The modulated X-ray beam 1208′ that exits from the patient 1200 is detected by the image detector 1209 that is located opposite of the X-ray tube.

This image detector 1209 can either be an indirect or a direct detection system.

In case of an indirect detection system, the image detector 1209 comprises of a vacuum tube (the X-ray image intensifier) that converts the X-ray exit beam.

1208′ into an amplified visible light image. This amplified visible light image is then transmitted to a visible light image receptor such as a digital video camera for image display and recording. This results in a digital image signal.

In case of a direct detection system, the image detector 1209 comprises of a flat panel detector. The flat panel detector directly converts the X-ray exit beam 1208′ into a digital image signal.

The digital image signal resulting from the image detector 1209 is passed to the image generator 1210 for processing. Typically, the image generation system contains high-speed computers and digital signal processing chips. The acquired data are preprocessed and enhanced before they are sent to the display device 1202 for operator viewing and to the data storage device 1211 for archiving.

In the gantry the X-ray system is positioned in such a manner that the patient 1200 and the moving table 1201 lie between the X-ray tube 1206 and the image detector 1209.

In contrast enhanced CT scans, the injection of contrast agent must be synchronized with the scan. The contrast injector 1212 is controlled by the operational control computer 1203.

For FFR measurements an FFR guidewire 1213 is present, also adenosine is injected by an injector 1214 into the patient to induce a state of maximal hyperemia.

An embodiment of the present application is implemented by the X-ray CT system 120 of FIG. 18 as follows. A clinician or other user acquires a CT scan of a patient 1200 by selecting a scanning protocol using the operator console 1202. The patient 1200 lies on the adjustable table 1201 that moves at a continuous speed during the entire scan controlled by the operational control computer 1203. The gantry 1204 maintains a constant rotational speed during the entire scan.

Multiple two-dimensional X-ray images are then generated using the high voltage generator 1207, the X-ray tube 1206, the image detector 1209 and the digital image generator 1210 as described above. This image is then stored on the hard drive 1211. Using these X-ray images, a three-dimensional image is constructed by the image generator 1210.

The general processing unit 1215 uses the three-dimensional image to perform the classification as described above.

There have been described and illustrated herein several embodiments of a method and apparatus for automatically identifying patients with functionally significant stenosis, based on the information extracted from a single CCTA image only.

While particular embodiments of the present application have been described, it is not intended that the present application be limited thereto, as it is intended that the present application be as broad in scope as the art will allow and that the specification be read likewise.

For example, multi-phase CCTA datasets can be used, functional assessment of renal arteries in relation to the perfused kidney can be assess based on the methodology disclosed, the data processing operations can be performed offline on images stored in digital storage, such as a PACS or VNA in DICOM (Digital Imaging and Communications in Medicine) format commonly used in the medical imaging arts. It will therefore be appreciated by those skilled in the art that yet other modifications could be made to the provided application without deviating from its spirit and scope as claimed.

The embodiments described herein may include a variety of data stores and other memory and storage media as discussed above. These can reside in a variety of locations, such as on a storage medium local to (and/or resident in) one or more of the computers or remote from any or all of the computers across the network. In a particular set of embodiments, the information may reside in a storage-area network (“SAN”) familiar to those skilled in the art.

Similarly, any necessary files for performing the functions attributed to the computers, servers or other network devices may be stored locally and/or remotely, as appropriate.

Where a system includes computerized devices, each such device can include hardware elements that may be electrically coupled via a bus, the elements including, for example, at least one central processing unit (“CPU” or “processor”), at least one input device (e.g., a mouse, keyboard, controller, touch screen or keypad) and at least one output device (e.g., a display device, printer, or speaker). Such a system may also include one or more storage devices, such as disk drives, optical storage devices and solid-state storage devices such as random-access memory (“RAM”) or read-only memory (“ROM”), as well as removable media devices, memory cards, flash cards, etc.

Such devices also can include a computer-readable storage media reader, a communications device (e.g., a modem, a network card (wireless or wired), an infrared communication device, etc.) and working memory as described above.

The computer-readable storage media reader can be connected with, or configured to receive, a computer-readable storage medium, representing remote, local, fixed and/or removable storage devices as well as storage media for temporarily and/or more permanently containing, storing, transmitting, and retrieving computer-readable information. The system and various devices also typically will include a number of software applications, modules, services, or other elements located within at least one working memory device, including an operating system and application programs, such as a client application or web browser.

It should be appreciated that alternate embodiments may have numerous variations from that described above. For example, customized hardware might also be used and/or particular elements might be implemented in hardware, software (including portable software, such as applets) or both.

Further, connection to other computing devices such as network input/output devices may be employed.

Various embodiments may further include receiving, sending, or storing instructions and/or data implemented in accordance with the foregoing description upon a computer-readable medium. Storage media and computer readable media for containing code, or portions of code, can include any appropriate media known or used in the art, including storage media and communication media, such as, but not limited to, volatile and non-volatile, removable and non-removable media implemented in any method or technology for storage and/or transmission of information such as computer readable instructions, data structures, program modules or other data, including RAM, ROM, Electrically Erasable Programmable Read-Only Memory (“EEPROM”), flash memory or other memory technology, Compact Disc Read-Only Memory (“CD-ROM”), digital versatile disk (DVD) or other optical storage, magnetic cassettes, magnetic tape, magnetic disk storage or other magnetic storage devices or any other medium which can be used to store the desired information and which can be accessed by the system device. Based on the disclosure and teachings provided herein, a person of ordinary skill in the art will appreciate other ways and/or methods to implement the various embodiments.

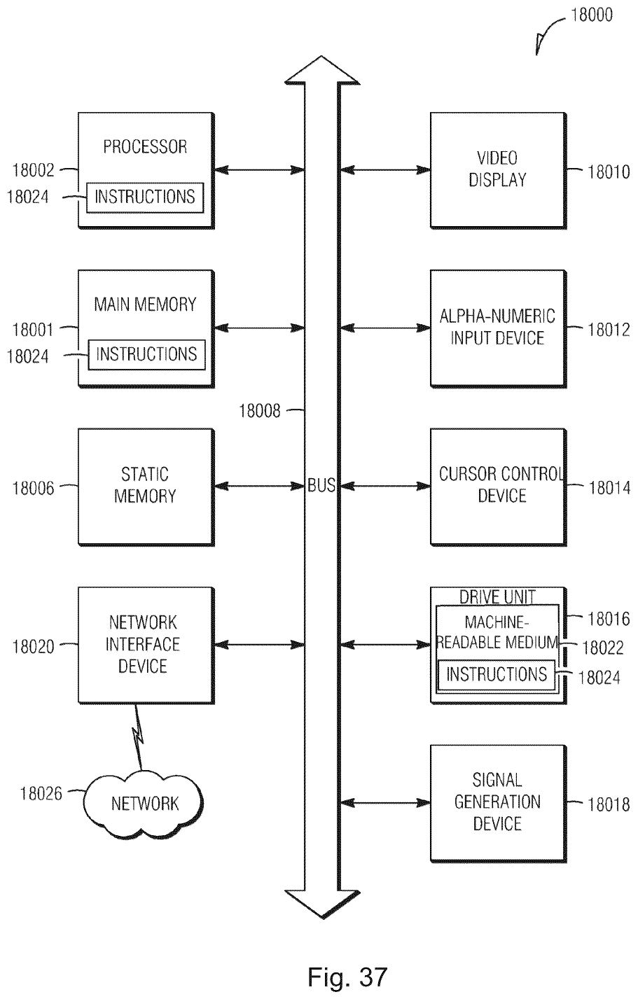

FIG. 37 shows a diagrammatic representation of a machine in the example form of a computer system 18000 within which a set of instructions for causing the machine to perform any one or more of the methods, processes, operations, or methodologies discussed herein may be executed. In alternative embodiments, the machine operates as a standalone device or may be connected (e.g., networked) to other machines. In a networked deployment, the machine may operate in the capacity of a server or a client machine in server-client network environment, or as a peer machine in a peer-to-peer (or distributed) network environment. The machine may be a Personal Computer (PC), a tablet PC, a Personal Digital Assistant (PDA), a cellular telephone or smartphone, a Web appliance, or any machine capable of executing a set of instructions (sequential or otherwise) that specify actions to be taken by that machine. Further, while only a single machine is illustrated, the term “machine” shall also be taken to include any collection of machines that individually or jointly execute a set (or multiple sets) of instructions to perform any one or more of the methodologies discussed herein. Example embodiments may also be practiced in distributed system environments where local and remote computer systems which that are linked (e.g., either by hardwired, wireless, or a combination of hardwired and wireless connections) through a network, both perform tasks. In a distributed system environment, program modules may be located in both local and remote memory-storage devices (see below).

The example computer system 18000 includes a processor 18002 (e.g., a Central Processing Unit (CPU), a Graphics Processing Unit (GPU) or both), a main memory 18001 and a static memory 18006, which communicate with each other via a bus 18008. The computer system 18000 may further include a video display unit 18010 (e.g., a Liquid Crystal Display (LCD) or a Cathode Ray Tube (CRT)). The computer system 18000 also includes an alphanumeric input device 18012 (e.g., a keyboard), a User Interface (UI) cursor controller 18014 (e.g., a mouse), a disk drive unit 18016, a signal generation device 18018 (e.g., a speaker) and a network interface device 18020 (e.g., a transmitter).

The disk drive unit 18016 includes a machine-readable medium 18022 on which is stored one or more sets of instructions 18024 and data structures (e.g., software) embodying or used by one or more of the methodologies or functions illustrated herein. The software may also reside, completely or at least partially, within the main memory 18001 and/or within the processor 18002 during execution thereof by the computer system 18000, the main memory 18001 and the processor 18002 also constituting machine-readable media.

The instructions 18024 may further be transmitted or received over a network 18026 via the network interface device 18020 using any one of a number of well-known transfer protocols (e.g., HTTP, Session Initiation Protocol (SIP)).

The term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding, or carrying a set of instructions for execution by the machine and that cause the machine to perform any of the one or more of the methodologies illustrated herein. The term “machine-readable medium” shall accordingly be taken to include, but not be limited to, solid-state memories, and optical and magnetic medium.

Method embodiments illustrated herein may be computer-implemented. Some embodiments may include computer-readable media encoded with a computer program (e.g., software), which includes instructions operable to cause an electronic device to perform methods of various embodiments. A software implementation (or computer-implemented method) may include microcode, assembly language code, or a higher-level language code, which further may include computer readable instructions for performing various methods. The code may form portions of computer program products. Further, the code may be tangibly stored on one or more volatile or non-volatile computer-readable media during execution or at other times. These computer-readable media may include, but are not limited to, hard disks, removable magnetic disks, removable optical disks (e.g., compact disks and digital video disks), magnetic cassettes, memory cards or sticks, Random Access Memories (RAMs), Read Only Memories (ROMs), and the like.

The specification and drawings are, accordingly, to be regarded in an illustrative rather than a restrictive sense. It will, however, be evident that various modifications and changes may be made thereunto without departing from the broader spirit and scope of the present application as set forth in the claims.

Other variations are within the spirit of the present disclosure. Thus, while the disclosed techniques are susceptible to various modifications and alternative constructions, certain illustrated embodiments thereof are shown in the drawings and have been described above in detail. It should be understood, however, that there is no intention to limit the present application to the specific form or forms disclosed, but on the contrary, the intention is to cover all modifications, alternative constructions and equivalents falling within the spirit and scope of the present application, as defined in the appended claims.

The use of the terms “a” and “an” and “the” and similar referents in the context of describing the disclosed embodiments (especially in the context of the following claims) are to be construed to cover both the singular and the plural, unless otherwise indicated herein or clearly contradicted by context. The terms “comprising,” “having,” “including” and “containing” are to be construed as open ended terms (i.e., meaning “including, but not limited to,”) unless otherwise noted. The term “connected,” when unmodified and referring to physical connections, is to be construed as partly or wholly contained within, attached to or joined together, even if there is something intervening.

Recitation of ranges of values herein are merely intended to serve as a shorthand method of referring individually to each separate value falling within the range, unless otherwise indicated herein and each separate value is incorporated into the specification as if it were individually recited herein. The use of the term “set” (e.g., “a set of items”) or “subset” unless otherwise noted or contradicted by context, is to be construed as a nonempty collection comprising one or more members.

Further, unless otherwise noted or contradicted by context, the term “subset” of a corresponding set does not necessarily denote a proper subset of the corresponding set, but the subset and the corresponding set may be equal.

Operations of processes described herein can be performed in any suitable order unless otherwise indicated herein or otherwise clearly contradicted by context. Processes described herein (or variations and/or combinations thereof) may be performed under the control of one or more computer systems configured with executable instructions and may be implemented as code (e.g., executable instructions, one or more computer programs or one or more applications) executing collectively on one or more processors, by hardware or combinations thereof. The code may be stored on a computer-readable storage medium, for example, in the form of a computer program comprising a plurality of instructions executable by one or more processors. The computer-readable storage medium may be non-transitory.

Preferred embodiments of this disclosure are described herein, including the best mode known to the inventors for carrying out the present application. Variations of those preferred embodiments may become apparent to those of ordinary skill in the art upon reading the foregoing description. The inventors expect skilled artisans to employ such variations as appropriate and the inventors intend for embodiments of the present disclosure to be practiced otherwise than as specifically described herein. Accordingly, the scope of the present disclosure includes all modifications and equivalents of the subject matter recited in the claims appended hereto as permitted by applicable law. Moreover, any combination of the above-described elements in all possible variations thereof is encompassed by the scope of the present disclosure unless otherwise indicated herein or otherwise clearly contradicted by context.

Additional references are listed below:
Wong D T L, Ko B S, Cameron J D, Nerlekar N, Leung M C H, Malaiapan Y, et al. Transluminal attenuation gradient in coronary computed tomography angiography is a novel noninvasive approach to the identification of functionally significant coronary artery stenosis: a comparison with fractional flow reserve. J Am Coll Cardiol. (2013) 61:1271-9. doi: 10.1016/j.jacc.2012.12.029
Ko B S, Wong D T L, Nørgaard B L, Leong D P, Cameron J D, Gaur S, et al. Diagnostic performance of transluminal attenuation gradient and noninvasive fractional flow reserve derived from 320-detector Row CT angiography to diagnose hemodynamically significant coronary stenosis: an NXT substudy. Radiology. (2016) 279:75-83. doi: 10.1148/radiol.2015150383
Diaz-Zamudio M, Dey D, Schuhbaeck A, Nakazato R, Gransar H, Slomka P J, et al. Automated quantitative plaque burden from coronary CT angiography noninvasively predicts hemodynamic significance by using fractional flow reserve in intermediate coronary lesions. Radiology. (2015) 276:408-15. doi:
Otaki Y, Han D, Klein E, Gransar H, Park R H, Tamarappoo B, et al. Value of semiquantitative assessment of high-risk plaque features on coronary CT angiography over stenosis in selection of studies for FFRct. J Cardiovasc Comput Tomogr. (2021) 16:27-33. doi: 10.1016/j.jcct.2021.06.004
Gould K L, Lipscomb K, Calvert C. Compensatory changes of the distal coronary vascular bed during progressive coronary constriction. Circulation. (1975) 51:1085-94. doi: 10.1161/01.CIR.51.6.1085
Dey D, Achenbach S, Schuhbaeck A, Pflederer T, Nakazato R, Slomka P J, et al. Comparison of quantitative atherosclerotic plaque burden from coronary CT angiography in patients with first acute coronary syndrome and stable coronary artery disease. J Cardiovasc Comput Tomogr. (2014) 8:368-74. doi:
Hell M M, Dey D, Marwan M, Achenbach S, Schmid J, Schuhbaeck A. Noninvasive prediction of hemodynamically significant coronary artery stenoses by contrast density difference in coronary CT angiography. Eur J Radiol. (2015) 84:1502-8. doi: 10.1016/j.ejrad.2015.04.024
Ko B S, Wong D T L, Cameron J D, Leong D P, Soh S, Nerlekar N, et al. The ASLA score: a C T angiographic index to predict functionally significant coronary stenoses in lesions with intermediate severity-diagnostic accuracy. Radiology. (2015) 276:91-101. doi: 10.1148/radiol.15141231
Dey D, Gaur S, Ovrehus K A, Slomka P J, Betancur J, Goeller M, et al. Integrated prediction of lesion-specific ischaemia from quantitative coronary CT angiography using machine learning: a multicentre study. Eur Radiol. (2018) 28:2655-64. doi: 10.1007/s00330-017-5223-z
Yang S, Koo B K, Hoshino M, Lee J M, Murai T, Park J, et al. C T angiographic and plaque predictors of functionally significant coronary disease and outcome using machine learning. JACC Cardiovascular imaging. (2021) 14:629-41. doi: 10.1016/j.jcmg.2020.08.025
Ghanem A M, Hamimi A H, Matta J R, Carass A, Elgarf R M, Gharib A M, et al. Automatic coronary wall and atherosclerotic plaque segmentation from 3D coronary CT angiography. Sci Rep. (2019) 9:47. doi: 10.1038/s41598-018-37168-4
Loshchilov, I., Hatter, F., 2019. Decoupled Weight Decay Regularization, in: International Conference on Learning Representations—ICLR 2019

All references, including publications, patent applications and patents, cited herein are hereby incorporated by reference to the same extent as if each reference were individually and specifically indicated to be incorporated by reference and were set forth in its entirety herein.

### Enumerated Clauses:

Enumerated clauses are now provided for the purpose of illustrative some possible embodiments that may be provided in accordance with the disclosure. The clause sets provided below are for illustration and not to be construed as limiting, exclusive or exhaustive. Features recited in one clause set may be utilized and incorporated into one or more of the other clause sets. In any one or more of the following clause sets, embodiments may provide a computer implemented method.

### Clause Set A1:

Embodiments disclosed herein may provide methods and systems for assessing obstruction of a vessel of interest of a patient, which involve one, some or all of the following operations:
obtaining a volumetric image dataset for the vessel of interest;
analyzing the volumetric image dataset to extract data representing axial trajectory of the vessel of interest;
generating a multi-planar reformatted (MPR) image based on the volumetric image dataset and the data representing axial trajectory of the vessel of interest;
generating first feature data that characterizes presence of zero or more bifurcations or side branches along the axial trajectory of the vessel of interest;
supplying the MPR image and first feature data to a first machine learning network that outputs i) a plurality of latent space encodings that characterizes features of the vessel of interest along the axial trajectory of the vessel of interest given the MPR image and the first feature data and ii) additional feature data that characterizes additional features of the vessel of interest along the axial trajectory of the vessel of interest; and
supplying the plurality of latent space encodings and the additional feature data output by the first machine learning network to a second machine learning network that outputs data that characterizes FFR pullback of the vessel of interest given the input data.

A2. The method according to clause A1, which further involves displaying or outputting the data that characterizes FFR pullback of the vessel of interest.

A3. A method according to clause A1 or A2, wherein:
the first feature data is generated from analysis of the MPR image; and/or the first feature data is generated from analysis of the volumetric image dataset;
and/or the first feature data is generated from a coronary artery centerline tree derived from the volumetric image dataset.

A4. A method according to clause A1, A2 or A3, wherein:
the additional features characterized by the additional feature data output by the first machine learning network includes at least one feature related to lumen characteristics of the vessel of interest (such as lumen area and/or lumen attenuation) along the axial trajectory of the vessel of interest.

A5. A method according to clause A1, A2 or A3, wherein:
the additional features characterized by the additional feature data output by the first machine learning network includes at least one feature related to plaque characteristics of the vessel of interest (such as calcium plaque area, soft plaque area, mixed plaque area) along the axial trajectory of the vessel of interest.

A6. A method according to any one of clauses A1 to A5, which further involves generating myocardium feature data that characterizes a localized part of the myocardium that is associated with the vessel of interest, and supplying the myocardium feature data as input to the second machine leaning network for use in generating the data that characterizes FFR pullback of the vessel of interest.

A7. A method according to any one of clauses A1 to A6, wherein:
the first machine learning network comprises a variational autoencoder having an encoder part that generates the plurality of latent space encodings, wherein the encoder part is trained using unsupervised learning.

A8. A method according to clause A7, wherein:
the first feature data is input to convolution blocks of the encoder part.

A9. A method according to clause A7, wherein:
the first machine learning network includes at least one auxiliary decoder part that is supplied with a subset of the latent space encodings generated by the encoder part and configured to generate the additional feature data given the subset of the latent space encodings as input.

A10. A method according to clause A7, wherein:
the at least one auxiliary decoder party is trained by supervised learning using training data that includes reference annotations based on measurements or extraction of corresponding features for a plurality of patients.

A11. A method according to any one of clauses A1 to A10, wherein:
the second machine learning network is trained by supervised learning using training data that includes reference annotations based on measurements of FFR pullback or FFR drop values associated with vessel centerline points for a plurality of patients.

A12. A method according to any one of clauses A1 to A11, wherein:
the second machine learning network is further configured to output an FFR value for a vessel; and
the second machine is trained by supervised learning using training data that includes reference annotations based on measurements of FFR values associated with vessels for a plurality of patients.

A13. A method according to any one of clauses A1 to A12, wherein:
the second machine learning network is further configured to output data that represents a prediction for the presence of a functionally significant stenosis; and
the second machine learning network is trained by supervised learning using training data that includes reference annotations representing presence of a functionally significant stenosis for a plurality of patients.

A14. A method according to any one of clauses A1 to A13, wherein:
the second machine learning network comprises a convolutional neural network, which is trained by supervisory learning using training data that includes reference annotations for the output data of the second machine learning network.

A15. A method according to clause A14, wherein:
the reference annotations are derived by manual segmentation of the corresponding volumetric image data and/or automatic segmentation of the corresponding volumetric image data.

A16. A method according to clause A14, wherein:
the convolutional neural network of the second machine learning system includes a regression head that generates FFR drop along the axial trajectory of the vessel of interest and an output stage that generates the FFR pullback output by the second machine learning system.

A17. A method according to clause A14, wherein:
the convolutional neural network of the second machine learning system further includes a first classification head that outputs data representing an FFR value for a vessel.

A18. A method according to clause A14, wherein:
the convolutional neural network of the second machine learning system further includes a second classification head that outputs data representing a prediction for the presence of a functionally significant stenosis.

A19. A method according to any one of clauses A1 to A18, wherein:
the vessel of interest comprises a coronary artery or a coronary tree.

A20. A method according to any one of clauses A1 to A19, wherein:
the volumetric image dataset comprises CCTA image data.

A21. A system for assessing obstruction of a vessel of interest of a patient, the system comprising:
at least one processor that, when executing program instructions stored in memory, is configured to perform some or all of the operations of clauses A1 to A20.

A22. A system according to clause A21, further comprising:
an imaging acquisition subsystem configured to acquire the volumetric image dataset.

A23. A system according to clause A22, further comprising:
a display subsystem configured to display the data that characterizes anatomical lesion severity of the vessel of interest.

A24. A non-transitory program storage device tangibly embodying a program of instructions that are executable on a machine to perform any or some of the operations of clauses A1 to A20 for assessing obstruction of a vessel of interest of a patient.

### Clause Set B1:

Embodiments disclosed herein may provide methods and systems involving simulating or planning interventional treatment of obstruction of a vessel of interest of a patient, which includes one, some or all of the following operations:
obtaining a volumetric image dataset for the vessel of interest;
analyzing the volumetric image dataset to extract data representing axial trajectory of the vessel of interest;
generating a multi-planar reformatted (MPR) image based on the volumetric image dataset and the data representing axial trajectory of the vessel of interest;
supplying the MPR image to a first machine learning network that outputs i) a plurality of latent space encodings that characterizes features of the vessel of interest along the axial trajectory of the vessel of interest given the MPR image and ii) additional feature data that characterizes additional features of the vessel of interest along the axial trajectory of the vessel of interest; and
adjusting some or all of the additional feature data based on simulated or planned treatment of the vessel of interest;
supplying the plurality of latent space encodings and the adjusted additional feature data output by the first machine learning network to a second machine learning network that outputs data that characterizes FFR pullback of the vessel of interest which accounts for the simulated or planned treatment of the vessel of interest given the input data.

The operations of clause B1 can follow any or some of the operations of clauses A1 to A20 above.

### Clause Set B2:

Embodiments disclosed herein may provide methods and systems involving simulating or planning interventional treatment of obstruction of a vessel of interest of a patient, which include one, some or all of the following operations:
obtaining a volumetric image dataset for the vessel of interest;
analyzing the volumetric image dataset to extract data representing axial trajectory of the vessel of interest;
generating a multi-planar reformatted (MPR) image based on the volumetric image dataset and the data representing axial trajectory of the vessel of interest;
adjusting the MRP image based on simulated or planned treatment of the vessel of interest;
supplying the adjusted MPRG image to a first machine learning network that outputs i) a plurality of latent space encodings that characterizes features of the vessel of interest along the axial trajectory of the vessel of interest given the MPR image and ii) additional feature data that characterizes additional features of the vessel of interest along the axial trajectory of the vessel of interest; and
supplying the plurality of latent space encodings and the additional feature data output by the first machine learning network to a second machine learning network that outputs data that characterizes FFR pullback of the vessel of interest which accounts for the simulated or planned treatment of the vessel of interest given the input data.

The operations of clause B2 can follow any or some of the operations of clauses A1 to A20 and/or B1 above.

B3. The method according to clause B1 or B2, which further involves displaying or outputting the data that characterizes FFR pullback of the vessel of interest which accounts for the simulated or planned treatment of the vessel of interest.

B4. The method according to clause B1 or B2 or B3, which further involves generating first feature data that characterizes presence of zero or more bifurcations or side branches along the axial trajectory of the vessel of interest and supplying the first feature data for input to the first machine learning system for use in generating the plurality of latent space encodings and the additional feature data.

B5. A method according to clause B4, wherein:
the first feature data is generated from analysis of the MPR image; and/or
the first feature data is generated from analysis of the volumetric image dataset; and/or
the first feature data is generated from a coronary artery centerline tree derived from the volumetric image dataset.

B6. A method according to any one of clauses B1 to B5, wherein:
the additional features characterized by the additional feature data output by the first machine learning network (and possible adjusted by the method in B1) includes at least one feature related to lumen characteristics of the vessel of interest (such as lumen area and/or lumen attenuation) along the axial trajectory of the vessel of interest.

B7. A method according to any one of clauses B1 to B6, wherein:
the additional features characterized by the additional feature data output by the first machine learning network (and possibly adjusted by the method in B1) includes at least one feature related to plaque characteristics of the vessel of interest (such as calcium plaque area, soft plaque area, mixed plaque area) along the axial trajectory of the vessel of interest.

B8. A method according to any one of clauses B1 to B7, which further involves generating myocardium feature data that characterizes a localized part of the myocardium that is associated with the vessel of interest, and supplying the myocardium feature data as input to the second machine leaning network for use in generating the data that characterizes FFR pullback of the vessel of interest.

B9. A method according to any one of clauses B1 to B8, wherein:
the first machine learning network comprises a variational autoencoder having an encoder part that generates the plurality of latent space encodings, wherein the encoder part is trained using unsupervised learning.

B10. A method according to clause B1 to B9, wherein:
the first feature data of clause B3 is input to convolution blocks of the encoder part.

B11. A method according to clause B1 to B9, wherein:
the first machine learning network includes at least one auxiliary decoder part that is supplied with a subset of the latent space encodings generated by the encoder part and configured to generate the additional feature data given the subset of the latent space encodings as input.

B12. A method according to clause B1 to B9, wherein:
the at least one auxiliary decoder party is trained by supervised learning using training data that includes reference annotations based on measurements or extraction of corresponding features for a plurality of patients.

B13. A method according to any one of clauses B1 to B12, wherein:
the second machine learning network is trained by supervised learning using training data that includes reference annotations based on measurements of FFR pullback or FFR drop values associated with vessel centerline points for a plurality of patients.

B14. A method according to any one of clauses B1 to B13, wherein:
the second machine learning network is further configured to output an FFR value for a vessel; and
the second machine is trained by supervised learning using training data that includes reference annotations based on measurements of FFR values associated with vessels for a plurality of patients.

B15. A method according to any one of clauses B1 to B14, wherein:
the second machine learning network is further configured to output data that represents a prediction for the presence of a functionally significant stenosis; and
the second machine learning network is trained by supervised learning using training data that includes reference annotations representing presence of a functionally significant stenosis for a plurality of patients.

B16. A method according to any one of clauses B1 to B15, wherein:
the second machine learning network comprises a convolutional neural network, which is trained by supervisory learning using training data that includes reference annotations for the output data of the second machine learning network.

B17. A method according to clause B16, wherein:
the reference annotations are derived by manual segmentation of the corresponding volumetric image data and/or automatic segmentation of the corresponding volumetric image data.

B18. A method according to clause B16 or B17, wherein:
the convolutional neural network of the second machine learning system includes a regression head that generates FFR drop along the axial trajectory of the vessel of interest and an output stage that generates the FFR pullback output by the second machine learning system.

B19. A method according to clause B16 to B18, wherein:
the convolutional neural network of the second machine learning system further includes a first classification head that outputs data representing an FFR value for a vessel.

B20. A method according to clause B16 to B19, wherein:
the convolutional neural network of the second machine learning system further includes a second classification head that outputs data representing a prediction for the presence of a functionally significant stenosis.

B21. A method according to any one of clauses B1 to B20, wherein:
the vessel of interest comprises a coronary artery or a coronary tree.

B22. A method according to any one of clauses B1 to B21, wherein:
the volumetric image dataset comprises CCTA image data.

B23. A system for assessing obstruction of a vessel of interest of a patient, the system comprising:
at least one processor that, when executing program instructions stored in memory, is configured to perform any or some of the operations of clauses B1 to B22.

B24. A system according to clause B23, further comprising:
an imaging acquisition subsystem configured to acquire the volumetric image dataset.

B25. A system according to clause B23, further comprising:
a display subsystem configured to display the data that characterizes anatomical lesion severity of the vessel of interest.

B26. A non-transitory program storage device tangibly embodying a program of instructions that are executable on a machine to perform any or some of the operations of clauses B1 to B22 involving simulation or planning of treatment of an obstruction of a vessel of interest of a patient.

### Clause Set C1:

Embodiments disclosed herein may provide methods and systems for extracting a coronary tree from volumetric image data of a vessel of interest of a patient, which include one, some or all of the following operations:
obtaining a volumetric image dataset for the vessel of interest;
tracking a plurality of seed points in the image dataset;
using the plurality of seed points to extract an initial representation of a coronary tree in the image dataset;
inputting the initial representation of the coronary tree to a first ensemble of graph
convolutional neural networks to generate a refined representation of the coronary tree; and
using a second ensemble of graph convolutional neural networks to generate labels for segments of the refined representation of the coronary tree.

C2. A method according to clause C1, wherein:
the initial representation and refined representation of the coronary tree represents the coronary tree as an undirected tree graph, wherein each point in the centerline of coronary segments corresponds to a node in the tree graph and the connections between centerline points are represented by undirected edges in the tree graph.

C3. A method according to clause C1 or C2, wherein:
the initial representation of the coronary tree is built by tracking coronary centerlines from the seed points.

C4. A method according to clause C1 or C2 or C3, wherein:
the initial representation of the coronary tree is derived by predicting seed points and the location of the coronary ostia using two convolutional neural networks.

C5. A method according to any one of clauses C1 to C4, wherein:
the initial representation of the coronary tree is derived by add new nodes to the tree using a convolutional neural network configured to predict a direction and a step size from one or more end nodes (node with fewer than two edges) of the graph to generate resultant sub-graphs, and merging the sub-graphs when overlapping is present.

C6. A method according to any one of clauses C1 to C5, wherein:
the initial representation of the coronary tree is derived by creating segments by grouping adjacent centerline points of the tree graph.

C7. A method according to clause C6, wherein:
the segments are characterized using a set of features selected from the group including: location, orientation, geometry, or image appearance of the segments.

C8. A method according to any one of clauses C1 to C7, wherein:
the first ensemble of graph convolutional neural networks is configured to perform binary classification that distinguish real coronary artery segments (positive class) from other vessel-like structures (negative class),

C9. A method according to any one of clauses C1 to C8, wherein:
the first ensemble of graph convolutional neural networks is configured to employ a multiresolution graph ensembling strategy where predictions for multiple graphs with different resolutions are back projected to a fine graph.

C10. A method according to any one of clauses C1 to C9, wherein:
the second ensemble of graph convolutional neural networks is trained on graphs with different resolutions for anatomical labeling.

C11. The method according to any one of clauses C1 to C10, which further involves displaying or outputting the refined representation of the coronary tree and/or the labels for the segments of the coronary tree.

C12. A system for extracting a coronary tree from volumetric image data of a vessel of interest of a patient, the system comprising:
at least one processor that, when executing program instructions stored in memory, is configured to perform any or some of the operations of clauses C1 to C12.

C13. A system according to clause C3, further comprising:
an imaging acquisition subsystem configured to acquire the volumetric image dataset.

C14. A system according to clause C13, further comprising:
a display subsystem configured to display to any one of clauses C1 to C10, which further involves displaying or outputting the refined representation of the coronary tree and/or the labels for the segments of the coronary tree.

C15. A non-transitory program storage device tangibly embodying a program of instructions that are executable on a machine to perform any or some of the operations of clauses C1 to C11 to extract coronary tree from volumetric image data.

All the features appearing in the clause sets above can be combined among them and with any feature appearing in the appended claims.

1. A method for assessing obstruction of a vessel of interest of a patient, comprising:
obtaining a volumetric image dataset for the vessel of interest;
analyzing the volumetric image dataset to extract data representing axial trajectory of the vessel of interest;
generating a multi-planar reformatted (MPR) image based on the volumetric image dataset and the data representing axial trajectory of the vessel of interest;
supplying the MPR image as input to a first machine learning network that outputs feature data that characterizes a plurality of features of the vessel of interest along the axial trajectory of the vessel of interest given the MPR image;
generating additional data that characterizes at least one additional feature of the vessel of interest along the axial trajectory of the vessel of interest by analysis separate and distinct from the first machine learning network; and
supplying the data output by the first machine learning network and the additional data as input data to a second machine learning network that outputs data that characterizes anatomical lesion severity of the vessel of interest given the input data.

2. A method according to claim 1, further comprising:
displaying or outputting the data that characterizes anatomical lesion severity of the vessel of interest.

3. A method according to claim 1, wherein:
the additional data is generated from analysis of the MPR image; and/or
the additional data is generated from analysis of the volumetric image dataset; and/or
the additional data is generated from a coronary artery centerline tree derived from the volumetric image dataset.

4. A method according to claim 1, wherein:
the additional data characterizes at least one of side branches and bifurcations along the axial trajectory of the vessel of interest.

5. A method according to claim 1, wherein:
the additional data characterizes at least one of soft plaque area, mixed plaque area, or other characteristic feature along the axial trajectory of the vessel of interest.

6. A method according to claim 1, wherein:
the additional data further characterizes a localized part of the myocardium that is associated with the vessel of interest.

7. A method according to claim 1, wherein:
the data output by the second machine learning network includes a fractional flow reserve (FFR) value for the entire vessel of interest; and
the second machine learning network is trained by supervised learning using training data that includes reference annotations based on measurements of FFR values for a plurality of patients.

8. A method according to claim 1, wherein:
the data output by the second machine learning network includes fractional flow reserve (FFR) values for centerline points along the vessel of interest; and
the second machine learning network is trained by supervised learning using training data that includes reference annotations based on measurements of FFR values associated with vessel centerline points for a plurality of patients.

9. A method according to claim 1, wherein:
the data output by the second machine learning network represents a prediction for the presence of a functionally significant stenosis; and
the second machine learning network is trained by supervised learning using training data that includes reference annotations representing presence of a functionally significant stenosis for a plurality of patients.

10. A method according to claim 1, wherein:
the plurality of the features characterized by the feature data output by the first machine learning network includes at least one feature related to lumen characteristics of the vessel of interest (such as lumen area and/or lumen attenuation) along the axial trajectory of the vessel of interest.

11. A method according to claim 1, wherein:
the plurality of the features characterized by the feature data output by the first machine learning network includes at least one feature related to plaque characteristics of the vessel of interest (such as calcium plaque area, soft plaque area, mixed plaque area) along the axial trajectory of the vessel of interest.

12. A method according to claim 1, wherein:
the first machine learning network comprises a convolutional neural network, which is trained using training data that includes reference annotations for the plurality of the features characterized by the feature data output by the first machine learning network.

13. A method according to claim 12, wherein:
the reference annotations are derived by manual segmentation of corresponding volumetric image data and/or automatic segmentation of corresponding volumetric image data.

14. A method according to claim 1, wherein:
the second machine learning network comprises a convolutional neural network, which is trained using training data that includes volumetric image data and corresponding reference annotations for the output data that characterizes anatomical lesion severity of the vessel of interest.

15. A method according to claim 14, wherein:
the reference annotations are derived by manual segmentation of the corresponding volumetric image data and/or automatic segmentation of the corresponding volumetric image data.

16. A method according to claim 14, wherein:
the convolutional neural network of the second machine learning system includes a regression head that outputs a fractional flow reserve (FFR) value.

17. A method according to claim 16, wherein:
the convolutional neural network of the second machine learning system further includes an accumulator that outputs fractional flow reserve (FFR) values for centerline points along the vessel of interest.

18. A method according to claim 16, wherein:
the convolutional neural network of the second machine learning system further includes a classification head that outputs data representing a prediction for the presence of a functionally significant stenosis.

19. A method according to claim 1, wherein:
the vessel of interest comprises a coronary artery or a coronary tree.

20. A method according to claim 1, wherein:
the volumetric image dataset comprises CCTA image data.

21. A system for assessing obstruction of a vessel of interest of a patient, the system comprising:
at least one processor that, when executing program instructions stored in memory, is configured to perform the method of claim 1.

22. A system according to claim 21, further comprising:
an imaging acquisition subsystem configured to acquire the volumetric image dataset.

23. A system according to claim 22, further comprising:
a display subsystem configured to display the data that characterizes anatomical lesion severity of the vessel of interest.

24. A non-transitory program storage device tangibly embodying a program of instructions that are executable on a machine to perform the operations of claim 1 for assessing obstruction of a vessel of interest of a patient.
